(19) United States
(12) Patent Application Publication
(10) Pub. No.: US 2024/0327418 A1
(43) Pub. Date: Oct. 3, 2024

(54) MULTI-TARGET INHIBITOR TARGETING HDAC AND NAD SYNTHESIS AND USE OF MULTI-TARGET INHIBITOR

(71) Applicants: OCEAN UNIVERSITY OF CHINA, Qingdao (CN); QINGDAO HIGENE BIOPHARMACEUTICAL CO., LTD, Qingdao (CN); MARINE BIOMEDICAL RESEARCH INSTITUTE OF QINGDAO CO., LTD, Qingdao (CN)

(72) Inventors: Xiaoyang LI, Qingdao (CN); Zhongren ZHOU, Qingdao (CN); Yuqi JIANG, Qingdao (CN); Kairui YUE, Qingdao (CN); Wenfang XU, Qingdao (CN)

(21) Appl. No.: 18/293,840

(22) PCT Filed: Aug. 1, 2022
(86) PCT No.: PCT/CN2022/109469

§ 371 Date: Jan. 31, 2024

(30) Foreign Application Priority Data
Aug. 4, 2021 (CN) 202110892485.X

(51) Int. Cl. C07D 491/048; A61K 31/343; A61K 31/381; A61K 31/404; A61K 31/4355; A61K 31/4365; A61K 31/437; A61K 31/4406; A61K 31/4439; A61K 31/655; A61P 35/00; A61P 35/02; C07C 247/12; C07D 209/42; C07D 213/40; C07D 213/56; C07D 307/85; C07D 333/70; C07D 401/04; C07D 471/04; C07D 495/04

(52) U.S. Cl. CPC C07D 491/048; A61K 31/343; A61K 31/381; A61K 31/404; A61K 31/4355; A61K 31/4365; A61K 31/437; A61K 31/4406; A61K 31/4439; A61K 31/655; A61P 35/00; A61P 35/02; C07C 247/12; C07D 209/42; C07D 213/40; C07D 213/56; C07D 307/85; C07D 333/70; C07D 401/04; C07D 471/04; C07D 495/04

(57) ABSTRACT

A compound targeting HDAC and NAD synthesis and a pharmaceutically acceptable salt, hydrate, deuterate, isomer or prodrug thereof, as well as preparation and application thereof. Specifically, a compound shown in structural general formula (I) and a pharmaceutically acceptable salt, hydrate, deuterate, isomer or prodrug thereof are provided. The compound of the structural general formula (I) is a multi-target inhibitor, targeting HDAC and NAD targets, and exhibiting significant HDAC inhibitory activity, while representative compounds exhibit certain NAD inhibitory activity.

Ring E-B-L-C (O)—(NH)r-R (General formula I)

### TECHNICAL FIELD

The present disclosure belongs to the field of biomedical technology, in particular relates to a multi-target inhibitor targeting HDAC and NAD synthesis, a pharmaceutically acceptable salt, solvate and prodrug thereof, as well as use thereof.

### BACKGROUND

The focus of traditional drug research is to find molecules with high affinity and high selectivity towards a single target. However, the occurrence and development of tumors depend on multiple receptors or signaling pathways, which means that anti-tumor drugs targeting a single target cannot completely kill tumor cells or develop resistance due to compensatory resistance. To overcome these limitations, design of multi-target anti-tumor drugs has been considered an effective strategy and has attracted widespread attentions in drug development. Ideally, multi-target drugs can simultaneously regulate networks of disease-related targets and generate synergistic effects (Proc Natl Acad Sci USA. 2019, 116, 7129-7136). Compared with combination therapy, multi-target drugs can avoid drug-drug interactions, reduce toxic side effects, and improve patient compliance, etc.

Histone deacetylase (HDAC) is a family of enzymes involved in regulating many cellular processes, including cell proliferation, apoptosis, and cytoskeleton assembly. HDAC affects cellular functions by regulating the acetylation levels of histones and nonhistones. This process involves the mutual balance between histone acetyltransferase (HAT) and HDAC, both of which are involved in post-translational modifications of histones (Cancer Chemoth Pharm. 2001, 48, 20-26). HAT and HDAC have opposite effects on highly conserved lysine residues at the N-terminus of acetylated and deacetylated histones, thereby altering chromatin assembly and transcriptional activity. HDAC is also involved in regulating the acetylation of many nonhistones, such as α-tubulin and tumor suppressor p53 (Pharmacol Res. 2021, 163, 105274; J Invest Dermatol. 2020, 140, 2009-2022). These results, as well as reports of abnormal HDAC activity in many tumor types, indicate that HDAC inhibition represents a feasible anti-cancer strategy.

Given the important role of HDAC in tumor development, HDAC has synergistic anti-tumor effects with various tumor targets (such as tubulins and heat shock protein 90 (Hsp90)), which has led to extensive research on multi-target molecules designed based on the inhibition of HDAC (Eur J Med Chem, 2020, 208, 112831). Nicotinamide adenine dinucleotide (NAD+), as the most important coenzyme and core metabolite in organisms, not only widely participates in energy metabolism redox reactions. Due to the rapid proliferation of tumor cells and their higher production capacity requirements, the biosynthesis of NAD⁺ in tumor tissues is often upregulated as well (Nat Rev Cancer. 2012, 12, 741-752). Nicotinamide phosphoribosyltransferase (NAMPT) is one of the most representative metabolic targets. NAMPT catalyzes the production of nicotinamide mononucleotide (NMN) from nicotinamide (NAM) and regulates the level of NAD which is an essential energy substance in mammalian cells (Nat Rev Endocrinol. 2015,11, 535-546). NAMPT is a rate-limiting enzyme in NAD production pathway and plays a crucial role in cellular physiological activities. Research has shown that targeting NAD synthesis has important anti-tumor effects for the reasons that 1) tumor cells have higher NAD consumption and metabolic rates than normal cells, and they are more susceptible to the influence from NAMPT inhibitors; 2) NAD is an essential coenzyme involved in the synthesis of various essential substances in tumor cells, and NAD can significantly reduce the content of reactive oxygen species (ROS) in the environment to protect tumor cells; and 3) NAMPT plays a crucial role in angiogenesis and induces the production of vascular endothelial growth factors. At present, two NAMPT inhibitors, i.e. FK866 and CHS-828 targeting NAD synthesis have entered clinical research (Cancer Res. 2003, 63, 7436-7442; Cancer Res. 1999, 59, 5751-5757).

Targeting HDAC and NAD synthesis has a synergistic anti-tumor effect. Certain specific genotypes, such as p53 deficient or mutated tumor cells, develop primary resistance to HDAC inhibitors, and when combined with NAD synthesis blocking drugs, it is possible to produce Synthetic Lethality effect on these cells, thereby achieving better anti-tumor effects. Therefore, designing multi-target inhibitors targeting HDAC and NAD synthesis is of great significance for tumor treatment. In addition, the pharmacophore analysis of NAMPT and HDAC inhibitors showed that both of them have similar structural characteristics, providing a basis for designing dual inhibitors.

### SUMMARY

The present application specifically provides an HDAC compound with multi-target inhibitory activity and a pharmaceutically acceptable salt, hydrate, deuterate, isomer, or prodrug thereof, wherein the multi-target HDAC compound has a general formula I,

Ring E-B-L-C(O)—(NH)r-R (General formula I)

wherein, ring E is selected from

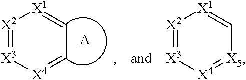

r=1 and 2, and R is equal to 1 and 2, R is selected from H, C₁₋₄ alkyl, C₃₋₅ cycloalkyl or C₁₋₂ alkyl substituted C₃₋₅ cycloalkyl, and

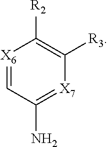

More further, the compound of general formula I is further selected from compounds of general formula II, general formula III, general formula IV, and general formula V:

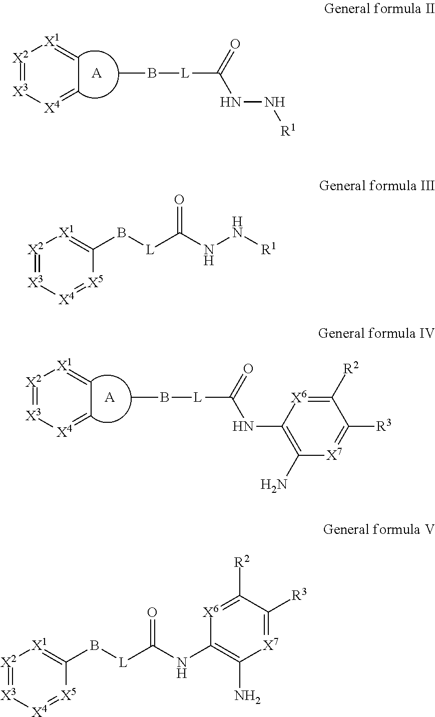

### General Formula V

or a pharmaceutically acceptable salt, hydrate, deuterate, or prodrug thereof, wherein:

ring A is selected from

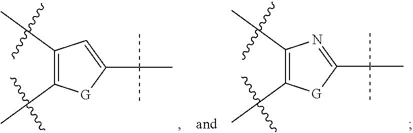

G is selected from CH₂, NH, N(CH₂)ₙCH₃, O or S, wherein n is from 0 to 9;

[symbol] represents a bond connected to an adjacent fused ring;

[symbol] represents a bond connected to B;

X¹ is selected from CR⁴ or N;

X² is selected from CR⁵ or N;

X³ is selected from CR⁶ or N;

X⁴ is selected from CR⁷ or N;

X⁵, X⁶ or X⁷ are independently selected from CH or N;

R¹ is selected from H, C₁-C₄ alkyl, C₃-C₅ cycloalkyl, or C₁-C₂ alkyl substituted C₃-C₅ cycloalkyl;

R² and R³ are each independently selected from H, halogen, CH₃, and OCH₃;

B is selected from

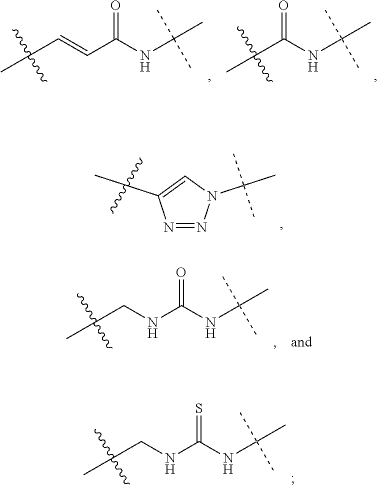

[symbol] represents a bond connected to ring A;

[symbol] represents a bond connected to L;

L is selected from the group consisting of C₁₋₁₄ alkyl, C₁₋₁₄ alkoxy, C₂₋₁₄ alkenyl, C₂₋₁₄ alkynyl, C₃₋₁₀ cycloalkyl, C₁₋₉ alkyl substituted C₃₋₁₀ cycloalkyl, C₁₋₉ alkoxy substituted C₃₋₁₀ cycloalkyl, C₆₋₁₀ aryl, C₁₋₉ alkyl substituted C₆₋₁₀ aryl, C₁₋₉ alkoxy substituted C₆₋₁₀ aryl, (C₁₋₉ alkyl))-(C═O)NH substituted aryl, benzyl, or (C₁₋₈ alkyl)-(C═O)NH substituted benzyl;

R⁴, R⁵, R⁶, and R⁷ are independently selected from H, halogen, (C₁₋₂) alkyl, halomethyl, OH, OCH₃, O(CH₂),CH₃, cyclopropyloxy, OC(CH₃)₃, OCH(CH₃)₂, 5 to 6-membered alkoxy, NH₂, N(CH₃)₂, NH(CH₂)ₙCH₃, CN, N₃, etc., wherein n is from 0 to 9.

Compounds as shown in general formulas I-V and a pharmaceutically acceptable salt, hydrate, deuterate, isomer, or prodrug thereof are preferably defined in the present disclosure,

wherein,

Ring A is selected from the following ring systems:

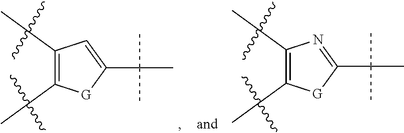

G is selected from NH, O or S;

[symbol] represents a bond connected to an adjacent fused ring;

[symbol] represents a bond connected to B;

X¹ is selected from CR⁴ or N;

X² is selected from CR⁵ or N;

X³ is selected from CR⁶ or N;

X⁴ is selected from CR⁷ or N;

R⁴, R⁵, R⁶ and R⁷ are all H;

R¹ is selected from H, C₁₋₄ alkyl, C₃₋₅ cycloalkyl or C₁₋₂ alkyl substituted C₃₋₅ cycloalkyl;

B is selected from the following structures

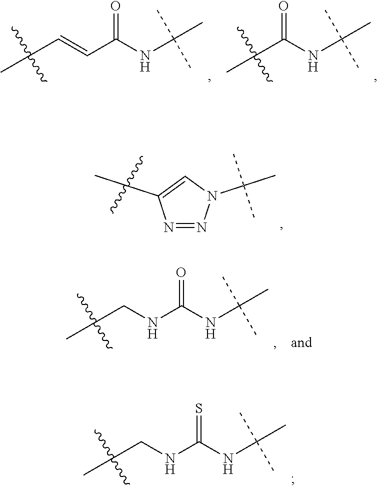

[symbol] represents a bond connected to ring A;

[symbol] represents a bond connected to L;

L is selected from C₁₋₁₄ alkyl, C₁₋₁₄ alkoxy, C₂₋₁₄ alkenyl, C₂₋₁₄ alkynyl, C₁₋₉ alkyl substituted C₃₋₁₀ cycloalkyl, C₁₋₉ alkoxy substituted C₃₋₁₀ cycloalkyl, C₆₋₁₀ aryl, C₁₋₉ alkyl substituted C₆₋₁₀ aryl, C₁₋₉ alkoxy substituted C₆₋₁₀ aryl, (C₁₋₉ alkyl))-(C═O)NH substituted aryl, benzyl, or (C₁₋₈ alkyl)-(C═O)NH substituted benzyl;

Compounds as shown in formula III and a pharmaceutically acceptable salt, hydrate, deuterate, isomer, or prodrug thereof are preferably defined in the present disclosure,

wherein,

X¹ is selected from CR⁴ or N;

X² is selected from CR⁵ or N;

X³ is selected from CR⁶ or N;

X⁴ is selected from CR⁷ or N;

X⁵ is selected from CH or N;

R⁴, R⁵, R⁶ and R⁷ are all H;

R¹ is selected from H, C₁₋₄ alkyl, C₃₋₅ cycloalkyl or C₁₋₂ alkyl substituted C₃₋₅ cycloalkyl;

B is selected from the following structures

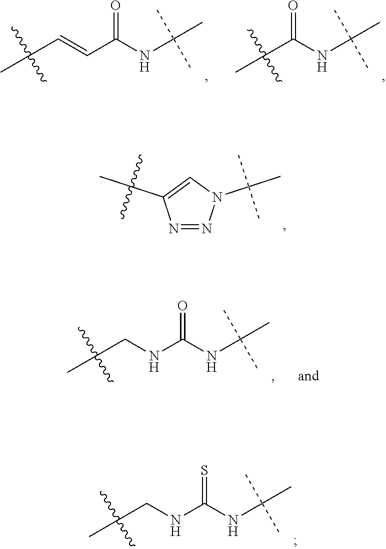

[symbol] represents a bond connected to ring A;

[symbol] represents a bond connected to L;

L is selected from C₁₋₁₄ alkyl, C₁₋₁₄ alkoxy, C₂₋₁₄ alkenyl, C₂₋₁₄ alkynyl, C₁₋₉ alkyl substituted C₃₋₁₀ cycloalkyl, C₁₋₉ alkoxy substituted C₃₋₁₀ cycloalkyl, C₆₋₁₀ aryl, C₁₋₉ alkyl substituted C₆₋₁₀ aryl, C₁₋₉ alkoxy substituted C₆₋₁₀ aryl, (C₁₋₉ alkyl))-(C═O)NH substituted aryl, benzyl, or (C₁₋₈ alkyl)-(C═O)NH substituted benzyl;

Compounds as shown in formula IV and a pharmaceutically acceptable salt, hydrate, deuterate, isomer, or prodrug thereof are preferably defined in the present disclosure,

wherein,

Ring A is selected from the following ring systems:

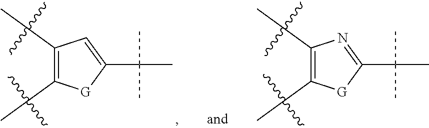

G is selected from NH, O or S;

[symbol] represents a bond connected to an adjacent fused ring;

[symbol] represents a bond connected to B;

X¹ is selected from CR⁴ or N;

X² is selected from CR⁵ or N;

X³ is selected from CR⁶ or N;

X⁴ is selected from CR⁷ or N;

X⁶, or X⁷ is independently selected from CH or N;

R⁴, R⁵, R⁶ and R⁷ are all H;

R¹ is selected from H, C₁₋₄ alkyl, C₃₋₅ cycloalkyl or C₁₋₂ alkyl substituted C₃₋₅ cycloalkyl;

R² and R³ are both H;

B is selected from the following structures

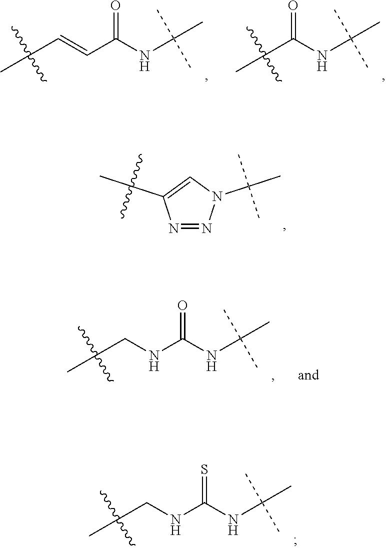

[symbol] represents a bond connected to ring A;

[symbol] represents a bond connected to L;

L is selected from C₁₋₁₄ alkyl, C₁₋₁₄ alkoxy, C₂₋₁₄ alkenyl, C₂₋₁₄ alkynyl, C₁₋₉ alkyl substituted C₃₋₁₀ cycloalkyl, C₁₋₉ alkoxy substituted C₃₋₁₀ cycloalkyl, C₆₋₁₀ aryl, C₁₋₉ alkyl substituted C₆₋₁₀ aryl, C₁₋₉ alkoxy substituted C₆₋₁₀ aryl, (C₁₋₉ alkyl)-(C═O)NH substituted aryl, benzyl, or (C₁₋₈ alkyl)-(C═O)NH substituted benzyl;

Compounds as shown in formula V and a pharmaceutically acceptable salt, hydrate, deuterate, isomer, or prodrug thereof are preferably defined in the present disclosure,

wherein,

X¹ is selected from CR⁴ or N;

X² is selected from CR⁵ or N;

X³ is selected from CR⁶ or N;

X⁴ is selected from CR⁷ or N;

X⁵, X⁶ or X⁷ are independently selected from CH or N

R⁴, R⁵, R⁶ and R⁷ are all H;

R¹ is selected from H, C₁₋₄ alkyl, C₃₋₅ cycloalkyl or C₁₋₂ alkyl substituted C₃₋₅ cycloalkyl;

B is selected from the following structures

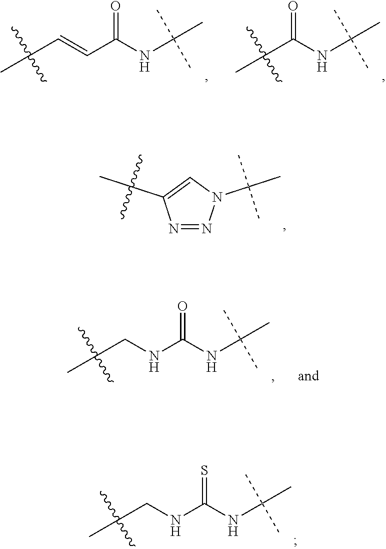

[symbol] represents a bond connected to ring A;

[symbol] represents a bond connected to L;

L is selected from C₁₋₁₄ alkyl, C₁₋₁₄ alkoxy, C₂₋₁₄ alkenyl, C₂₋₁₄ alkynyl, C₁₋₉ alkyl substituted C₃₋₁₀ cycloalkyl, C₁₋₉ alkoxy substituted C₃₋₁₀ cycloalkyl, C₆₋₁₀ aryl, C₁₋₉ alkyl substituted C₆₋₁₀ aryl, C₁₋₉alkoxy substituted C₆₋₁₀ aryl, (C₁₋₉ alkyl)-(C═O)NH substituted aryl, benzyl, or (C₁₋₈ alkyl)-(C═O)NH substituted benzyl;

Unless specifically defined, the compounds and salts provided herein may also include all isotopes of atoms present in intermediates or final compounds. The isotopes include those atoms with the same atomic number but different mass numbers.

Those skilled in the art will understand that the method described is not an exclusive means of synthesizing the compounds provided herein, and can obtain a broad set of synthetic organic reactions, thereby potentially being used for synthesizing the compounds provided herein. Those skilled in the art know how to choose and carry out appropriate synthesis routes. The appropriate synthesis methods for starting materials, intermediates, and products can be determined by referring to literature including, for example, the following references: Advances in Heterocyclic Chemistry, Volumes 1-107 (Elsevier, 1963-2012); Journal of Heterocyclic Chemistry, Volumes 1-49 (Journal of Heterocyclic Chemistry, 1964-2012); Carreira et al. (eds.), Science of Synthesis, Volumes 1-48 (2001-2010); Katritzky et al. (eds.), Comprehensive Organic Transformations (Pergamon Press, 1996); Katritzky et al. (eds.); Comprehensive Organic Transformations II (Elsevier, 2nd edition, 2004); Katritzky et al. (eds.), Comprehensive Heterocyclic Chemistry (Pergamon Press, 1984); Smith et al., Advanced Organic Chemistry: Reactions, Mechanisms, and Structures, 6th edition (Wiley, 2007); Trost et al. (eds.), Comprehensive Organic Synthesis (Pergamon Press, 1991).

The preparation of the compounds described herein can involve the protection and deprotection of various chemical groups. The need for protection and deprotection, as well as the selection of appropriate protective groups, can be easily determined by those skilled in the art. It can be found for the chemistry of protective groups in, for example, T W. Greene and P G. M. Wuts, Protective Groups in Organic Synthesis, 3rd Edition, Wiley & Sons, Inc., New York (1999).

The reaction can be monitored using any appropriate methods known in the art. For example, the formation of products can be monitored through the following methods: spectroscopy methods such as nuclear magnetic resonance spectroscopy (e.g., ¹H or ¹³C), infrared spectroscopy, spectrophotometry (e.g., UV-visible), mass spectrometry, or chromatography methods such as high performance liquid chromatography (HPLC), liquid chromatography-mass spectrometry (LCMS), or thin-layer chromatography (TLC). Compounds can be purified by a variety of methods including high performance liquid chromatography (HPLC) and normal phase silica gel chromatography by those skilled in the art.

As used herein, the phrase “optionally substituted” means unsubstituted or substituted. As used herein, the term “substituted” means that a hydrogen atom is removed and replaced with a substituent. It should be understood that substitution on a given atom is limited by its valence.

Throughout all definitions, the term “Cn-m” refers to a range that includes endpoints, where n and m are integers and represent the carbon number. Examples include C₁₋₁₄ and C₂₋₁₄, etc.

As used herein, the term “Cₙ₋ₘ alkyl” used alone or in combination with other terms refers to saturated hydrocarbyl that can be linear or branched and has n to m carbons. Examples of alkyl moiety include, but are not limited to the following chemical groups: methyl, ethyl, n-propyl, isopropyl, n-butyl, tert-butyl, isobutyl, and sec-butyl; higher homologues, such as 2-methyl-1-butyl, n-pentyl, 3-pentyl, n-hexyl, and 1,2,2-trimethylpropyl. In some embodiments, alkyl includes 1 to 14 carbon atoms, 1 to 13 carbon atoms, 1 to 12 carbon atoms, 1 to 11 carbon atoms, 1 to 10 carbon atoms, 1 to 9 carbon atoms, 1 to 8 carbon atoms, 1 to 7 carbon atoms, 1 to 6 carbon atoms, 1 to 5 carbon atoms, 1 to 4 carbon atoms, 1 to 3 carbon atoms, and 1 to 2 carbon atoms.

As used herein, the term “Cₙ₋ₘ alkoxy” used alone or in combination with other terms refers to a group of formula O-alkyl, wherein alkyl has n to m carbons. Examples of alkoxy include methoxy, ethoxyl, propanoxy (e.g., n-propanoxy and isopropoxy), and tert-butoxy, etc. In some embodiments, alkyl has 1 to 6, 1 to 4, or 1 to 3 carbon atoms.

As used herein, “halogen” refers to F, Cl, Br or I. In some embodiments, halogen is F, Cl, or Br. In some embodiments, halogen is F. In some embodiments, halogen is Cl. In some embodiments, halogen is Br. In some embodiments, halogen is I.

As used herein, the term “Cn-m haloalkyl” refers to alkyl having 1 to 2s+1 halogen atoms that can be identical or different, where “s” is the number of carbon atoms in alkyl, and alkyl has n to m carbon atoms. In some embodiments, haloalkyl is only fluorinated (e.g., C₁₋₆ fluoroalkyl). In some embodiments, alkyl has 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2 carbon atoms.

As used herein, the term “aryl” refers to aromatic hydrocarbyl, which can be monocyclic or polycyclic (e.g., having two fused rings). The term “Cn-m aryl” refers to aryl having n to m cyclic carbon atoms. Aryl includes, for example, phenyl, and naphthyl, etc. In some embodiments, aryl has 6 to 10 carbon atoms. In some embodiments, aryl is substituted or unsubstituted phenyl.

As used herein, “cycloalkyl” refers to non-aromatic cyclic hydrocarbons that include cyclized alkyl and/or alkenyl. Cycloalkyl can include monocyclic or polycyclic (e.g., having 2 fused rings) groups. Cycloalkyl can have 3, 4, 5, and 6 ring-forming carbons (i.e., C₃₋₆ cycloalkyl). The ring-forming carbon atoms of cycloalkyl can be optionally substituted by an oxygen group (oxo) or a sulfur group (sulfido) (e.g., C(═O) or C(═S)). Examples of cycloalkyl include but are not limited to cyclopropyl, cyclobutyl, cyclopentyl, cyclohexyl, cycloheptyl, cyclopentenyl, cyclohexenyl or cyclohexadienyl, etc. In some embodiments, cycloalkyl is selected from the group consisting of cyclopropyl, cyclobutyl, cyclopentyl, and cyclohexyl. In some embodiments, cycloalkyl has 3 to 6 ring-forming carbon atoms (i.e., C₃₋₆ cycloalkyl).

As used herein, “heterocycloalkyl” refers to a non-aromatic monocyclic or polycyclic heterocycle having one or more ring-forming heteroatoms selected from O, N, or S. Included in heterocycloalkyl are monocyclic 4-, 5-, and 6-membered heterocycloalkyl. Examples of heterocycloalkyl include pyrrolidin-2-one, 1,3-isoxazolidin-2-one, pyranyl, tetrahydropyranyl, oxetanyl, azetidinyl, morpholino, thiomorpholino, piperazinyl, tetrahydrofuranyl, tetrahydrothienyl, piperidyl, pyrrolidinyl, isooxazolidinyl, isothiazolidinyl, pyrazolidinyl, oxazolidinyl, thiazolidinyl, imidazolidinyl, azaalkanyl, etc. The ring-forming carbon atoms and heteroatoms of heterocycloalkyl can be optionally substituted by oxo (═O). Heterocycloalkyl can be connected by ring-forming carbon atoms or ring-forming heteroatoms. In some embodiments, heterocycloalkyl contains 0 to 3 double bonds.

The term “compound” as used herein means to include all stereoisomers, geometrical isomers, tautomers, and isotopes of the depicted structure. Unless otherwise specified, compounds identified as a specific tautomeric form by name or structure herein are intended to include other tautomeric forms.

The compounds provided herein also include tautomeric forms. The tautomeric form is caused by the exchange between single bonds and adjacent double bonds and the accompanying proton migration. The tautomeric form includes prototropic tautomers, which are in protonated states of isomerisms with the same empirical formula and total charge. Examples of prototropic tautomers include keto-enol pairs, amide-imidic acid pairs, lactam-lactim pairs, enamine-imine pairs, and cyclic forms in which protons can occupy more than two positions in the heterocyclic systems, such as 1H- and 3H-imidazole, 1H-, 2H- and 4H-1,2,4-triazole, 1H- and 2H-isoindole, and 1H- and 2H-pyrazole. The tautomeric form can be in equilibrium or stereologically locked into one form through appropriate substitution.

All compounds and pharmaceutically acceptable salts thereof can be discovered together with other substances such as water and solvents (e.g., hydrates and solvates), or can be isolated.

In some embodiments, the preparation of a compound may involve adding an acid or a base, thereby affecting for example the catalysis of desired reactions or for example the formation of salt forms such as acid addition salts.

Examples of acids can be inorganic or organic acids, including but not limited to strong and weak acids. Some examples of acids include hydrochloric acid, hydrobromic acid, sulfuric acid, phosphoric acid, p-toluenesulfonic acid, 4-nitrobenzoic acid, methanesulfonic acid, benzenesulfonic acid, trifluoroacetic acid, and nitric acid. Some weak acids include but are not limited to acetic acid, propionic acid, butyric acid, benzoic acid, pyroglutamic acid, tartaric acid, valeric acid, hexanoic acid, heptanic acid, octanoic acid, nonanoic acid, and decanoic acid.

Examples of bases include lithium hydroxide, sodium hydroxide, potassium hydroxide, lithium carbonate, sodium carbonate, potassium carbonate, and sodium bicarbonate. Some examples of strong bases include but are not limited to hydroxides, alkoxides, metal amide compounds, metal hydrides, metal dialkylamides, and arylamines, wherein alkoxides include lithium salts, sodium salts, and potassium salts of methyl, ethyl, and tert-butyl oxides; metal amide compounds include sodium amide, potassium amide, and lithium amide; metal hydrides include sodium hydride, potassium hydride, and lithium hydride; and metal dialkylamides include lithium, sodium, and potassium salts of methyl, ethyl, n-propyl, isopropyl, n-butyl, tert-butyl, trimethylsilyl, and cyclohexyl substituted amides.

In some embodiments, the compounds and salts provided herein are substantially separated. By “substantially separated”, it means that a compound is at least partially or substantially separated from the environment in which it is formed or detected. Partial separation may include, for example, a composition that is rich in the compounds provided herein. Substantial separation may include compositions of the compounds or salts thereof provided herein comprising at least about 50%, at least about 60%, at least about 70%, at least about 80%, at least about 90%, at least about 95%, at least about 97%, or at least about 99% by weight. The methods for separating a compound from salts thereof are conventional in the art.

The phrase “pharmaceutically acceptable” as used herein refers to those compounds, materials, compositions, and/or dosage forms below that are suitable for use in contact with human and animal tissues within the scope of sound medical judgment without excessive toxicity, irritation, allergic reactions, or other issues or complications, and commensurate to reasonable benefits/risk ratios.

The present application also comprises pharmaceutically acceptable salts of the compounds described herein. As used herein, “pharmaceutically acceptable salts” refers to derivatives of the disclosed compounds, wherein a parent compound is modified by partially converting the existing acid or base into its salt forms. Examples of pharmaceutically acceptable salts include but are not limited to for example inorganic acid salts or organic acid salts of alkaline residues such as amine; and for example alkali metal salts or organic salts of acidic residues such as carboxylic acids. The pharmaceutically acceptable salts of the present application include for example, conventional non-toxic salts of parent compounds formed from non-toxic inorganic or organic acids, mainly including inorganic acid salts such as salts of sulfuric acid, nitric acid, hydrobromic acid, phosphoric acid, hydrochloric acid, boric acid, and sulphamic acid; or organic acids such as salts of acetic acid, propionic acid, butyric acid, camphoric acid, decanoic acid, hexanoic acid, octanoic acid, carbonic acid, cinnamic acid, hydroxyacetic acid, trifluoroacetic acid, adipic acid, alginic acid, 2-hydroxypropionic acid, 2-oxopropionic acid, stearic acid, lactic acid, citric acid, oxalic acid, malonic acid, succinic acid, pyroglutamic acid, ascorbic acid, aspartic acid, phenylacetic acid, glutamic acid, benzoic acid, salicylic acid, hydroxymaleic acid, palmitic acid, cinnamic acid, isobutyric acid, lauric acid, mandelic acid, maleic acid, fumaric acid, malic acid, tartaric acid, sulfanilic acid, 2-acetyloxybenzoic acid, 2-hydroxy-1,2,3-tricarballylic acid, octanedioic acid, gluconic acid, glucuronic acid, glutamic acid, glutaric acid, formic acid, fumaric acid, mucic acid, gentisic acid, pyruvic acid, salicylic acid, methanesulfonic acid, ethylsulfonic acid, benzene methanesulfonic acid, p-toluenesulfonic acid, cyclohexyl sulfinic acid, isethionic acid, ethanedisulfonic acid, 4-(fluorenemethoxycarbonylamino) butyric acid, dichloroacetic acid, 1,2-ethanedisulfonic acid, camphore-10 sulfonic acid, 2,4-dihydroxybenzoic acid, α-ketoglutaric acid, 1-hydroxy-2-naphthoic acid, p-acetamidobenzoic acid, 2-hydroxybenzoic acid, 4-amino-2-hydroxybenzoic acid, all-trans retinoic acid, and valproic acid. The pharmaceutically acceptable salts of the present application can be synthesized from parent compounds containing alkaline or acidic moieties using conventional chemical methods. Usually, such salts can be prepared by reacting the free acid or base form of these compounds with a stoichiometric amount of appropriate bases or acids in water, or organic solvents, or a mixture of both. Typically, non-aqueous media such as ether, ethyl acetate, alcohols (such as methanol, ethanol, isopropanol or butanol), or acetonitrile (MeCN) are preferred.

In some embodiments, a disease is a cancer. In some embodiments, the cancer is selected from the group consisting of colorectal cancer, endometrial cancer, brain cancer (for example, glioblastoma multiforme), melanoma, gastric cancer, breast cancer, ovarian cancer, pancreatic cancer, liver cancer, glioma, intracerebral tumor, kidney cancer, prostate cancer, bladder cancer, lung cancer, pancreatic cancer, ovarian cancer, skin cancer, epithelial cell cancer, nasopharyngeal cancer, epidermal cell cancer, cervical cancer, oral cancer, tongue cancer, human fibrosarcoma, multiple myeloma and hematological cancer. In some embodiments, the cancer includes solid tumors. In some embodiments, the cancer is selected from the group consisting of glioma, glioblastoma, non-small cell lung cancer, and hematological cancer.

In some embodiments, the cancer is the hematological cancer. In some embodiments, the hematological cancer is selected from the group consisting of leukemia and lymphoma. In some embodiments, the hematological cancer is selected from the group consisting of acute myeloid leukemia (AML), chronic myeloid leukemia, B-cell lymphoma, chronic lymphocytic leukemia (CLL), non Hodgkin's lymphoma, hairy cell leukemia, mantle cell lymphoma, Burkitt lymphoma, small lymphocytic lymphoma, follicular lymphoma, lymphoplasmacytic lymphoma, extranodal marginal zone lymphoma, activated B-cell like (ABC) diffuse large B cell lymphoma and germinal center B cell (GCB) diffuse large B cell lymphoma.

The compound described in the present disclosure can be used alone or in combination with other therapeutic agents for treating the diseases or disorders described in the present disclosure. The compound of the present disclosure is combined with other anti-tumor drugs. The anti-tumor drugs described include but are not limited to: cyclophosphamide, nitrogen mustard, melphalan, chlorambucil, carmustine, platinum metals such as carboplatin, cisplatin, oxaliplatin, camptothecin, irinotecan, daunorubicin, doxorubicin, bleomycin, plicamycin, paclitaxel, vinorelbine, docetaxel, doxorubicin, fluorouracil, methotrexate, cytarabine, gemcitabine, EGFR inhibitor, a VEGFR inhibitor, an ALK inhibitor, a BTK inhibitor, an mTOR inhibitor, and an HDAC inhibitor.

When used as a pharmaceutical drug, the compounds and salts provided herein can be administered in a form of pharmaceutical compositions. These compositions can be prepared as described herein or elsewhere, and can be administered through a variety of routes, depending on desired local or systemic treatments and an area to be treated. The administration can be local (including percutaneous, epidermal, ophthalmic, and mucosal administration including intranasal, vaginal, and rectal delivery), transpulmonary (for example, through inhalation or insufflation of powders or aerosols, including with the help of a sprayer; intratracheal or intranasal), oral, or parenteral. Parenteral administration includes intravenous, intraarterial, subcutaneous, intraperitoneal, intramuscular injection or infusion; or intracranial (e.g., Intrathecal or intraventricular administration). Parenteral administration can take the form of a single bolus dose, or for example, through a continuous perfusion pump.

In some embodiments, the compounds, salts, and pharmaceutical compositions provided herein are suitable for parenteral administration. In some embodiments, the compounds, salts, and pharmaceutical compositions provided herein are suitable for intravenous administration.

Pharmaceutical compositions and preparations for local administration may include transdermal patches, ointments, lotions, creams, gels, drops, suppositories, sprays, liquid preparations and powders. Conventional drug carriers, aqueous, powdered or oily matrices, thickeners and the like may be necessary or desirable.

Also provided is a pharmaceutical composition comprising the compounds provided herein or pharmaceutically acceptable salts thereof in combination with one or more pharmaceutically acceptable carriers (such as excipients) as active ingredients. When the composition provided herein is prepared, the active ingredients are usually mixed with an excipient so as to allow the active ingredients to be diluted or encapsulated in a carrier in the form of for example a capsule, anther sac, paper, or other containers through the excipient. When the excipient is used as a diluent, it can be a solid, semi-solid or liquid material that acts as a vehicle, a carrier, or a medium for the active ingredients. Therefore, the composition can take the following forms: tablets, pills, powders, lozenges, capsules, cachets, elixirs, suspensions, emulsions, solutions, syrups, aerosols (as solid or in liquid media), ointments, soft and hard gelatin capsules, suppositories, sterile injectable solutions, and sterile packaged powders.

Some examples of appropriate excipients include without limitation: lactose, glucose, sucrose, sorbitol, mannitol, starch, acacia gum, calcium phosphate, alginate, tragacanth gum, gelatin, calcium silicate, microcrystalline cellulose, polyvinylpyrrolidone, cellulose, water, syrup, and methylcellulose. The formulation may additionally include the following without limitation: lubricants such as talc, magnesium stearate, and mineral oil; wetting agents; emulsifiers and suspension aids; preservatives, such as methyl hydroxybenzoate and propyl hydroxybenzoate; sweeteners; flavouring agents; or combinations thereof.

The active ingredients can be effective over a wide range of doses and are typically administered in pharmaceutically effective amounts. However, it will be understood that the actual amount of the compound administered will usually be determined by physicians based on relevant circumstances, including conditions to be treated, chosen routes of administration, an actual compound administered, ages, weights and reactions of individual subjects, and the severity of subject's symptoms, etc.

The beneficial effect of the present disclosure lies in the drug design based on HDAC multi-targets, providing new chemical entities for the treatment of various cancers, thereby transforming the current cancer treatment methods.

### BRIEF DESCRIPTION OF THE DRAWINGS

FIG. 1 shows concentration-response curves of some compounds on acute myelogenous leukemia cells MV4-11 and HL60.

FIG. 2 shows concentration-response curves of some compounds on acute myelogenous leukemia cells PL21, KASUMI-1, MONO-MAC-1 and NB-4.

FIG. 3 shows concentration-response curves of CZ411 and FK866, and their mixed liquors on acute myelogenous leukemia cells MV4-11 and HL60.

FIG. 4 shows that NMN has a reversal effect on cell death caused by LEE18 and LEE12. NMN is nicotinamide mononucleotide.

FIG. 5 shows HCT116 tumor growth curves and tumor images. 5-FU: 5-fluorouracil, a traditional anti-tumor chemotherapy drug; Oxaliplatin, a third-generation platinum anticancer drug, is an anti-tumor chemotherapy drug. 5-FU+Oxaliplatin is positive control.

FIG. 6 shows a tumor growth curve. Panobinostat is a broad-spectrum HDAC inhibitor marketed as a positive control drug.

### DETAILED DESCRIPTION

The present disclosure will be described in more detail through specific embodiments. The following embodiments are provided for illustrative purposes and are not intended to limit the present disclosure in any way. Those skilled in the art will easily recognize a variety of non-critical parameters that can be changed or modified to achieve essentially the same results.

### General Materials and Methods

All reactions that are insensitive to air and moisture are carried out in an ambient atmosphere and subjected to magnetic stirring. Tetrahydrofuran is distilled from deep purple sodium benzophenone ketyl. Dry DMF, dry DMSO, dry acetonitrile, dry dichloromethane, dry toluene, and dry dioxane are purchased from Energy Chemical. All operations sensitive to air and moisture are carried out using oven-dried glassware in a nitrogen atmosphere.

Thin layer chromatography (TLC) is carried out with an EMD TLC plate pre-coated with a 250 μm thick silica gel 60F254 plate, and visualized through fluorescence quenching under UV light and KMnO₄ staining.

All deuterated solvents are purchased from Beijing J&K Scientific. NMR spectra are recorded on the following instrument: JEOL 400 spectrometer running at 400 MHz for ¹H and ¹³C acquisition. Using solvent resonance as the internal standard (¹H: CDCl₃, δ 7.26; DMSO-d₆, δ 2.50), (¹³C: CDCl₃, δ 77.16; DMSO-d₆, δ 39.52), the chemical shifts are reported in ppm. The data are reported as follows: s is singlet, d is doublet, t is triplet, q is quartet, and m is multiplet; coupling constant in Hz; Integral; Unless otherwise noted, carbon signals are unimodal.

The compounds of the present disclosure and their preparation methods are further elucidated and illustrated by the examples and preparation examples provided below. It should be understood that the scope of the following examples and preparation examples does not limit the scope of the present disclosure in any way.

The following synthesis routes describe the preparation of compounds of formulas II, III, IV, or V in the present disclosure, and all raw materials are prepared by the methods described in these routes, and methods well-known by those of ordinary skill in the field of organic chemistry, or commercially available. All the final compounds of the present disclosure are prepared by the methods described in these routes or by methods similar therewith, which are well-known to those of ordinary skill in the field of organic chemistry. All variable factors applied in these routes are as defined below or as defined in the claims.

The preparation of intermediate compounds of formulas II, III, IV or V in the present disclosure is as shown in routes 1 and 2, and each substituent is as defined in the summary of the present disclosure.

### Synthesis Route 1:

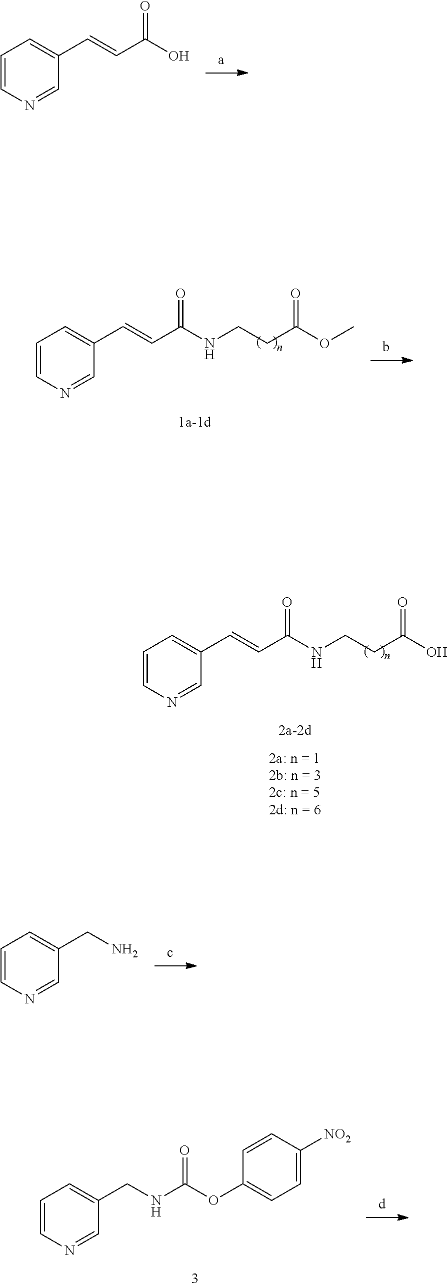

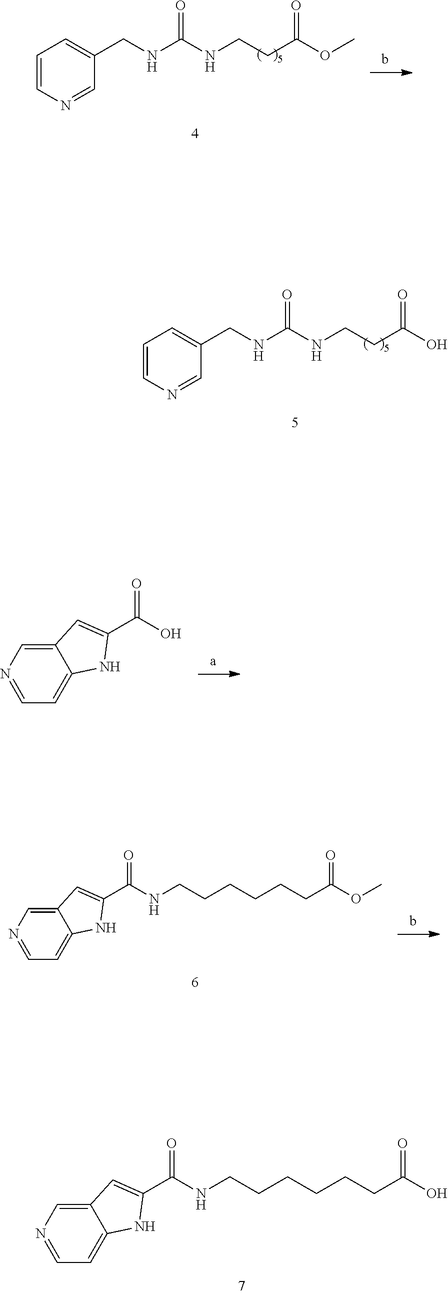

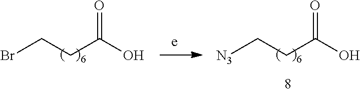

Reagents and conditions: (a) different amine, TBTU, TEA, DCM, yield 55%; (b) CH₃OH/H₂O, KOH, reflux, yield 80%; (c) 4-nitrophenyl chloroformate, TEA, DCM, yield 80%; (d) methyl 8-aminocaprylate hydrochlorid, TEA, DCM, yield 65%; (e) sodium azide, DMF, 80° C., yield 80%.

### Synthetic Route 2:

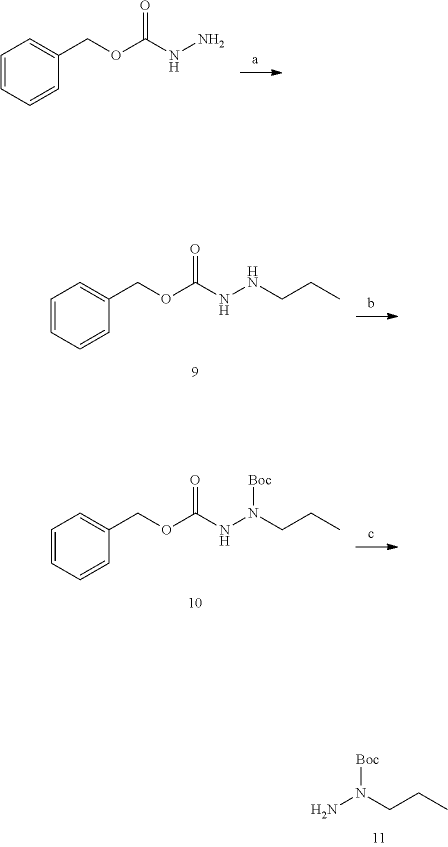 [SMILES: NNC(=O)OCc1ccccc1 ; CCCNNC(=O)OCc1ccccc1 ; CCCN(NC(=O)OCc1ccccc1)C(=O)OC(C)(C)C ; CCCN(N)C(=O)OC(C)(C)C]

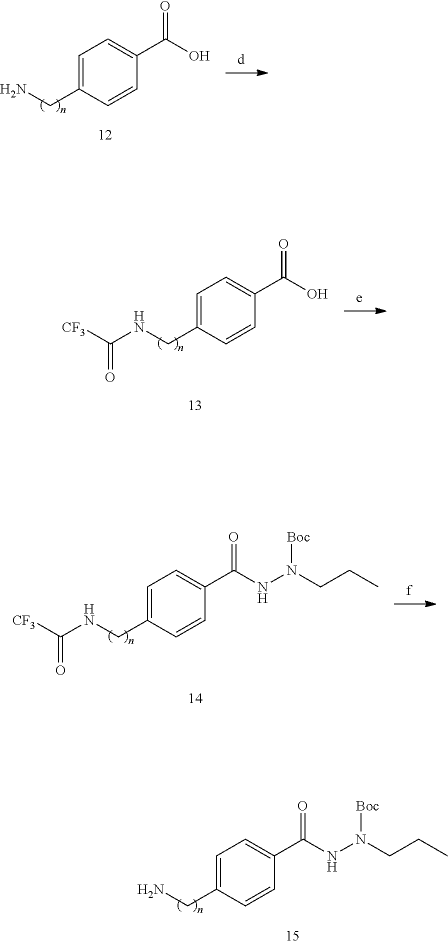

Reagents and conditions: (a) propionaldehyde, MeOH, yield 98%; NaBH₃CN, MeOH, concentrated hydrochloric acid, methyl orange, yield 60%; (b) (Boc)₂O, triethylamine, EtOH, yield 85%; (c) Pd/C, H₂, MeOH, yield 85%; (d) trifluoroacetic anhydride, DCM, yield 85%; (e) EDCI, HOBt, TEA, DCM, yield 55%; (f) Na₂CO₃, MeOH, yield 70%.

The preparation of compounds of formulas II, III, IV or V in the present disclosure is as shown in routes 3, 4, 5 and 6, and each substituent is as defined in the summary of the present disclosure.

### Route 3:

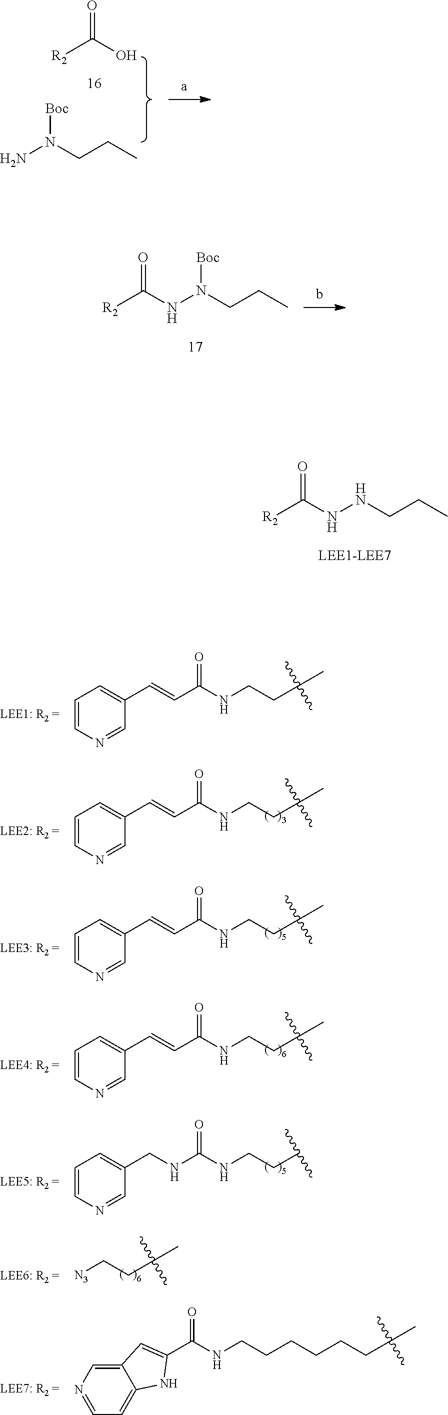

Reagents and conditions: (a) TBTU, TEA, DCM, yield 55%; (b) TFA, DCM, triethylamine, yield 85%.

### Route 4:

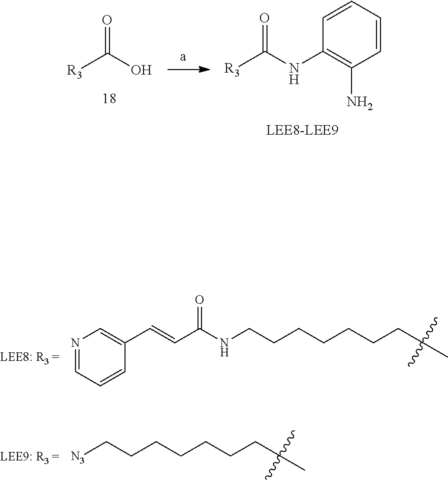

Reagents and conditions: (a) TBTU, TEA, DCM, yield 55%.

### Route 5:

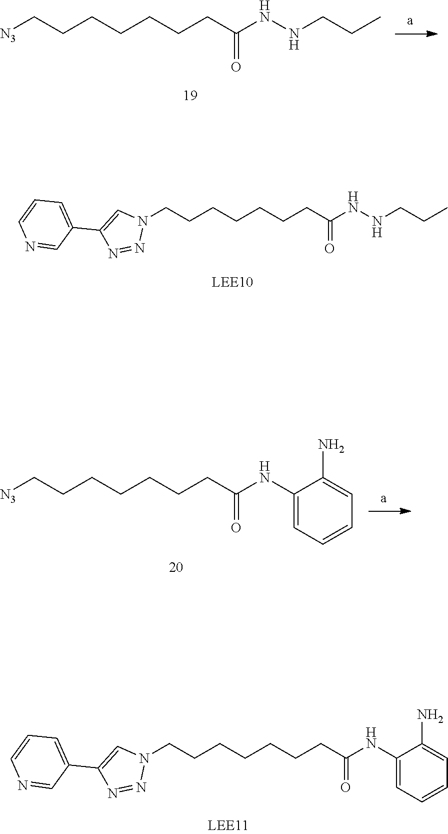 [SMILES: CCCNNC(=O)CCCCCCCN=[N+]=[N-] ; CCCNNC(=O)CCCCCCCn1cc(-c2cccnc2)nn1 ; [N-]=[N+]=NCCCCCCCC(=O)Nc1ccccc1N ; Nc1ccccc1NC(=O)CCCCCCCn1cc(-c2cccnc2)nn1]

Reagents and conditions: (a) 3-ethynylpyridine, sodium ascorbate, CuSO₄, THF. H₂O, yield 85%.

### Route 6:

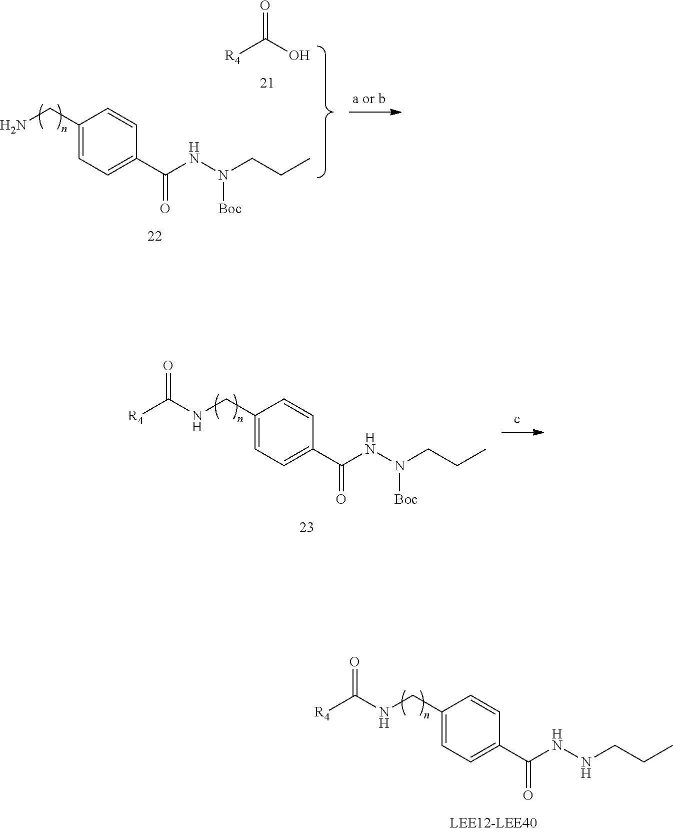

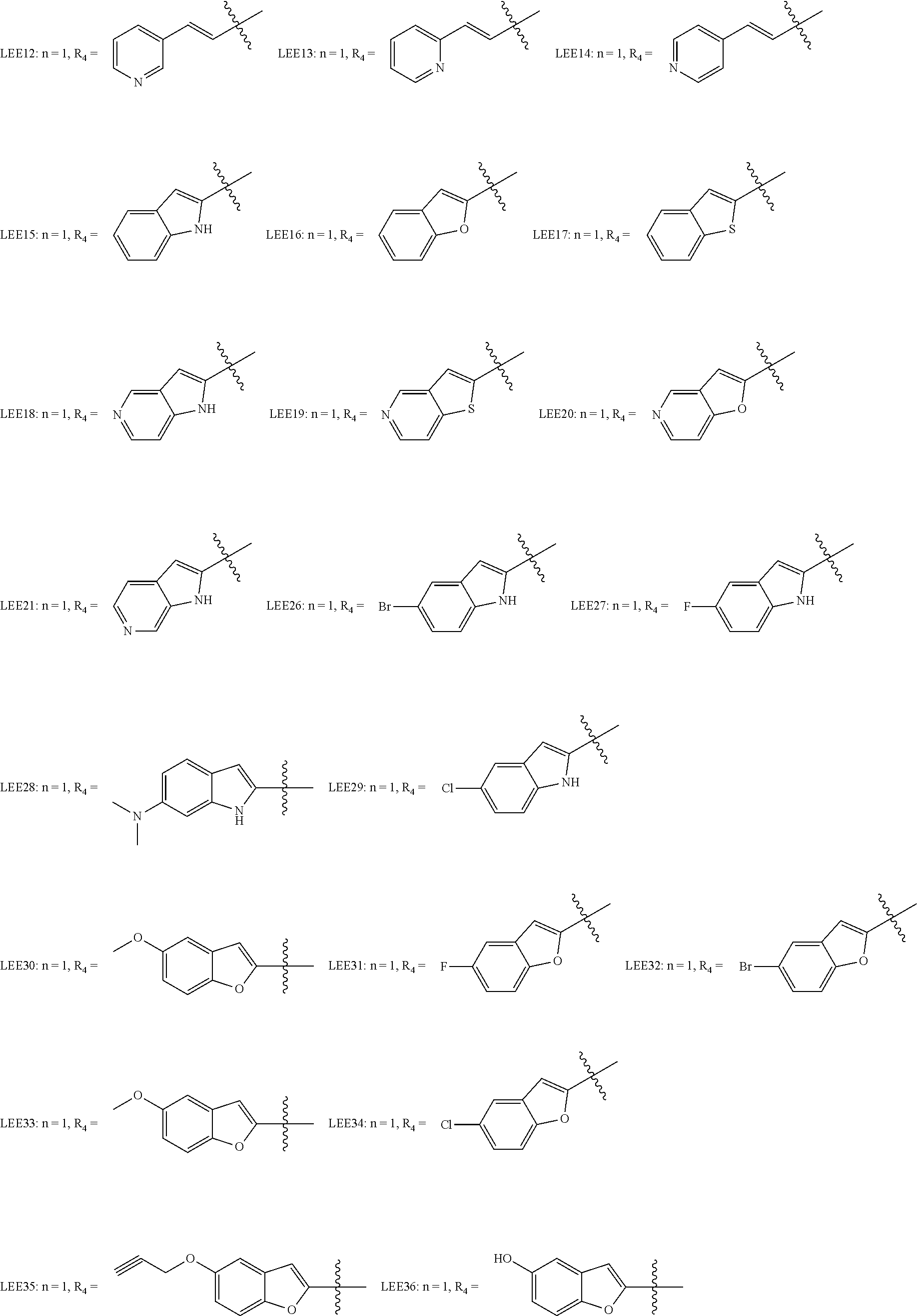

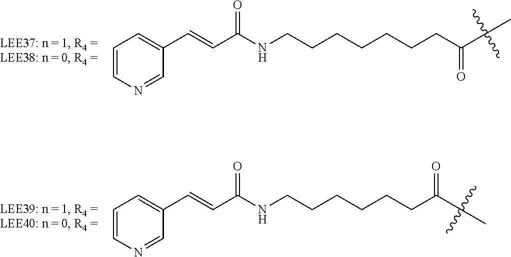 [SMILES: *C(=O)CCCCCCCNC(=O)/C=C/c1cccnc1 ; *C(=O)CCCCCCNC(=O)/C=C/c1cccnc1]

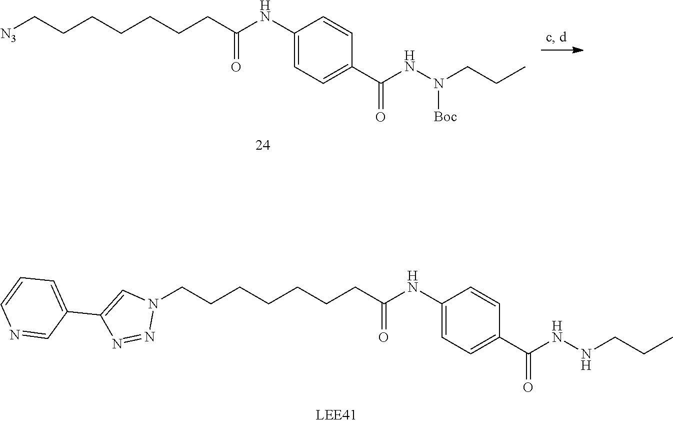 [SMILES: CCCN(NC(=O)c1ccc(NC(=O)CCCCCCCN=[N+]=[N-])cc1)C(=O)OC(C)(C)C ; CCCNNC(=O)c1ccc(NC(=O)CCCCCCCn2cc(-c3cccnc3)nn2)cc1]

Reagents and conditions: (a) TBTU, TEA, DCM, yield 55%; (b) EDCI, HOBt, triethylamine, DCM, yield 50%; (c) TFA, DCM, triethylamine, yield 85%; (d) 3-ethynylpyridine, sodium ascorbate, CuSO₄, THF, H₂O, yield 85%.

### SPECIFIC EMBODIMENTS

Next, the present disclosure will be explained in detail through specific examples, but the use and purpose of these exemplary embodiments are only used for exemplifying the present disclosure and do not constitute any form of limitation on the actual scope of protection of the present disclosure, rather than limiting the scope of protection of the present disclosure thereto.

### Preparation of Compounds in Route 1

### Example 1

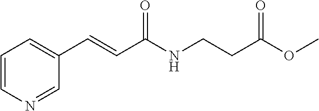 [SMILES: COC(=O)CCNC(=O)/C=C/c1cccnc1]

Preparation of methyl (E)-3-(3-(pyridin-3-yl)acrylamido) propionate (1a): 3-(pyridin-3-yl)acrylic acid (0.44 g, 3 mmol) was dissolved in 20 mL of dichloromethane, and 2-(1H-benzotriazol-1-yl)-1,1,3,3-tetramethylurea tetrafluoroborate (TBTU, 1.05 g, 3.6 mmol) and TEA (0.6 mL, 4.5 mmol) were added in an ice bath. After 30 minutes, methyl 3-aminopropionate hydrochloride (0.46 g, 3.3 mmol) was added, then 0.6 mL of TEA was added and the above materials reacted overnight. The reaction product was washed with saturated NaHCO₃ (2×30 mL) and saturated brine (2×30 mL) and dried over MgSO₄. After the solvent was completely evaporated, the product obtained after evaporation was purified by flash chromatography to obtain compound 1a as a white solid powder (0.35 g, 51%). ¹H NMR (600 MHz, DMSO-d₆) δ 8.75 (d, J=2.3 Hz, 1H), 8.55 (dd, J=4.8, 1.6 Hz, 1H), 8.28 (t, J=5.7 Hz, 1H), 7.98 (dt, J=8.0, 2.0 Hz, 1H), 7.49-7.42 (m, 2H), 6.73 (d, J=15.9 Hz, 1H), 3.62 (s, 3H), 3.42 (td, J=6.8, 5.6 Hz, 2H), 2.55 (t, J=6.8 Hz, 2H). ESI-MS m/z: 234.87 [M+H]⁺.

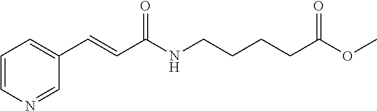 [SMILES: COC(=O)CCCCNC(=O)/C=C/c1cccnc1]

Preparation of methyl (E)-5-(3-(pyridin-3-yl)acrylamido) pentanoate (1b): the synthesis method of 1a was used, and 3-(pyridin-3-yl)acrylic acid and methyl 5-aminopentanoate hydrochloride were used as raw material to obtain compound 1b as a white solid, with a yield of 55%. ¹H NMR (600 MHz, DMSO-d₆) δ 8.75 (d, J=2.3 Hz, 1H), 8.55 (dd, J=4.8, 1.6 Hz, 1H), 8.18 (t, J=5.8 Hz, 1H), 7.98 (dt, J=7.9, 2.0 Hz, 1H), 7.48-7.42 (m, 2H), 6.72 (d, J=15.9 Hz, 1H), 3.59 (s, 3H), 3.22-3.16 (m, 2H), 2.34 (t, J=7.4 Hz, 2H), 1.60-1.52 (m, 2H), 1.52-1.43 (m, 2H). ESI-MS m/z: 248.97 [M+H]⁺.

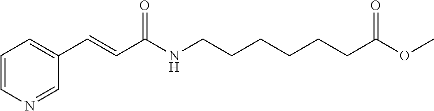 [SMILES: COC(=O)CCCCCCNC(=O)/C=C/c1cccnc1]

Preparation of methyl (E)-7-(3-(pyridin-3-yl)acrylamido) heptanoate (1c): the synthesis method of 1a was used, and 3-(pyridin-3-yl)acrylic acid and methyl 7-aminoheptanoate hydrochloride were used as raw material to obtain compound 1c as a white solid, with a yield of 53%. ¹H NMR (600 MHz, DMSO-d₆) δ 8.75 (d, J=2.3 Hz, 1H), 8.55 (dd, J=4.7, 1.6 Hz, 1H), 8.14 (t, J=5.7 Hz, 1H), 7.97 (dt, J=7.9, 2.0 Hz, 1H), 7.50-7.41 (m, 2H), 6.72 (d, J=15.9 Hz, 1H), 3.58 (s, 3H), 3.17 (td, J=7.0, 5.7 Hz, 2H), 2.34-2.26 (m, 2H), 1.53 (qd, J=7.4, 3.2 Hz, 2H), 1.45 (p, J=7.4 Hz, 2H), 1.30 (h, J=4.5, 3.5 Hz, 4H). ESI-MS m/z: 290.86 [M+H]⁺.

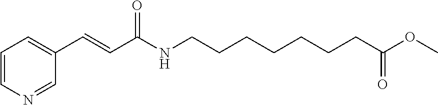 [SMILES: COC(=O)CCCCCCCNC(=O)/C=C/c1cccnc1]

Preparation of methyl (E)-7-(3-(pyridin-3-yl)acrylamido) octanoate (1d): the synthesis method of 1a was used, and 3-(pyridin-3-yl)acrylic acid and methyl 8-aminooctanoate hydrochloride were used as raw material to obtain compound 1d as a white solid, with a yield of 53%. ¹H NMR (600 MHz, DMSO-d₆) δ 8.75 (d, J=2.3 Hz, 1H), 8.55 (dd, J=4.7, 1.6 Hz, 1H), 8.14 (t, J=5.7 Hz, 1H), 7.97 (dt, J=7.9, 2.0 Hz, 1H), 7.47-7.42 (m, 2H), 6.72 (d, J=15.9 Hz, 1H), 3.58 (s, 3H), 3.20-3.14 (m, 2H), 2.30 (t, J=7.4 Hz, 2H), 1.56-1.50 (m, 2H), 1.49-1.40 (m, 2H), 1.31-1.24 (m, 7H).

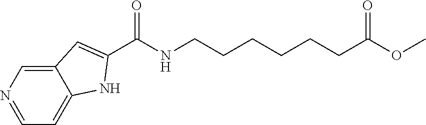 [SMILES: COC(=O)CCCCCCNC(=O)c1cc2cnccc2[nH]1]

Preparation of methyl 7-(3H pyrrolo[3,2-c]pyridin-2-carboxamide) heptanate (6): the synthesis method of 1a was used, and 1H-pyrrolo[3,2-c]pyridin-2-carboxylic acid and methyl 7-aminoheptanoate hydrochloride were used as raw material to obtain compound 6 as a white solid, with a yield of 55%. ¹H NMR (600 MHz, DMSO-d₆) δ 8.75 (d, J=2.3 Hz, 1H), 8.55 (dd, J=4.7, 1.6 Hz, 1H), 8.14 (t, J=5.7 Hz, 1H), 7.97 (dt, J=7.9, 2.0 Hz, 1H), 7.47-7.42 (m, 2H), 6.72 (d, J=15.9 Hz, 1H), 3.58 (s, 3H), 3.17 (td, J=7.0, 5.7 Hz, 2H), 2.30 (t, J=7.4 Hz, 2H), 1.52 (t, J=7.2 Hz, 2H), 1.45 (q, J=7.1 Hz, 2H), 1.31-1.24 (m, 7H). ESI-MS m/z: 304.89 [M+H]⁺.

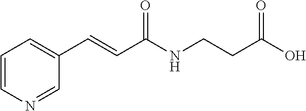 [SMILES: O=C(O)CCNC(=O)/C=C/c1cccnc1]

Preparation of (E)-3-(3-(pyridin-3-yl)acrylamido) propionic acid (2a): compound 1a (0.35 g, 1.5 mmol) was dissolved in 5 mL of methanol, and then 2 mL of 3M KOH aqueous solution was added. The mixture was refluxed at 85° C. for 2 h. After the reaction was completed, MeOH was evaporated under vacuum. The residue was acidified with 1 N HCl to pH 5-6, and then filtered. The precipitate was corresponding acidic compound 2a as a white solid (0.28 g, 85%). The raw materials were directly used for the next step without further purification. ESI-MS m/z: 219.15 [M−H]⁻.

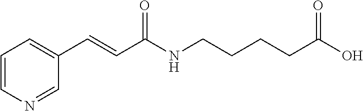 [SMILES: O=C(O)CCCCNC(=O)/C=C/c1cccnc1]

Preparation of (E)-5-(3-(pyridin-3-yl)acrylamido) pentanoic acid (2b): the synthesis method of 2a was used to obtain compound 2b as a white solid, with a yield of 88%. ¹H NMR (400 MHz, DMSO-d₆) δ 8.39 (d, J=13.3 Hz, 2H), 7.59 (d, J=7.9 Hz, 1H), 7.29 (dd, J=8.1, 4.3 Hz, 1H), 6.38 (t, J=6.0 Hz, 1H), 5.98 (t, J=5.8 Hz, 1H), 4.17 (d, J=5.9 Hz, 2H), 2.97-2.92 (m, 2H), 2.14 (t, J=7.3 Hz, 2H), 1.45-1.24 (m, 8H). ESI-MS m/z: 278. 18 [M−H]⁻.

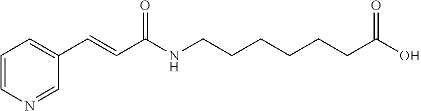 [SMILES: O=C(O)CCCCCCNC(=O)/C=C/c1cccnc1]

Preparation of (E)-7-(3-(pyridin-3-yl)acrylamido) heptanoic acid (2c): the synthesis method of 2a was used and compound LL433 was used as a raw material to obtain compound 2c as a white solid, with a yield of 55%. ¹H NMR (400 MHz, DMSO-d₆) δ 8.97 (d, J=2.1 Hz, 1H), 8.73 (dd, J=5.4, 1.4 Hz, 1H), 8.46 (dt, J=8.2, 1.8 Hz, 1H), 8.29 (t, J=5.7 Hz, 1H), 7.85 (dd, J=8.1, 5.4 Hz, 1H), 7.50 (d, J=15.9 Hz, 1H), 6.85 (d, J=15.9 Hz, 1H), 3.14 (q, J=6.6 Hz, 2H), 2.26 (t, J=7.4 Hz, 2H), 1.45 (dt, J=28.5, 7.1 Hz, 4H), 1.25 (p, J=3.6 Hz, 4H). ESI-MS m/z: 275. 24 [M−H]⁻.

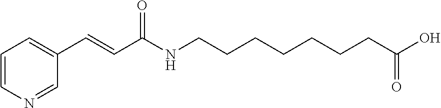 [SMILES: O=C(O)CCCCCCCNC(=O)/C=C/c1cccnc1]

Preparation of (E)-8-(3-(pyridin-3-yl)acrylamido) octanoic acid (2d): the synthesis method of 2a was used and compound 1d was used as a raw material to obtain compound 2d as a white solid, with a yield of 87%. ¹H NMR (400 MHz, DMSO-d₆) δ 8.97 (d, J=2.1 Hz, 1H), 8.73 (dd, J=5.4, 1.4 Hz, 1H), 8.46 (dt, J=8.2, 1.8 Hz, 1H), 8.29 (t, J=5.7 Hz, 1H), 7.85 (dd, J=8.1, 5.4 Hz, 1H), 7.50 (d, J=15.9 Hz, 1H), 6.85 (d, J=15.9 Hz, 1H), 3.14 (q, J=6.6 Hz, 2H), 2.26 (t, J=7.4 Hz, 2H), 1.45 (dt, J=28.5, 7.1 Hz, 4H), 1.25 (p, J=3.6 Hz, 4H). ESI-MS m/z: 275. 24 [M−H]⁻.

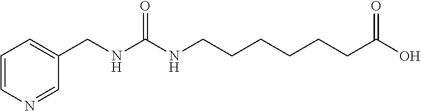 [SMILES: O=C(O)CCCCCCNC(=O)NCc1cccnc1]

Preparation of 7-(3-(pyridin-3-ylmethyl) ureido) heptanoic acid (5): the synthesis method of 2a was used and compound 4 was used as a raw material to obtain compound 5 as a white solid, with a yield of 85%. ¹H NMR (400 MHz, DMSO-d₆) δ 8.39 (d, J=13.3 Hz, 2H), 7.59 (d, J=7.9 Hz, 1H), 7.29 (dd, J=8.1, 4.3 Hz, 1H), 6.38 (t, J=6.0 Hz, 1H), 5.98 (t, J=5.8 Hz, 1H), 4.17 (d, J=5.9 Hz, 2H), 2.97-2.92 (m, 2H), 2.14 (t, J=7.3 Hz, 2H), 1.45-1.24 (m, 8H). ESI-MS m/z: 278. 18 [M−H]⁻.

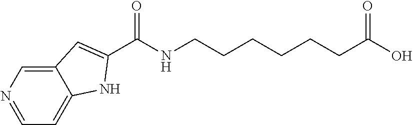 [SMILES: O=C(O)CCCCCCNC(=O)c1cc2cnccc2[nH]1]

7-(1H-pyrrolo [3,2-c] pyridin-2-carboxamide) heptanoic acid (7). The synthesis method of 2a was used and compound 6 was used as a raw material to obtain compound 7 as a white solid, with a yield of 85%. ESI-MS m/z: 288. 21 [M−H]⁻.

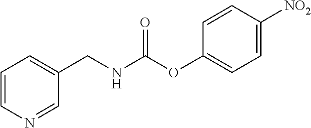 [SMILES: O=C(NCc1cccnc1)Oc1ccc([N+](=O)[O-])cc1]

Preparation of 4-nitrophenyl (pyridin-3-ylmethyl) carbamate (3): 3-pyridinemethanimine (0.22 g, 2 mmol) and triethylamine (0.43 g, 2 mmol) were dissolved in DCM, then 4-nitrophenyl chloroformate (0.61 g, 3 mmol) was added, and the above materials were stirred at room temperature for 5 hours. After the reaction was completed, the reaction product was washed with brine three times and dried over Na₂SO₄. The solvent was completely evaporated to obtain compound 3 as a white solid powder (0.07 g, 70% yield). ¹H NMR (400 MHz, DMSO-d₆) δ 8.61 (t, J=6.1 Hz, 1H), 8.52 (s, 1H), 8.46 (d, J=4.8 Hz, 1H), 8.26-8.19 (m, 2H), 7.72 (d, J=8.0 Hz, 1H), 7.43-7.38 (m, 2H), 7.37-7.34 (m, 1H), 4.31 (d, J=6.0 Hz, 2H). ESI-MS m/z: 273.84 [M+H]⁺.

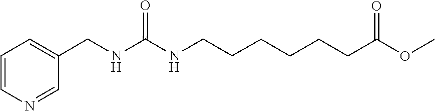 [SMILES: COC(=O)CCCCCCNC(=O)NCc1cccnc1]

Preparation of methyl 7-(3-(pyridin-3-ylmethyl) ureido) heptanoate (4): to a solution of methyl 7-aminoheptanoate hydrochloride (0.19 g, 1 mmol) in dichloromethane were added triethylamine (0.12 g, 1.2 mmol) and compound 3 (0.33 g, 1.2 mmol) at 0° C. The above materials reacted at room temperature for 2 hours. After the reaction was completed, the reaction product was washed with brine three times and dried over Na₂SO₄. After the solvent was completely evaporated, the product obtained after evaporation was purified by flash chromatography to obtain compound 4 as a white solid powder (0.2 g, 70% yield). ¹H NMR (400 MHz, DMSO-d₆) δ 8.42 (s, 1H), 8.39 (d, J=4.8 Hz, 1H), 7.60 (d, J=7.8 Hz, 1H), 7.29 (dd, J=7.8, 4.8 Hz, 1H), 6.31 (t, J=6.0 Hz, 1H), 5.92 (t, J=5.7 Hz, 1H), 4.17 (d, J=6.0 Hz, 2H), 3.54 (s, 3H), 2.94 (q, J=6.5 Hz, 2H), 2.24 (t, J=7.4 Hz, 2H), 1.47 (p, J=7.2 Hz, 2H), 1.31 (p, J=6.9 Hz, 2H), 1.21 (h, J=3.8, 3.0 Hz, 4H). ESI-MS m/z: 293.92 [M+H]⁺.

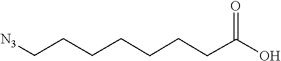 [SMILES: [N-]=[N+]=NCCCCCCCC(=O)O]

Preparation of 8-azido-octanoic acid (8): 8-bromooctanoic acid (0.33 g, 1.5 mmol) was dissolved in DMF, then NaN3 (0.15 g, 2.25 mmol) was added, and the above materials reacted overnight at 80° C. 25 mL of DCM was added to the solution, the solvent was washed with water 5 times and dried over Na2SO4. After the solvent was completely evaporated, compound 8 as a colorless oil (0.27 g, 95% yield) was obtained. ¹H NMR (400 MHz, DMSO-d₆) δ 2.15 (t, J=7.4 Hz, 2H), 1.46 (dp, J=14.4, 7.1 Hz, 4H), 1.28-1.19 (m, 6H). ESI-MS m/z: 184. 15 [M−H]⁻.

### Preparation of Compounds in Route 2

### Example 2

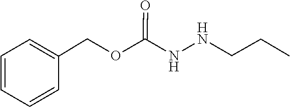

Preparation of benzyl 2-propylhydrazin-1-carboxylate (9): benzyl hydrazinecarboxylate (1.66 g, 10 mmol) was dissolved in 50 mL of methanol, then propionaldehyde (0.61 g, 10.5 mmol) was added, and the above materials reacted at room temperature for 2 hours. After the reaction was completed, methanol was removed. The obtained solid was dissolved in 30 mL of methanol, then NaBH₃CN (1.2 g, 20 mmol) and 2 drops of concentrated HCl/MeOH (v: v=1:1) solution were added, and the above materials reacted overnight. After the reaction was completed, the solvent was completely evaporated, and then a crude product was purified by flash chromatography to obtain compound 9 as a white solid powder (1.2 g, 60%). ¹H NMR (400 MHz, DMSO-d₆) δ 8.54 (s, 1H), 7.36-7.23 (m, 5H), 4.99 (s, 2H), 4.46 (s, 1H), 2.61 (t, J=7.1 Hz, 2H), 1.32 (h, J=7.3 Hz, 2H), 0.81 (t, J=7.4 Hz, 3H). ESI-MS m/z: 108.92 [M+H]⁺.

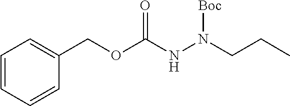 [SMILES: CCCN(NC(=O)OCc1ccccc1)C(=O)OC(C)(C)C]

Preparation of 2-benzyl 1-(tert-butyl) 1-propylhydrazine-1,2-dicarboxylate (10): the compound 9 (1 g, 5 mmol) was dissolved in 50 mL of anhydrous dichloromethane, and then triethylamine (1.5 g, 15 mmol) and (Boc)₂O (0.22 g, 10 mmol) were added. After reacting at room temperature for 2 hours, the solution was washed with a 1 M citric acid aqueous solution (3×100 mL and brine (3×100 mL) and then dried over MgSO₄, and the solvent was completely evaporated to obtain compound 10 as a white solid (1.3 g, 85%). ¹H NMR (400 MHz, DMSO-d₆) δ 9.45 (s, 1H), 7.39-7.22 (m, 5H), 5.05 (s, 2H), 3.24 (s, 2H), 1.44-1.27 (m, 11H), 0.79 (t, J=7.3 Hz, 3H). ESI-MS m/z: 308.86 [M+H]⁺.

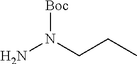

Preparation of tert-butyl 1-propylhydrazin-1-carboxylate (11): compound 10 (0.6 g, 2 mmol) was dissolved in methanol and Pd/C (0.06 g) was added. Then the above materials reacted in hydrogen at room temperature for 4 hours. After the reaction was completed, Pd/C was removed by filtration, and the filtrate was completely evaporated to obtain compound 11 as a colorless oil (0.25 g, 72%). ¹H NMR (400 MHz, DMSO-d₆) δ 4.37 (s, 1H), 3.16 (t, J=7.0 Hz, 2H), 1.45 (h, J=14.3, 7.2 Hz, 2H), 1.36 (s, 9H), 0.76 (t, J=7.4 Hz, 3H). ESI-MS m/z: 174.87 [M+H]⁺.

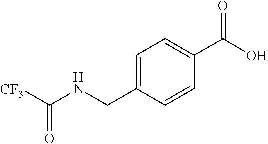 [SMILES: O=C(O)c1ccc(CNC(=O)C(F)(F)F)cc1]

Preparation of 4-((2,2,2-trifluoroacetamido)methyl) benzoic acid (13a): 10 mL of trifluoroacetic anhydride was slowly added to 4-(aminomethyl) benzoic acid (3.0 g, 20 mmol) in an ice bath, and then reacted for 2 hours at room temperature after dropwise addition. After the reaction was completed, 100 mL of ice water was added to quench the reaction, then the reaction was filtered, and a filter cake was dried to obtain compound 13a (4.43 g, 95%). ¹H NMR (400 MHz, DMSO-d₆) δ 10.05 (t, J=6.0 Hz, 1H), 7.89 (d, J=6.4 Hz, 2H), 7.35 (d, J=8.0 Hz, 2H), 4.43 (d, J=6.0 Hz, 2H). ESI-MS m/z: 246. 16 [M−H]⁻.

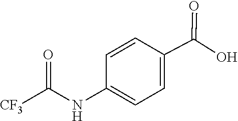 [SMILES: O=C(O)c1ccc(NC(=O)C(F)(F)F)cc1]

Preparation of 4-(2,2,2-trifluoroacetamido) methyl) benzoic acid (13b): the synthesis method of 13a was used, and 4-aminobenzoic acid and trifluoroacetic anhydride were used as raw materials to obtain compound 13b as a white solid, with a yield of 98%. ¹H NMR (400 MHz DMSO-d₆) δ 12.94 (s, 1H), 10.07 (s, 1H), 7.94 (d, J=8.2 Hz, 2H), 7.40 (d, J=8.2 Hz, 2H), 4.47 (d, J=6.0 Hz, 2H). ESI-MS m/z: 232.12 [M−H]⁻.

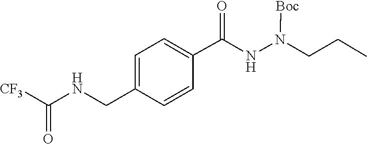 [SMILES: CCCN(NC(=O)c1ccc(CNC(=O)C(F)(F)F)cc1)C(=O)OC(C)(C)C]

Preparation of tert-butyl 1-propyl-2-(4-((2,2,2-trifluoroacetamido)methyl)benzoyl)hydrazine-1-carboxylate (14a): compound 13a (0.7 g, 3 mmol) was dissolved in 20 mL of dichloromethane, 1-ethyl-3-(3-dimethylaminopropyl) carbodiimide hydrochloride (EDCI·HCl, 0.7 g, 3.6 mmol), 1-hydroxybenzotriazole (HOBt, 0.44 g, 3.6 mmol) and triethylamine (0.6 mL, 4.5 mmol) were added in an ice bath. After 30 minutes, compound 11 (0.57 g, 3.3 mmol) was added and the above materials reacted overnight. The reaction solution was washed with saturated NaHCO₃ (2×30 mL) and brine (2×30 mL), and organic phases were combined and dried over anhydrous magnesium sulfate. After the solvent was completely evaporated, the obtained reaction product was purified by flash chromatography to obtain compound 14a as a white solid powder (0.66 g, 55% yield). ¹H NMR (400 MHz, DMSO-d₆) δ 10.47 (s, 1H), 10.05 (t, J=6.1 Hz, 1H), 7.78 (dd, J=16.9, 7.8 Hz, 2H), 7.39-7.29 (m, 2H), 4.41 (d, J=5.9 Hz, 2H), 3.34 (s, 2H), 1.47-1.27 (m, 11H), 0.84 (t, J=7.1 Hz, 3H). ESI-MS m/z: 403.89 [M+H]⁺.

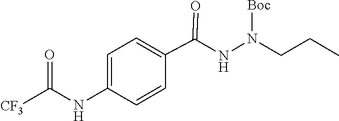 [SMILES: CCCN(NC(=O)c1ccc(NC(=O)C(F)(F)F)cc1)C(=O)OC(C)(C)C]

Preparation of tert-butyl 1-propyl-2-(4-(2,2,2-trifluoroacetamido) benzoyl) hydrazine-1-carboxylate (14b): the synthesis method of 14a was used and compounds 13b and 11 were used as raw materials to obtain compound 14b as a white solid, with a yield of 50%. ¹H NMR (400 MHz, DMSO-d₆) δ 11.46 (s, 1H), 10.49 (s, 1H), 7.85 (dd, J=14.7, 8.6 Hz, 2H), 7.75 (d, J=8.4 Hz, 2H), 1.55-1.43 (m, 2H), 1.40-1.29 (m, 9H), 0.84 (t, J=6.9 Hz, 3H). ESI-MS m/z: 389.91 [M+H]⁺.

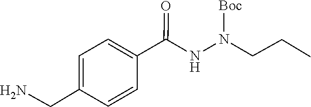 [SMILES: CCCN(NC(=O)c1ccc(CN)cc1)C(=O)OC(C)(C)C]

Preparation of tert-butyl 2-(4-(aminomethyl)benzoyl)-1-propylhydrazin-1-carboxylate (15a): compound 14a (1.2 g, 3 mmol) was dissolved in 20 mL of methanol/water (v: v=1:1) solution, then K₂CO₃ (1.24 g, 9 mmol) was added and the above materials reacted overnight at room temperature. After methanol was completely evaporated, the reaction solution was extracted with ethyl acetate (2×20 mL), organic phases were combined, washed with brine (2×30 mL) and dried over anhydrous Na₂SO₄. After filtration, the solvent was completely evaporated to obtain compound 15a (0.74 g, 80%). ¹H NMR (400 MHz, DMSO-d₆) δ 10.42 (s, 1H), 7.74 (dd, J=16.8, 7.9 Hz, 2H), 7.39 (d, J=8.0 Hz, 2H), 3.72 (s, 2H), 1.47 (p, J=7.3 Hz, 3H), 1.39-1.28 (m, 9H), 0.83 (t, J=7.0 Hz, 4H). ESI-MS m/z: 307.94 [M+H]⁺.

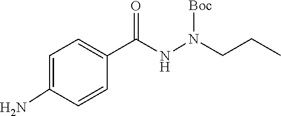 [SMILES: CCCN(NC(=O)c1ccc(N)cc1)C(=O)OC(C)(C)C]

Preparation of tert-butyl 2-(4-aminobenzoyl)-1-propylhydrazin-1-carboxylate (15b): the synthesis method of 15a was used and compound 14b was used as a raw material to obtain compound 15b as a white solid, with a yield of 82%. ¹H NMR (400 MHz, DMSO-d₆) δ 10.00 (s, 1H), 10.00 (s, OH), 7.58-7.49 (m, 2H), 6.54-6.46 (m, 2H), 5.68 (d, J=4.7 Hz, 2H), 3.32-3.25 (m, 2H), 1.46 (h, J=7.5 Hz, 2H), 1.38-1.27 (m, 9H), 0.82 (t, J=7.1 Hz, 3H). ESI-MS m/z: 293.87 [M+H]⁺.

### Preparation of Compounds in Route 3

### Example 3

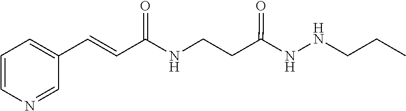 [SMILES: CCCNNC(=O)CCNC(=O)/C=C/c1cccnc1]

Preparation of (E)-N-(3-oxo-3-(2-propylhydrazino) propyl)-3-(pyridin-3-yl) acrylamide (LEE1): compound 2b (0.53 g, 3 mmol) was dissolved in 15 mL of dichloromethane, and O-benzotriazole-N,N,N′,N′-tetramethylurea tetrafluoroborate (TBTU, 1.05 g, 3.6 mmol) and triethylamine (0.6 mL, 4.5 mmol) were added in an ice bath. After reacting for 30 minutes, compound 11 (0.57 g, 3.3 mmol) was added and the above materials reacted overnight at room temperature. The reaction solution was washed with saturated NaHCO₃ (2×30 mL) and saturated brine (2×30 mL) and dried over MgSO₄. After filtration, the solvent was completely evaporated, and then the obtained product was purified by flash chromatography to obtain a white solid powder (0.35 g, 54%).

The product (0.30 g, 0.8 mmol) of the previous step was dissolved in 15 mL of dichloromethane, then trifluoroacetic acid (TFA, 0.11 g, 1.0 mmol) was added and the above materials reacted overnight. After the reaction was completed, TEA (0.10 g, 1.0 mmol) was added to adjust the pH to 8. The solution was washed with brine and dried over Na₂SO₄. After filtration, the solvent was completely evaporated, and then the obtained product was then purified by flash chromatography to obtain compound LEE1 as a white solid powder (0.18 g, 85%). ¹H NMR (400 MHz, DMSO-d₆) δ 9.32 (s, 1H), 8.75 (d, J=2.2 Hz, 1H), 8.55 (dd, J=4.7, 1.6 Hz, 1H), 8.21 (t, J=5.8 Hz, 1H), 7.97 (dt, J=8.0, 2.0 Hz, 1H), 7.50-7.40 (m, 2H), 6.74 (d, J=15.9 Hz, 1H), 4.79 (s, 1H), 3.39 (q, J=6.6 Hz, 2H), 2.60 (dt, J=18.8, 6.8 Hz, 2H), 2.27 (t, J=7.0 Hz, 2H), 1.38 (h, J=7.3 Hz, 2H), 0.85 (t, J=7.4 Hz, 3H). ¹³C NMR (101 MHz, DMSO-d₆) δ 169.49, 165.00, 150.57, 149.55, 135.71, 134.36, 131.18, 124.65, 124.43, 53.50, 35.92, 34.05, 21.21, 12.02. ESI-MS m/z: 276.88 [M+H]⁺.

### Example 4

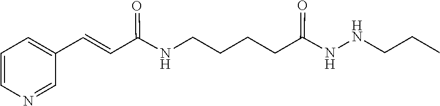 [SMILES: CCCNNC(=O)CCCCNC(=O)/C=C/c1cccnc1]

Preparation of (E)-N-(5-oxo-5-(2-propylhydrazino)pentyl)-3-(pyridin-3-yl) acrylamide (LEE2): the synthesis method of LEE1 was used and compound 11 and compound 2b were used as raw materials to obtain compound LEE2 as a white solid, with a yield of 55%. ¹H NMR (400 MHz, DMSO-d₆) δ 9.23 (d, J=5.6 Hz, 1H), 8.75 (d, J=2.2 Hz, 1H), 8.55 (dd, J=4.8, 1.6 Hz, 1H), 8.16 (q, J=5.8, 4.4 Hz, 1H), 7.97 (dt, J=8.0, 2.0 Hz, 1H), 7.49-7.38 (m, 2H), 6.73 (d, J=15.9 Hz, 1H), 4.77 (d, J=5.9 Hz, 1H), 3.18 (q, J=6.5 Hz, 2H), 2.61 (h, J=4.4 Hz, 2H), 2.04 (t, J=7.2 Hz, 2H), 1.60-1.32 (m, 6H), 0.86 (t, J=7.4 Hz, 3H).¹³C NMR (101 MHz, DMSO-d₆) δ 171.22, 164.86, 150.52, 149.53, 135.57, 134.36, 131.23, 124.81, 124.42, 53.52, 38.94, 33.64, 29.16, 23.27, 21.22, 12.04.ESI-MS m/z: 304.91 [M+H]⁺.

### Example 5

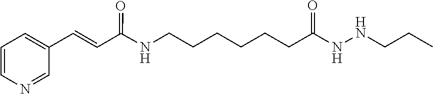 [SMILES: CCCNNC(=O)CCCCCCNC(=O)/C=C/c1cccnc1]

Preparation of (E)-N-(7-oxo-7-(2-propylhydrazino)heptyl)-3-(pyridin-3-yl) acrylamide (LEE3): the synthesis method of LEE1 was used and compounds 11 and 2c were used as raw materials to obtain compound LEE3 as a white solid, with a yield of 57%. ¹H NMR (400 MHz, DMSO-d₆) δ 9.21 (d, J=5.7 Hz, 1H), 8.75 (d, J=2.2 Hz, 1H), 8.55 (dd, J=4.8, 1.6 Hz, 1H), 8.14 (t, J=5.6 Hz, 1H), 7.97 (dt, J=8.0, 2.0 Hz, 1H), 7.49-7.39 (m, 2H), 6.73 (d, J=15.9 Hz, 1H), 4.76 (d, J=6.2 Hz, 1H), 3.17 (q, J=6.6 Hz, 2H), 2.61 (td, J=6.9, 4.5 Hz, 2H), 2.01 (t, J=7.4 Hz, 2H), 1.57-1.34 (m, 6H), 1.28 (tq, J=14.3, 8.6, 8.0 Hz, 4H), 0.86 (t, J=7.4 Hz, 3H). ¹³C NMR (101 MHz, DMSO-d₆) δ 171.36, 164.84, 150.51, 149.53, 135.52, 134.35, 131.25, 124.84, 124.42, 53.50, 39.15, 33.91, 29.46, 28.74, 26.65, 25.63, 21.22, 12.04. ESI-MS m/z: 332.04 [M+H]⁺.

### Example 6

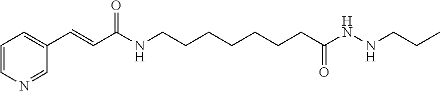 [SMILES: CCCNNC(=O)CCCCCCCNC(=O)/C=C/c1cccnc1]

Preparation of (E)-N-(8-oxo-8-(2-propylhydrazino)octyl)-3-(pyridin-3-yl) acrylamide (LEE4): the synthesis method of LEE1 was used and compounds 11 and 2d were used as raw materials to obtain compound LEE4 as a white solid, with a yield of 60%. ¹H NMR (400 MHz, DMSO-d₆) δ 10.04 (s, 1H), 9.85 (s, 1H), 8.71 (d, J=2.3 Hz, 1H), 8.50 (dd, J=4.7, 1.6 Hz, 1H), 8.11 (t, J=5.6 Hz, 1H), 7.93 (dt, J=8.0, 2.0 Hz, 1H), 7.72 (d, J=8.7 Hz, 2H), 7.62 (s, 2H), 7.45-7.35 (m, 2H), 6.68 (d, J=15.9 Hz, 1H), 3.13 (q, J=6.6 Hz, 2H), 2.69 (t, J=7.1 Hz, 2H), 2.29 (t, J=7.4 Hz, 2H), 1.56 (p, J=6.8 Hz, 2H), 1.42 (h, J=7.3 Hz, 4H), 1.27 (s, 6H), 0.87 (t, J=7.4 Hz, 3H). ¹³C NMR (126 MHz, DMSO-d₆) δ 171.55, 164.84, 150.41, 149.39, 135.50, 134.48, 131.24, 124.81, 124.47, 52.86, 39.14, 33.65, 29.50, 28.87, 28.83, 26.79, 25.38, 20.05, 11.73. ESI-MS m/z: 346.97 [M+H]⁺.

### Example 7

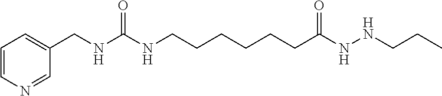 [SMILES: CCCNNC(=O)CCCCCCNC(=O)NCc1cccnc1]

Preparation of 1-(7-oxo-7-(2-propylhydrazino)heptyl)-3-(pyridin-3-ylmethyl) urea (LEE5): the synthesis method of LEE1 was used and compounds 11 and 5 were used as raw materials to obtain compound LEES as a white solid, with a yield of 61%. ¹H NMR (400 MHz, DMSO-d₆) δ 9.19 (s, 1H), 8.41 (d, J=1.9 Hz, 1H), 8.39 (dd, J=4.8, 1.7 Hz, 1H), 7.59 (dt, J=7.9, 1.9 Hz, 1H), 7.29 (dd, J=7.8, 4.8, 0.9 Hz, 1H), 6.32 (t, J=6.1 Hz, 1H), 5.93 (t, J=5.7 Hz, 1H), 4.17 (d, J=5.9 Hz, 2H), 2.93 (q, J=6.6 Hz, 2H), 2.56 (t, J=7.1 Hz, 2H), 1.95 (t, J=7.3 Hz, 2H), 1.43 (t, J=7.2 Hz, 2H), 1.37-1.29 (m, 3H), 1.19 (s, 4H), 0.81 (t, J=7.4 Hz, 3H); ¹³C NMR (126 MHz, DMSO-d₆) δ 171.35, 158.49, 149.01, 148.24, 136.96, 135.29, 123.82, 109.99, 53.44, 41.03, 33.89, 30.33, 28.77, 26.54, 25.65, 21.15, 12.03. ESI-MS m/z: 336.12 [M+H]⁺.

### Example 8

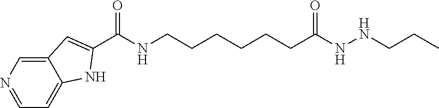 [SMILES: CCCNNC(=O)CCCCCCNC(=O)c1cc2cnccc2[nH]1]

Preparation of N-(7-oxo-7-(2-propylhydrazino) heptyl)-3H-pyrrolo[3,2-c]pyridin-2-carboxamide (LEE7): the synthesis method of LEE1 was used and compounds 11 and 7 were used as raw materials to obtain compound LEE7 as a white solid, with a yield of 56%. ¹H NMR (400 MHz, DMSO-d₆) δ 9.19 (d, J=7.9 Hz, 1H), 8.93 (s, 1H), 8.60 (t, J=5.7 Hz, 1H), 8.21 (d, J=5.8 Hz, 1H), 7.37 (d, J=5.8 Hz, 1H), 7.25 (s, 1H), 3.27 (s, 2H), 2.60 (t, J=7.1 Hz, 2H), 2.01 (t, J=7.3 Hz, 2H), 1.59-1.46 (m, 4H), 1.42-1.23 (m, 7H), 0.86 (t, J=7.4 Hz, 3H). ¹³C NMR (101 MHz, DMSO-d₆) δ 171.36, 160.86, 145.13, 141.64, 139.89, 133.77, 124.88, 107.83, 101.93, 53.49, 33.91, 29.52, 28.77, 26.66, 25.64, 21.22, 12.05. ESI-MS m/z: 346.07 [M+H]⁺.

### Example 9

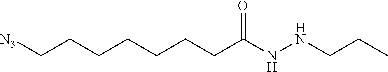 [SMILES: CCCNNC(=O)CCCCCCCN=[N+]=[N-]]

Preparation of 8-azido-N′-propyloctanehydrazide (LEE6): the synthesis method of 1a was used, compounds 8 and 11 were used as raw materials to obtain compound LEE6 as colorless oil, with a yield of 56%. ESI-MS m/z: 241.98 [M+H]⁺.

### Preparation of Compounds in Route 4

### Example 10

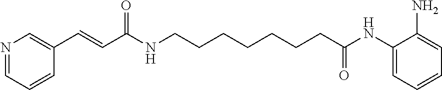 [SMILES: Nc1ccccc1NC(=O)CCCCCCCNC(=O)/C=C/c1cccnc1]

Preparation of (E)-N-(2-aminophenyl)-8-(3-(pyridin-3-yl)acrylamido) octanamide (LEE8): the synthesis method of 1a was used and compound 2d and 1,2-diaminobenzene were used as raw materials to obtain compound LEE8 as a white solid, with a yield of 53%. ¹H NMR (400 MHz, DMSO-d₆) δ 9.05 (s, 1H), 8.71 (s, 1H), 8.51 (d, J=4.8 Hz, 1H), 8.12 (t, J=5.7 Hz, 1H), 7.93 (d, J=7.9 Hz, 1H), 7.47-7.34 (m, 2H), 7.11 (d, J=7.8 Hz, 1H), 6.85 (t, J=7.6 Hz, 1H), 6.74-6.63 (m, 2H), 6.50 (t, J=7.5 Hz, 1H), 4.82 (s, 1H), 3.14 (q, J=6.5 Hz, 2H), 2.27 (t, J=7.4 Hz, 2H), 1.56 (p, J=7.0 Hz, 2H), 1.43 (p, J=7.8 Hz, 2H), 1.28 (s, 7H). ¹³C NMR (126 MHz, DMSO-d₆) δ 171.62, 164.82, 150.48, 149.50, 142.26, 135.51, 134.36, 131.23, 126.13, 125.72, 124.82, 124.42, 124.09, 116.69, 116.38, 39.17, 36.21, 29.55, 29.11, 29.00, 26.86, 25.73. ESI-MS m/z: 381.05 [M+H]⁺.

### Example 11

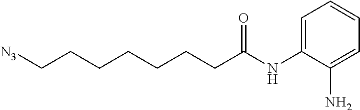 [SMILES: [N-]=[N+]=NCCCCCCCC(=O)Nc1ccccc1N]

Preparation of N-(2-aminophenyl)-8-azidooctanamide (20): the synthesis method of 1a was used and compound 8 and 1,2-diaminobenzene were used as raw materials to obtain compound 20 as a white solid, with a yield of 60%. ¹H NMR (400 MHz, DMSO-d₆) δ 9.05 (s, 1H), 7.10 (d, J=7.9 Hz, 1H), 6.85 (t, J=7.6 Hz, 1H), 6.67 (d, J=8.0 Hz, 1H), 6.50 (d, J=7.5 Hz, 1H), 4.75 (s, 2H), 2.27 (t, J=7.4 Hz, 2H), 1.52 (dt, J=20.1, 7.0 Hz, 4H), 1.28 (s, 8H). ESI-MS m/z: 375.06 [M+H]⁺.

### Preparation of Compounds in Route 5

### Example 12

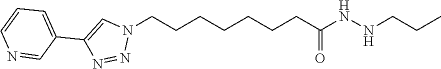 [SMILES: CCCNNC(=O)CCCCCCCn1cc(-c2cccnc2)nn1]

Preparation of n-propyl-8-(4-(pyridin-3-yl)-1H-1,2,3-triazole-1-yl) octanehydrazide (LEE10): compound 19 (60.2 mg, 0.25 mmol) and 3-ethynylpyridine (26 mg, 0.25 mmol) were dissolved in a solution of THF/H2O (v: v=2:1), then sodium ascorbate (50 mg, 0.25 mmol) and copper sulfate (4 mg, 0.025 mmol) were added, and the above materials reacted overnight at room temperature. After the reaction was completed, the reaction solution was washed with water three times and dried over Na2SO4. After filtration, the solvent was completely evaporated to obtain compound LEE10 as a white solid powder (0.07 g, 84%). ¹H NMR (400 MHz, DMSO-d₆) δ 9.19 (s, 1H), 9.01 (d, J=2.2 Hz, 1H), 8.68 (s, 1H), 8.50 (dd, J=4.8, 1.7 Hz, 1H), 8.17 (dt, J=7.9, 2.0 Hz, 1H), 7.44 (dd, J=8.0, 4.8 Hz, 1H), 4.37 (t, J=7.1 Hz, 2H), 2.55 (t, J=7.1 Hz, 1H), 1.95 (t, J=7.3 Hz, 1H), 1.82 (p, J=7.1 Hz, 2H), 1.43 (p, J=7.3 Hz, 2H), 1.35-1.16 (m, 8H), 0.80 (t, J=7.4 Hz, 2H). ¹³C NMR (126 MHz, DMSO-d₆) δ 171.33, 149.26, 146.79, 143.92, 132.79, 127.26, 124.46, 122.39, 53.44, 50.06, 33.84, 30.00, 28.76, 28.50, 26.17, 25.52, 21.16, 12.01. ESI-MS m/z: 345.06 [M+H]⁺.

### Example 13

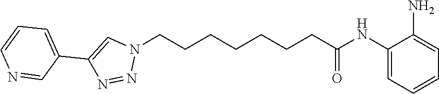 [SMILES: Nc1ccccc1NC(=O)CCCCCCCn1cc(-c2cccnc2)nn1]

Preparation of N-(2-aminophenyl)-8-(4-(pyridin-3-yl)-1H-1,2,3-triazole-1-yl) octanamide (LEE11): the synthesis method of LEE10 was used, compound 20 and 3-ethynylpyridine were used as raw materials to obtain compound LEE11 as a white solid, with a yield of 76%. ¹H NMR (400 MHz, DMSO-d₆) δ 9.08 (s, 1H), 9.01 (d, J=2.2 Hz, 1H), 8.68 (s, 1H), 8.50 (dd, J=4.9, 1.7 Hz, 1H), 8.17 (dt, J=7.9, 2.1 Hz, 1H), 7.44 (dd, J=7.9, 4.8 Hz, 1H), 7.10 (dd, J=7.9, 1.5 Hz, 1H), 6.84 (td, J=7.6, 1.6 Hz, 1H), 6.67 (dd, J=8.0, 1.5 Hz, 1H), 6.49 (td, J=7.6, 1.5 Hz, 1H), 4.90 (s, 1H), 4.39 (t, J=7.1 Hz, 2H), 2.27 (t, J=7.4 Hz, 2H), 1.84 (p, J=7.2 Hz, 2H), 1.54 (p, J=7.3 Hz, 2H), 1.40-1.20 (m, 6H). ¹³C NMR (126 MHz, DMSO-d₆) δ 171.60, 149.24, 146.78, 143.92, 142.20, 132.82, 127.27, 126.11, 125.69, 124.47, 124.11, 122.40, 116.70, 116.39, 50.08, 36.16, 30.03, 28.93, 28.62, 26.21, 25.65. ESI-MS m/z: 379.07 [M+H]⁺.

### Preparation of Compounds in Route 6

### Example 14

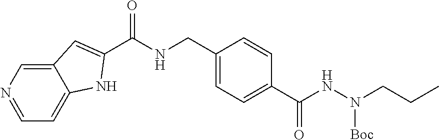 [SMILES: CCCN(NC(=O)c1ccc(CNC(=O)c2cc3cnccc3[nH]2)cc1)C(=O)OC(C)(C)C]

Preparation of tert-butyl 2-(4-((3H-pyrrolo[3,2-c]pyridin-2-carboxamide)methyl) benzoyl)-1-propylhydrazine-1-carboxylate (23c): 1H-pyrrolo[3,2-c]pyridin-2-carboxylic acid (0.48 g, 3 mmol) was dissolved in 15 mL of dichloromethane, and O-benzotriazole-N,N,N′,N′-tetramethylurea tetrafluoroborate (TBTU, 1.05 g, 3.6 mmol) and triethylamine (0.6 mL, 4.5 mmol) were added to the reaction solution in an ice bath. After 30 minutes, compound 15a (1.0 g, 3.3 mmol) was added and then the above materials reacted overnight. The reaction solution was washed with saturated NaHCO₃ (2×30 mL) and brine (2×30 mL) and dried over MgSO₄. After filtration, the solvent was completely evaporated, and then the obtained product was purified by flash chromatography to obtain a white solid powder (0.7 g, 54%). ¹H NMR (400 MHz, DMSO-d₆) δ 10.46 (d, J=5.5 Hz, 1H), 9.29 (t, J=6.1 Hz, 1H), 8.93 (s, 1H), 8.20 (d, J=5.8 Hz, 1H), 7.78 (dd, J=16.8, 7.9 Hz, 2H), 7.41 (d, J=8.1 Hz, 2H), 7.36 (d, J=5.9 Hz, 1H), 7.32 (d, J=4.0 Hz, 1H), 4.55 (d, J=5.9 Hz, 2H), 3.05 (q, J=7.3 Hz, 1H), 1.53-1.42 (m, 2H), 1.39-1.28 (m, 9H), 0.83 (t, J=7.1 Hz, 3H). ESI-MS m/z: 451.88 [M+H]⁺.

Preparation of tert-butyl 2-(4-(((1H-indole-2-carboxyamido) methyl) benzoyl)-1-propylhydrazin-1-carboxylate (23g): the synthesis method of 23c was used, and 2-indolecarboxylic acid and compound 15a were used as raw materials to obtain compound 23g as a white solid, with a yield of 56%. ¹H NMR (400 MHz, DMSO-d₆) δ 11.60 (s, 1H), 10.46 (d, J=7.5 Hz, 1H), 9.09 (t, J=6.0 Hz, 1H), 7.78 (dd, J=16.6, 7.8 Hz, 2H), 7.58 (d, J=8.0 Hz, 1H), 7.40 (dd, J=8.2, 3.6 Hz, 3H), 7.15 (d, J=2.6 Hz, 2H), 7.00 (t, J=7.5 Hz, 1H), 4.53 (d, J=5.9 Hz, 2H), 1.51-1.41 (m, 1H), 1.34 (s, 9H), 0.84 (q, J=7.1, 6.7 Hz, 3H). ESI-MS m/z: 451.05 [M+H]⁺.

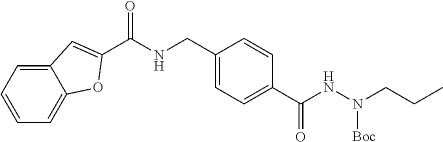 [SMILES: CCCN(NC(=O)c1ccc(CNC(=O)c2cc3ccccc3o2)cc1)C(=O)OC(C)(C)C]

Preparation of tert-butyl 2-(4-((benzofuran-2-carboxyamido)methyl)benzoyl)-1-propylhydrazin-1-carboxylate (23a): the synthesis method of 23c was used, and 2-benzofurancarboxylic acid and compound 15a were used as raw materials to obtain compound 23a as a white solid, with a yield of 56%. ¹H NMR (400 MHz, DMSO-d₆) δ 10.47 (d, J=5.4 Hz, 1H), 9.36 (t, J=6.2 Hz, 1H), 7.82-7.72 (m, 3H), 7.63 (d, J=8.3 Hz, 1H), 7.56 (s, 1H), 7.42 (t, J=7.2 Hz, 3H), 7.30 (t, J=7.5 Hz, 1H), 4.51 (d, J=5.9 Hz, 2H), 1.46 (q, J=7.1 Hz, 2H), 1.39-1.28 (m, 9H), 0.83 (t, J=7.0 Hz, 3H). ESI-MS m/z: 451.08 [M+H]⁺.

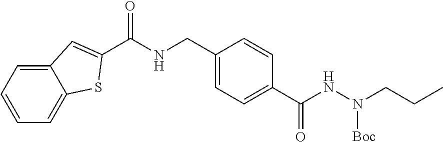 [SMILES: CCCN(NC(=O)c1ccc(CNC(=O)c2cc3ccccc3s2)cc1)C(=O)OC(C)(C)C]

Preparation of tert-butyl 2-(4-((benzothiophene-2-carboxyamido) methyl) benzoyl)-1-propylhydrazin-1-carboxylate (23b): the synthesis method of 23c was used, and benzothiophene-2-carboxylic acid and compound 15a were used as raw materials to obtain compound 23b as a white solid, with a yield of 53%. ¹H NMR (400 MHz, DMSO-d₆) δ 10.47 (d, J=5.9 Hz, 1H), 9.38 (t, J=6.0 Hz, 1H), 8.11 (s, 1H), 8.05-7.95 (m, 1H), 7.96-7.87 (m, 1H), 7.78 (dd, J=16.9, 7.9 Hz, 2H), 7.47-7.37 (m, 5H), 4.51 (d, J=5.8 Hz, 2H), 1.46 (q, J=7.1 Hz, 2H), 1.39-1.29 (m, 6H), 0.83 (t, J=7.3 Hz, 3H). ESI-MS m/z: 467.98 [M+H]⁺.

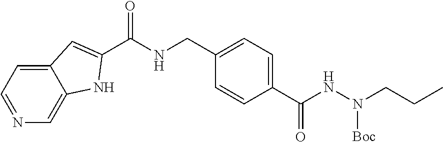 [SMILES: CCCN(NC(=O)c1ccc(CNC(=O)c2cc3ccncc3[nH]2)cc1)C(=O)OC(C)(C)C]

Preparation of tert-butyl 2-(4-((3H-pyrrolo[2,3-c]pyridin-2-carboxyamido)methyl)benzoyl)-1-propylhydrazin-1-carboxylate (23e): the synthesis method of 23c was used, and 1H-pyrrolo[2,3-c]pyridin-2-carboxylic acid and compound 15a were used as raw materials to obtain compound 23e as a white solid, with a yield of 49%. ¹H NMR (400 MHz, DMSO-d₆) δ 12.11 (s, 1H), 10.46 (s, 1H), 9.32 (s, 1H), 8.76 (s, 1H), 8.10 (d, J=5.5 Hz, 1H), 7.85-7.72 (m, 2H), 7.59 (d, J=5.6 Hz, 1H), 7.41 (d, J=8.1 Hz, 2H), 7.19 (s, 1H), 4.55 (d, J=5.9 Hz, 2H), 1.46 (d, J=7.2 Hz, 2H), 1.28 (s, 9H), 0.82 (d, J=7.0 Hz, 3H). ESI-MS m/z: 452.07 [M+H]⁺.

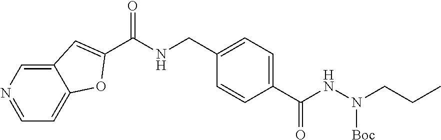 [SMILES: CCCN(NC(=O)c1ccc(CNC(=O)c2cc3cnccc3o2)cc1)C(=O)OC(C)(C)C]

Preparation of tert-butyl 2-(4-(((furan [3,2-c]pyridin-2-carboxyamido) methyl) benzoyl)-1-propylhydroxyzine-1-carboxylate (23d): the synthesis method of 23c was used, and benzo[b]thiophene-2-carboxylic acid and compound 15a were used as raw materials to obtain compound 23d as a white solid, with a yield of 53%. ¹H NMR (400 MHz, DMSO-d₆) δ 10.46 (s, 1H), 9.49 (t, J=6.1 Hz, 1H), 9.05 (s, 1H), 8.55 (d, J=5.8 Hz, 1H), 7.83-7.70 (m, 3H), 7.68 (s, 1H), 7.40 (d, J=8.2 Hz, 2H), 4.51 (d, J=6.0 Hz, 2H), 1.52-1.42 (m, 2H), 1.39-1.28 (m, 9H), 0.83 (t, J=7.2 Hz, 3H). ESI-MS m/z: 453.08 [M+H]⁺.

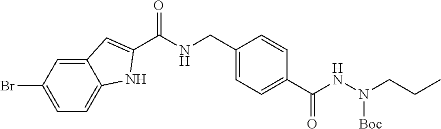 [SMILES: CCCN(NC(=O)c1ccc(CNC(=O)c2cc3cc(Br)ccc3[nH]2)cc1)C(=O)OC(C)(C)C]

Preparation of tert-butyl 2-(4-((5-bromo-1H-indolo-2-carboxyamido)methyl)benzoyl)-1-propylhydrazide-1-carboxylate (23j): the synthesis method of 23c was used, and 5-bromoindole-2-carboxylic acid and compound 15a were used as raw materials to obtain compound 23j as a white solid, with a yield of 58%.

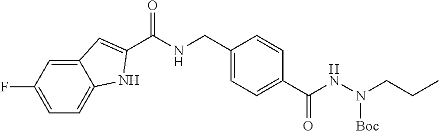 [SMILES: CCCN(NC(=O)c1ccc(CNC(=O)c2cc3cc(F)ccc3[nH]2)cc1)C(=O)OC(C)(C)C]

Preparation of tert-butyl 2-(4-(((5-fluoro-1H-indole-2-carboxyamido)methyl)benzoyl)-1-propylhydrazin-1-carboxylate (23l): the synthesis method of 23c was used, and 5-fluoroindole-2-carboxylic acid and compound 15a were used as raw materials to obtain compound 23l as a white solid, with a yield of 53%.

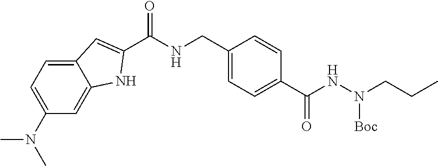 [SMILES: CCCN(NC(=O)c1ccc(CNC(=O)c2cc3ccc(N(C)C)cc3[nH]2)cc1)C(=O)OC(C)(C)C]

Preparation of tert-butyl 2-(4-((6-(dimethylamino)-1H-indole-2-carboxyamido)methyl)benzoyl)-1-propylhydrazin-1-carboxylate (23k): the synthesis method of 23c was used, and 6-(dimethylamino)-1H-indole-2-carboxylic acid and compound 15a were used as raw materials to obtain compound 23k as a white solid, with a yield of 50%.

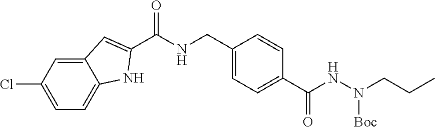 [SMILES: CCCN(NC(=O)c1ccc(CNC(=O)c2cc3cc(Cl)ccc3[nH]2)cc1)C(=O)OC(C)(C)C]

Preparation of tert-butyl 2-(4-((5-chloro-1H-indole-2-carboxyamido)methyl)benzoyl)-1-propylhydrazin-1-carboxylate (23m): the synthesis method of 23c was used, and 5-chloroindole-2-carboxylic acid and compound 15a were used as raw materials to obtain compound 23m as a white solid, with a yield of 51%.

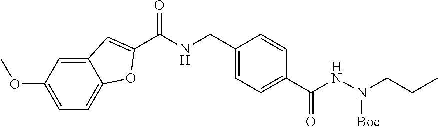 [SMILES: CCCN(NC(=O)c1ccc(CNC(=O)c2cc3cc(OC)ccc3o2)cc1)C(=O)OC(C)(C)C]

Preparation of tert-butyl 2-(4-(((5-methoxybenzofuran-2-carboxyamido)methyl)benzoyl)-1-propylhydrazin-1-carboxylate (23n): the synthesis method of 23c was used, and 5-methoxybenzofuran-2-carboxylic acid and compound 15a were used as raw materials to obtain compound 23n as a white solid, with a yield of 47%.

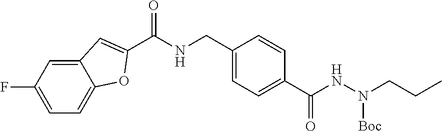 [SMILES: CCCN(NC(=O)c1ccc(CNC(=O)c2cc3cc(F)ccc3o2)cc1)C(=O)OC(C)(C)C]

Preparation of tert-butyl 2-(4-(((5-fluorobenzofuran-2-carboxyamido)methyl)benzoyl)-1-propylhydrazin-1-carboxylate (23o): the synthesis method of 23c was used, and 5-fluorobenzofuran-2-carboxylic acid and compound 15a were used as raw materials to obtain compound 23o as a white solid, with a yield of 59%.

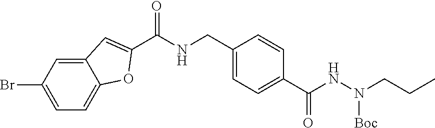 [SMILES: CCCN(NC(=O)c1ccc(CNC(=O)c2cc3cc(Br)ccc3o2)cc1)C(=O)OC(C)(C)C]

Preparation of tert-butyl 2-(4-((5-bromobenzofuran-2-carboxyamido)methyl)benzoyl)-1-propylhydrazin-1-carboxylate (23p): the synthesis method of 23c was used, and 5-bromobenzofuran-2-carboxylic acid and compound 15a were used as raw materials to obtain compound 23p as a white solid, with a yield of 52%.

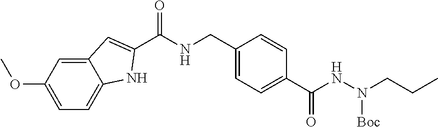 [SMILES: CCCN(NC(=O)c1ccc(CNC(=O)c2cc3cc(OC)ccc3[nH]2)cc1)C(=O)OC(C)(C)C]

Preparation of tert-butyl 2-(4-(((5-methoxy-1H-indole-2-carboxyamido) methyl) benzoyl)-1-propylhydrazin-1-carboxylate (23q): the synthesis method of 23c was used, and 5-methoxyindole-2-carboxylic acid and compound 15a were used as raw materials to obtain compound 23q as a white solid, with a yield of 49%.

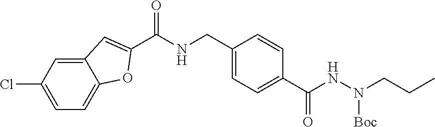 [SMILES: CCCN(NC(=O)c1ccc(CNC(=O)c2cc3cc(Cl)ccc3o2)cc1)C(=O)OC(C)(C)C]

Preparation of tert-butyl 2-(4-((5-chlorobenzofuran-2-carboxyamido)methyl)benzoyl)-1-propylhydrazin-1-carboxylate (23r): the synthesis method of 23c was used, and 5-chlorobenzofuran-2-carboxylic acid and compound 15a were used as raw materials to obtain compound 23r as a white solid, with a yield of 46%.

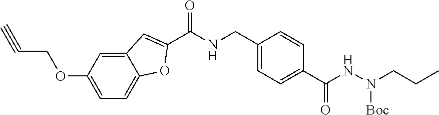 [SMILES: C#CCOc1ccc2oc(C(=O)NCc3ccc(C(=O)NN(CCC)C(=O)OC(C)(C)C)cc3)cc2c1]

Preparation of tert-butyl 2-(4-(((5-(prop-2-yn-1-yloxy)benzofuran-2-carboxyamido) methyl)benzoyl)-1-propylhydrazin-1-carboxylate (23s): the synthesis method of 23c was used, and 5-(prop-2-yn-1-yloxy)benzofuran-2-carboxylic acid and compound 15a were used as raw materials to obtain compound 23s as a white solid, with a yield of 50%.

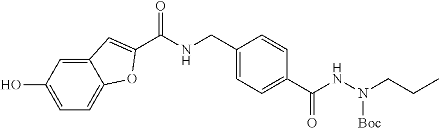 [SMILES: CCCN(NC(=O)c1ccc(CNC(=O)c2cc3cc(O)ccc3o2)cc1)C(=O)OC(C)(C)C]

Preparation of tert-butyl 2-(4-(((5-hydroxybenzofuran-2-carboxyamido)methyl)benzoyl)-1-propylhydrazin-1-carboxylate (23u): the synthesis method of 23c was used, and 5-hydroxybenzofuran-2-carboxylic acid and compound 15a were used as raw materials to obtain compound 23u as a white solid, with a yield of 58%.

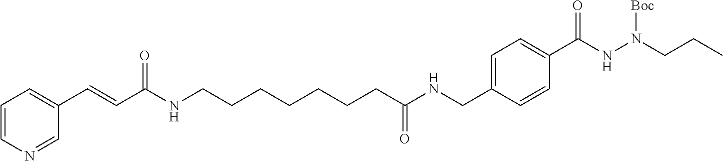 [SMILES: CCCN(NC(=O)c1ccc(CNC(=O)CCCCCCCNC(=O)/C=C/c2cccnc2)cc1)C(=O)OC(C)(C)C]

Preparation of (E)-1-propyl-2-(4-(((8-(3-(pyridin-3-yl)acrylamido)octanamido)methyl)benzoyl)hydrazine-1-carboxylate (23v): compound 2d (0.29 g, 1 mmol) was dissolved in 15 mL of dichloromethane, and 1-(3-dimethylaminopropyl)-3-ethylcarbodiimide hydrochloride (EDCI, 0.23 g, 1.2 mmol) and 1-hydroxybenzotriazole (HOBt at 0° C., 0.18 g, 1.2 mmol) were added in an ice bath. After 30 minutes, compound 15a (0.37 mg, 1.2 mmol) and triethylamine (0.17 mL, 1.2 mmol) were added and the above materials reacted overnight at room temperature. The reaction solution was washed with brine (2×30 mL) and dried over MgSO₄. After filtration, the solvent was completely evaporated, and then the obtained product was purified by flash chromatography to obtain compound 23v as a white solid powder (0.3 g, 51%). ¹H NMR (400 MHz, DMSO-d₆) δ 9.81 (s, 1H), 8.71 (d, J=2.2 Hz, 1H), 8.51 (dd, J=4.8, 1.6 Hz, 1H), 8.11 (t, J=5.7 Hz, 1H), 7.95 (dt, J=8.0, 1.9 Hz, 1H), 7.45-7.36 (m, 2H), 6.68 (d, J=15.9 Hz, 1H), 3.21 (s, 3H), 3.16-3.09 (m, 3H), 2.01 (t, J=7.3 Hz, 2H), 1.56-1.15 (m, 25H), 0.78 (t, J=7.4 Hz, 3H). ESI-MS m/z: 579.96 [M+H]⁺.

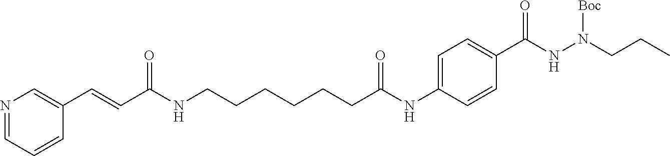 [SMILES: CCCN(NC(=O)c1ccc(NC(=O)CCCCCCNC(=O)/C=C/c2cccnc2)cc1)C(=O)OC(C)(C)C]

Preparation of (E)-1-propyl-2-(4-(7-(3-(pyridin-3-yl)acrylamido)heptanamido)benzoyl) tert-butylhydrazine (carboxylic acid) (23x): the synthesis method of 23v was used, and compounds 2c and 15b were used as raw materials to obtain compound 23x as a white solid, with a yield of 53%. ¹H NMR (400 MHz, DMSO-d₆) δ 10.36 (d, J=3.6 Hz, 1H), 10.12 (s, 1H), 8.72 (d, J=2.2 Hz, 1H), 8.52 (dd, J=4.8, 1.6 Hz, 1H), 8.15 (t, J=5.7 Hz, 1H), 7.96 (dd, J=8.0, 2.0 Hz, 1H), 7.69-7.67 (m, 4H), 7.49 (d, J=1.3 Hz, 1H), 6.69 (d, J=15.9 Hz, 1H), 3.13 (t, J=6.5 Hz, 2H), 2.32-2.26 (m, 2H), 1.45 (d, J=7.2 Hz, 2H), 1.41-1.26 (m, 13H), 1.22 (t, J=6.2 Hz, 4H), 0.83 (t, J=6.1 Hz, 3H). ESI-MS m/z: 552.06 [M+H]⁺.

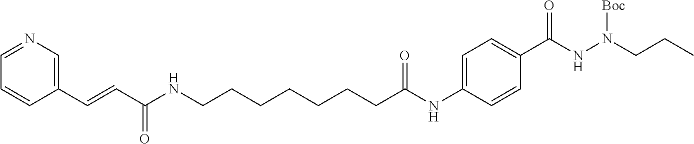 [SMILES: CCCN(NC(=O)c1ccc(NC(=O)CCCCCCCNC(=O)/C=C/c2cccnc2)cc1)C(=O)OC(C)(C)C]

Preparation of (E)-1-propyl-2-(4-(8-(3-(pyridin-3-yl)acrylamido)octanamido)benzoyl)hydrazine tert-butyl-1-carboxylate (23w): the synthesis method of 23v was used, and compounds 2d and 15b were used as raw materials to obtain compound 23w as a white solid, with a yield of 51%. ¹H NMR (400 MHz, DMSO-d₆) δ 10.34 (s, 1H), 10.09 (s, 1H), 8.71 (s, 1H), 8.50 (s, 1H), 8.11 (t, J=5.7 Hz, 1H), 7.93 (d, J=7.9 Hz, 1H), 7.74 (d, J=9.7 Hz, 2H), 7.65 (s, 2H), 7.42-7.37 (m, 3H), 6.68 (d, J=15.9 Hz, 1H), 3.13 (q, J=6.7 Hz, 3H), 2.30 (t, J=7.4 Hz, 2H), 1.56 (t, J=7.1 Hz, 2H), 1.52-1.34 (m, 8H), 1.33-1.16 (m, 13H), 0.84 (t, J=8.4 Hz, 3H). ESI-MS m/z: 566.04 [M+H]⁺.

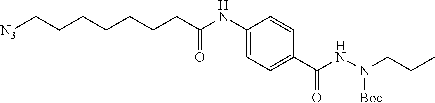 [SMILES: CCCN(NC(=O)c1ccc(NC(=O)CCCCCCCN=[N+]=[N-])cc1)C(=O)OC(C)(C)C]

Preparation of tert-butyl 2-(4-(8-azidooctaamino)benzoyl)-1-propylhydrazin-1-carboxylate (24): the synthesis methods of 23v and LEE37 were used and compound 8 and 13b were used as raw materials to obtain compound 24 as a white solid, with a yield of 50%. ¹H NMR (400 MHz, DMSO-d₆) § 10.34 (s, 1H), 10.08 (s, 1H), 7.74 (d, J=10.2 Hz, 2H), 7.64 (d, J=8.7 Hz, 2H), 2.29 (t, J=7.4 Hz, 2H), 1.55 (t, J=7.0 Hz, 2H), 1.48 (q, J=6.9 Hz, 4H), 1.40 (s, 4H), 1.28 (d, J=3.7 Hz, 13H), 0.84 (t, J=7.2 Hz, 3H). ESI-MS m/z: 461.12 [M+H]⁺.

### Example 15

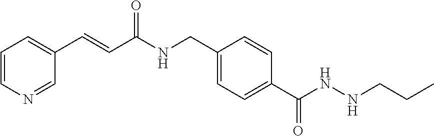 [SMILES: CCCNNC(=O)c1ccc(CNC(=O)/C=C/c2cccnc2)cc1]

Preparation of (E)-N-(4-(2-propylhydrazino-1-carbonyl) benzyl)-3-(pyridin-3-yl) acrylamide (LEE12): the synthesis method of LEE1 was used and compounds 13b and (E)-3-(pyridin-3-yl)acrylic acid were used as raw materials to obtain compound LEE12 as a white solid, with a yield of 52%. ¹H NMR (600 MHz, DMSO-d₆) δ 9.98 (s, 1H), 8.88-8.68 (m, 2H), 8.56 (dd, J=4.8, 1.6 Hz, 1H), 8.00 (dt, J=8.0, 2.0 Hz, 1H), 7.85-7.76 (m, 2H), 7.53 (d, J=15.9 Hz, 1H), 7.50-7.42 (m, 1H), 7.37 (d, J=8.3 Hz, 2H), 6.82 (d, J=15.9 Hz, 1H), 5.11 (s, 1H), 4.46 (d, J=6.0 Hz, 2H), 2.75 (t, J=7.1 Hz, 2H), 1.47 (h, J=7.4 Hz, 2H), 0.91 (t, J=7.5 Hz, 3H). ¹³C NMR (101 MHz, DMSO-d₆) δ 165.57, 165.14, 150.67, 149.64, 143.10, 136.30, 134.46, 132.34, 131.12, 127.61, 124.45, 124.39, 53.58, 42.55, 21.33, 12.13. ESI-MS m/z: 339.08 [M+H]⁺.

### Example 16

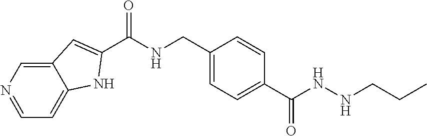 [SMILES: CCCNNC(=O)c1ccc(CNC(=O)c2cc3cnccc3[nH]2)cc1]

Preparation of N-(4-(2-propylhydrazin-1-carbonyl)benzyl)-1H-pyrrolo[3,2-c]pyridin-2-carboxamide (LEE18): compound 23c (0.22 g, 0.5 mmol) was dissolved in 15 mL of dichloromethane, trifluoroacetic acid (TFA, 0.11 g, 1.0 mmol) was added, and the above materials reacted overnight at room temperature. After the reaction was completed, triethylamine (0.1 g, 1.0 mmol) was added to adjust the pH to 8. Then the solution was washed with brine and dried over Na₂SO₄. After filtration, the solvent was completely evaporated, and then the obtained product was purified by flash chromatography to obtain compound LEE18 as a white solid powder (0.14 g, 85%). ¹H NMR (400 MHz, DMSO-d₆) δ 9.42 (t, J=6.1 Hz, 1H), 9.24 (s, 1H), 8.30 (d, J=6.5 Hz, 1H), 7.73-7.68 (m, 2H), 7.67-7.63 (m, 1H), 7.52 (d, J=0.9 Hz, 1H), 7.35-7.28 (m, 2H), 4.49 (d, J=6.0 Hz, 2H), 2.64 (t, J=7.1 Hz, 2H), 1.35 (h, J=7.3 Hz, 2H), 0.79 (t, J=7.4 Hz, 3H). ¹³C NMR (101 MHz, DMSO-d₆) δ 165.56, 160.21, 142.79, 141.87, 140.84, 136.81, 135.09, 132.44, 127.68, 127.57, 124.42, 109.75, 104.54, 53.55, 42.64, 21.28, 12.12. ESI-MS m/z: 351.86 [M+H]⁺.

### Example 17

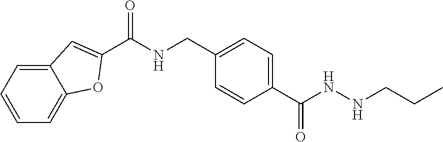 [SMILES: CCCNNC(=O)c1ccc(CNC(=O)c2cc3ccccc3o2)cc1]

Preparation of N-(4-(2-propylhydrazino-1-carbonyl)benzyl)benzofuran-2-carboxyamide (LEE16): the synthesis method of LEE18 was used and compounds 23a and trifluoroacetic acid were used as raw materials to obtain compound LEE16 as a white solid, with a yield of 79%. ¹H NMR (400 MHz, DMSO-d₆) δ 9.90 (d, J=6.0 Hz, 1H), 9.27 (t, J=6.2 Hz, 1H), 7.75-7.68 (m, 3H), 7.59 (dd, J=8.4, 1.1 Hz, 1H), 7.51 (d, J=1.1 Hz, 1H), 7.40 (ddd, J=8.4, 7.2, 1.4 Hz, 1H), 7.33 (d, J=8.1 Hz, 2H), 7.30-7.24 (m, 1H), 5.00 (q, J=6.0 Hz, 1H), 4.47 (t, J=6.3 Hz, 2H), 2.67 (q, J=6.8 Hz, 2H), 1.39 (h, J=7.3 Hz, 2H), 0.83 (t, J=7.4 Hz, 3H). ¹³C NMR (101 MHz, DMSO-d₆) δ 165.60, 158.72, 154.75, 149.49, 143.00, 132.36, 127.64, 127.60, 127.35, 124.20, 123.26, 112.27, 110.12, 53.57, 42.42, 21.32, 12.12. ESI-MS m/z: 352.07 [M+H]⁺.

### Example 18

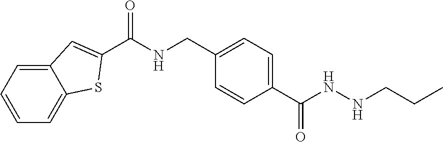 [SMILES: CCCNNC(=O)c1ccc(CNC(=O)c2cc3ccccc3s2)cc1]

Preparation of N-(4-(2-propylhydrazino-1-carbonyl)benzyl)benzo[b]thiophene-2-carboxyamide (LEE17): the synthesis method of LEE18 was used and compounds 23b and trifluoroacetic acid were used as raw materials to obtain compound LEE17 as a white solid, with a yield of 83%. ¹H NMR (400 MHz, DMSO-d₆) δ 9.95-9.87 (m, 1H), 9.30 (t, J=6.0 Hz, 1H), 8.08 (s, 1H), 7.98-7.93 (m, 1H), 7.90-7.85 (m, 1H), 7.76-7.70 (m, 2H), 7.41-7.30 (m, 4H), 5.01 (s, 1H), 4.47 (d, J=5.9 Hz, 2H), 2.73-2.60 (m, 2H), 1.39 (h, J=7.3 Hz, 2H), 0.83 (t, J=7.4 Hz, 3H). ¹³C NMR (101 MHz, DMSO-d₆) δ 165.59, 162.10, 143.03, 140.72, 140.17, 139.64, 132.41, 127.64, 127.60, 126.72, 125.69, 125.46, 125.41, 123.29, 53.58, 42.95, 21.32, 12.13. ESI-MS m/z: 367.96 [M+H]⁺.

### Example 19

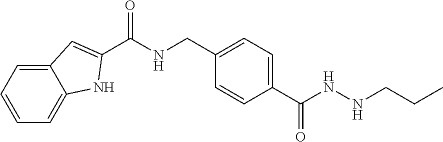 [SMILES: CCCNNC(=O)c1ccc(CNC(=O)c2cc3ccccc3[nH]2)cc1]

Preparation of N-(4-(2-propylhydrazino-1-carbonyl)benzyl)-1H-indole-2-carboxyamide (LEE15): the synthesis method of LEE12 was used and compounds 15a and 1H-indole-2-carboxylic acid were used as raw materials to carry out condensation and deprotection to obtain compound LEE15 as a white solid, with a yield of 56%. ¹H NMR (400 MHz, DMSO-d₆) δ 11.54 (s, 1H), 9.89 (d, J=5.6 Hz, 1H), 9.01 (t, J=6.1 Hz, 1H), 7.75-7.70 (m, 2H), 7.55 (dt, J=7.9, 1.0 Hz, 1H), 7.40-7.30 (m, 3H), 7.15-7.08 (m, 2H), 6.97 (ddd, J=8.0, 6.9, 1.0 Hz, 1H), 5.05-4.94 (m, 1H), 4.48 (d, J=6.0 Hz, 2H), 2.67 (td, J=7.1, 5.7 Hz, 2H), 1.39 (h, J=7.3 Hz, 2H), 0.83 (t, J=7.4 Hz, 3H). ¹³C NMR (101 MHz, DMSO-d₆) δ 165.60, 161.70, 143.45, 136.99, 132.29, 131.98, 127.60, 127.57, 127.44, 123.83, 121.99, 120.22, 112.79, 103.16, 53.58, 42.41, 21.32, 12.13. ESI-MS m/z: 350.86 [M+H]⁺.

### Example 21

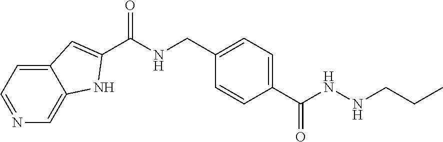 [SMILES: CCCNNC(=O)c1ccc(CNC(=O)c2cc3ccncc3[nH]2)cc1]

Preparation of N-(4-(2-propylhydrazino-1-carbonyl) benzyl)-1H-pyrrolo[2,3-c]pyridine-2-carboxamide (LEE21): the synthesis method of LEE18 was used and compounds 23e and trifluoroacetic acid were used as raw materials to obtain compound LEE21 as a white solid, with a yield of 82%. ¹H NMR (400 MHz, DMSO-d₆) δ 12.06 (s, 1H), 9.90 (s, 1H), 9.25 (t, J=6.1 Hz, 1H), 8.76-8.71 (m, 1H), 8.07 (d, J=5.6 Hz, 1H), 7.78-7.68 (m, 2H), 7.55 (dd, J=5.5, 1.2 Hz, 1H), 7.38-7.30 (m, 2H), 7.15 (s, 1H), 5.04 (s, 1H), 4.51 (d, J=6.0 Hz, 2H), 2.67 (t, J=7.1 Hz, 2H), 1.39 (h, J=7.3 Hz, 2H), 0.84 (t, J=7.4 Hz, 4H). ¹³C NMR (101 MHz, DMSO-d₆) δ 165.57, 161.10, 143.05, 138.53, 136.28, 135.24, 133.77, 132.39, 131.72, 127.64, 127.50, 116.28, 101.96, 53.57, 42.55, 21.32, 12.13. ESI-MS m/z: 352.06 [M+H]⁺.

### Example 22

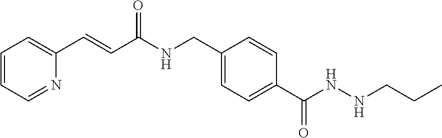 [SMILES: CCCNNC(=O)c1ccc(CNC(=O)/C=C/c2ccccn2)cc1]

Preparation of (E)-N-(4-(2-propylhydrazino-1-carbonyl)benzyl)-3-(pyridin-2-yl) acrylamide (LEE13): the synthesis method of LEE18 was used and compounds 15a and 3-(2-pyridyl) acrylic acid were used as raw materials to obtain compound LEE13 as a white solid, with a yield of 57%. ¹H NMR (400 MHz, DMSO-d₆) δ 9.96 (s, 1H), 8.82 (t, J=6.1 Hz, 1H), 8.58 (d, J=5.2 Hz, 2H), 7.76 (d, J=7.9 Hz, 2H), 7.58-7.25 (m, 6H), 6.88 (d, J=15.9 Hz, 1H), 5.06 (s, 1H), 4.42 (d, J=6.0 Hz, 2H), 2.70 (t, J=7.5 Hz, 2H), 1.42 (h, J=7.1 Hz, 2H), 0.86 (t, J=7.5 Hz, 3H). ¹³C NMR (101 MHz, DMSO-d₆) δ 165.57, 165.31, 165.13, 153.46, 150.67, 150.33, 149.64, 143.13, 143.10, 139.02, 137.66, 136.29, 134.46, 132.34, 131.12, 127.61, 127.53, 125.95, 124.74, 124.53, 124.45, 124.39, 53.58, 42.55, 42.52, 21.32, 12.13. ESI-MS m/z: 339.06 [M+H]⁺.

### Example 23

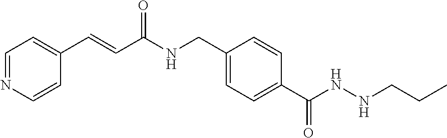 [SMILES: CCCNNC(=O)c1ccc(CNC(=O)/C=C/c2ccncc2)cc1]

Preparation of (E)-N-(4-(2-propylhydrazino-1-carbonyl)benzyl)-3-(pyridin-4-yl)acrylamide (LEE14): the synthesis method of LEE18 was used and compounds 15a and 3-(4-pyridyl) acrylic acid were used as raw materials to obtain compound LEE14 as a white solid, with a yield of 55%. ¹H NMR (400 MHz, DMSO-d₆) δ 9.98 (s, 1H), 8.81-8.69 (m, 2H), 8.56 (dd, J=4.8, 1.6 Hz, 1H), 8.00 (dt, J=8.0, 2.0 Hz, 1H), 7.85-7.76 (m, 2H), 7.53 (d, J=15.9 Hz, 1H), 7.45 (dd, J=8.0, 4.8 Hz, 1H), 7.40-7.32 (m, 2H), 6.82 (d, J=15.9 Hz, 1H), 5.09 (s, 1H), 4.46 (d, J=5.9 Hz, 2H), 2.75 (t, J=7.1 Hz, 2H), 1.47 (h, J=7.3 Hz, 2H), 0.91 (t, J=7.4 Hz, 3H). ¹³C NMR (101 MHz, DMSO-d₆) δ 165.57, 165.14, 150.67, 149.64, 143.10, 136.30, 134.46, 132.34, 131.12, 127.61, 124.45, 124.39, 53.58, 42.55, 21.33, 12.13. ESI-MS m/z: 339.05 [M+H]⁺.

### Example 24

N-(4-(2-propylhydrazin-1-carbonyl)benzyl)thiopheno[3,2-c]pyridin-2-carboxamide (LEE19). The synthesis method of LEE18 was used and compounds 15a and thiopheno[3,2-c]pyridine-2-carboxylic acid were used as raw materials to obtain compound LEE19 as a white solid, with a yield of 54%. ESI-MS m/z: 368.92 [M+H]⁺.

### Example 25

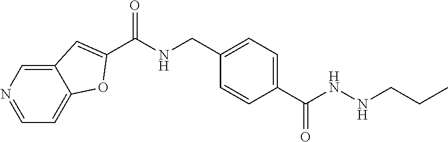 [SMILES: CCCNNC(=O)c1ccc(CNC(=O)c2cc3cnccc3o2)cc1]

Preparation of N-(4-(2-propylhydrazino-1-carbonyl)benzyl)furan[3,2-c]pyridine-2-carboxyamide (LEE20): the synthesis method of LEE18 was used and compounds 23d and trifluoroacetic acid were used as raw materials to obtain compound LEE20 as a white solid, with a yield of 78%. ¹H NMR (400 MHz, DMSO-d₆) δ 9.98 (s, 1H), 9.49 (t, J=6.2 Hz, 1H), 9.05 (s, 1H), 8.55 (d, J=5.8 Hz, 1H), 7.80-7.63 (m, 4H), 7.36 (d, J=7.9 Hz, 2H), 4.49 (d, J=6.1 Hz, 2H), 2.70 (t, J=7.2 Hz, 2H), 1.42 (h, J=7.4 Hz, 2H), 0.86 (t, J=7.4 Hz, 3H); ¹³C NMR (101 MHz, DMSO-d₆) δ 165.64, 158.98, 158.21, 150.06, 146.84, 146.48, 142.86, 132.38, 127.68, 125.05, 108.53, 108.17, 53.58, 42.53, 21.33, 12.19. ESI-MS m/z: 352.88 [M+H]⁺.

### Example 26

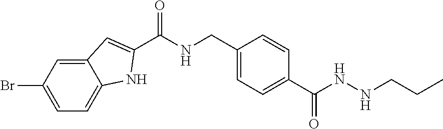 [SMILES: CCCNNC(=O)c1ccc(CNC(=O)c2cc3cc(Br)ccc3[nH]2)cc1]

Preparation of 5-bromo-N-(4-(2-propylhydrazino-1-carbonyl)benzyl)-1H-indole-2-carboxyamide (LEE26): the synthesis method of LEE18 was used and compounds 23j and trifluoroacetic acid were used as raw materials to obtain compound LEE26 as a white solid, with a yield of 84%. ¹H NMR (500 MHz, DMSO-d₆) δ 11.88 (s, 1H), 9.98 (s, 1H), 9.27 (d, J=6.9 Hz, 1H), 7.84 (d, J=1.9 Hz, 1H), 7.81-7.76 (m, 2H), 7.42-7.36 (m, 3H), 7.31-7.26 (m, 1H), 7.18 (s, 1H), 5.06 (s, 1H), 4.53 (d, J=6.0 Hz, 2H), 2.73 (t, J=7.1 Hz, 2H), 1.50-1.39 (m, 2H), 0.89 (t, J=7.4 Hz, 4H).

### Example 27

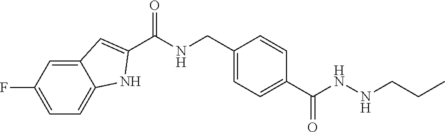 [SMILES: CCCNNC(=O)c1ccc(CNC(=O)c2cc3cc(F)ccc3[nH]2)cc1]

Preparation of 5-fluoro-N-(4-(2-propylhydrazino-1-carbonyl)benzyl)-1H-indole-2-carboxyamide (LEE28): the synthesis method of LEE18 was used and compounds 23l and trifluoroacetic acid were used as raw materials to obtain compound LEE28 as a white solid, with a yield of 85%. ¹H NMR (500 MHz, DMSO-d₆) δ 11.78 (s, 1H), 9.99 (s, 1H), 9.26 (t, J=6.1 Hz, 1H), 7.79 (d, J=8.3 Hz, 2H), 7.45-7.36 (m, 4H), 7.19 (s, 1H), 7.07-6.99 (m, 1H), 5.06 (s, 1H), 4.53 (d, J=6.1 Hz, 2H), 2.73 (t, J=7.1 Hz, 2H), 1.49-1.39 (m, 2H), 0.89 (t, J=7.4 Hz, 4H).

### Example 28

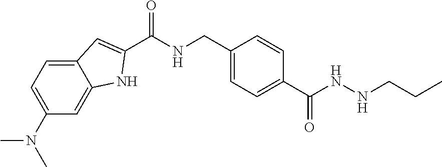 [SMILES: CCCNNC(=O)c1ccc(CNC(=O)c2cc3ccc(N(C)C)cc3[nH]2)cc1]

Preparation of 6-(dimethylamino)-N-(4-(2-propylhydrazino-1-carbonyl)benzyl)-1H-indole-2-carboxyamide (LEE27): the synthesis method of LEE18 was used and compounds 23k and trifluoroacetic acid were used as raw materials to obtain compound LEE27 as a white solid, with a yield of 82%.

### Example 29

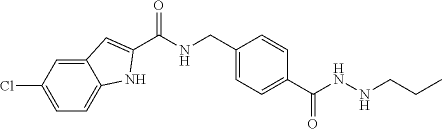 [SMILES: CCCNNC(=O)c1ccc(CNC(=O)c2cc3cc(Cl)ccc3[nH]2)cc1]

Preparation of 5-chloro-N-(4-(2-propylhydrazino-1-carbonyl)benzyl)-1H-indole-2-carboxyamide (LEE29): the synthesis method of LEE18 was used and compounds 23m and trifluoroacetic acid were used as raw materials to obtain compound LEE29 as a white solid, with a yield of 80%. ¹H NMR (500 MHz, DMSO-d₆) δ 11.87 (s, 1H), 9.98 (s, 1H), 9.26 (t, J=6.1 Hz, 1H), 7.79 (d, J=8.3 Hz, 2H), 7.69 (d, J=2.1 Hz, 1H), 7.43 (d, J=8.7 Hz, 1H), 7.39 (d, J=8.3 Hz, 2H), 7.21-7.15 (m, 2H), 5.06 (s, 1H), 4.53 (d, J=6.1 Hz, 2H), 2.73 (t, J=7.1 Hz, 2H), 1.50-1.39 (m, 2H), 0.89 (t, J=7.4 Hz, 4H).

### Example 30

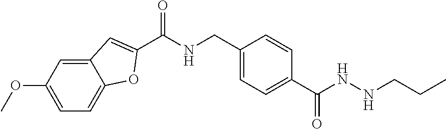 [SMILES: CCCNNC(=O)c1ccc(CNC(=O)c2cc3cc(OC)ccc3o2)cc1]

Preparation of 5-methoxy-N-(4-(2-propylhydrazino-1-carbonyl)benzyl)benzofuran-2-carboxyamide (LEE30): the synthesis method of LEE18 was used and compounds 23n and trifluoroacetic acid were used as raw materials to obtain compound LEE30 as a white solid, with a yield of 85%. ¹H NMR (500 MHz, DMSO-d₆) δ 9.98 (s, 1H), 9.31 (t, J=6.2 Hz, 1H), 7.81-7.75 (m, 2H), 7.57-7.52 (m, 1H), 7.51 (d, J=1.0 Hz, 1H), 7.42-7.34 (m, 2H), 7.26 (d, J=2.6 Hz, 1H), 7.05 (dd, J=9.0, 2.7 Hz, 1H), 4.50 (d, J=6.1 Hz, 2H), 3.79 (s, 3H), 2.73 (t, J=7.1 Hz, 2H), 1.51-1.40 (m, 2H), 0.89 (t, J=7.4 Hz, 3H).

### Example 31

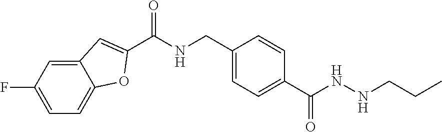 [SMILES: CCCNNC(=O)c1ccc(CNC(=O)c2cc3cc(F)ccc3o2)cc1]

Preparation of 5-fluoro-N-(4-(2-propylhydrazino-1-carbonyl)benzyl)benzofuran-2-carboxyamide (LEE31): the synthesis method of LEE18 was used and compounds 23o and trifluoroacetic acid were used as raw materials to obtain compound LEE31 as a white solid, with a yield of 86%. ¹H NMR (500 MHz, DMSO-d₆) δ 9.96 (d, J=6.2 Hz, 1H), 9.40 (t, J=6.1 Hz, 1H), 7.81-7.74 (m, 2H), 7.72-7.65 (m, 1H), 7.62-7.55 (m, 2H), 7.41-7.36 (m, 2H), 7.35-7.28 (m, 1H), 5.07 (s, 1H), 4.51 (d, J=6.1 Hz, 2H), 2.73 (t, J=7.1 Hz, 2H), 1.45 (h, J=7.3 Hz, 2H), 0.89 (t, J=7.5 Hz, 3H).

### Example 32

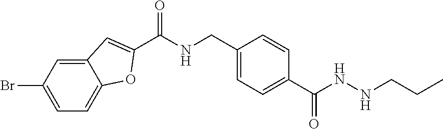 [SMILES: CCCNNC(=O)c1ccc(CNC(=O)c2cc3cc(Br)ccc3o2)cc1]

Preparation of 5-bromo-N-(4-(2-propylhydrazino-1-carbonyl)benzyl)benzofuran-2-carboxyamide (LEE32): the synthesis method of LEE18 was used and compounds 23p and trifluoroacetic acid were used as raw materials to obtain compound LEE32 as a white solid, with a yield of 84%. ¹H NMR (500 MHz, DMSO-d₆) δ 9.98 (s, 1H), 9.45 (t, J=6.2 Hz, 1H), 8.01 (d, J=2.0 Hz, 1H), 7.81-7.75 (m, 2H), 7.64 (d, J=8.8 Hz, 1H), 7.60 (d, J=2.0 Hz, 1H), 7.58 (d, J=0.9 Hz, 2H), 7.39 (d, J=8.2 Hz, 2H), 5.07 (s, 1H), 4.51 (d, J=6.1 Hz, 2H), 2.73 (t, J=7.1 Hz, 2H), 1.53-1.35 (m, 2H), 0.89 (t, J=7.4 Hz, 3H).

### Example 33

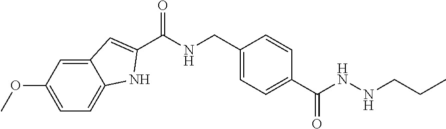 [SMILES: CCCNNC(=O)c1ccc(CNC(=O)c2cc3cc(OC)ccc3[nH]2)cc1]

Preparation of 5-methoxy-N-(4-(2-propylhydrazino-1-carbonyl)benzyl)-1H-indole-2-carboxyamide (LEE33): the synthesis method of LEE18 was used and compounds 23q and trifluoroacetic acid were used as raw materials to obtain compound LEE33 as a white solid, with a yield of 82%. ¹H NMR (500 MHz, DMSO-d₆) δ 11.47 (s, 1H), 9.98 (s, 1H), 9.07 (t, J=6.1 Hz, 1H), 7.81-7.76 (m, 2H), 7.42-7.36 (m, 2H), 7.31 (d, J=8.9 Hz, 1H), 7.12-7.05 (m, 2H), 6.83 (dd, J=8.9, 2.5 Hz, 1H), 5.09 (s, 1H), 4.53 (d, J=6.1 Hz, 2H), 3.75 (s, 3H), 2.73 (t, J=7.1 Hz, 2H), 1.50-1.40 (m, 2H), 0.89 (t, J=7.4 Hz, 3H).

### Example 34

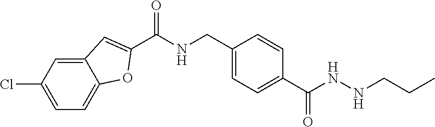 [SMILES: CCCNNC(=O)c1ccc(CNC(=O)c2cc3cc(Cl)ccc3o2)cc1]

Preparation of 5-chloro-N-(4-(2-propylhydrazino-1-carbonyl)benzyl)benzofuran-2-carboxyamide (LEE34): the synthesis method of LEE18 was used and compounds 23r and trifluoroacetic acid were used as raw materials to obtain compound LEE34 as a white solid, with a yield of 86%. ¹H NMR (500 MHz, DMSO-d₆) δ 9.99 (d, J=4.1 Hz, 1H), 9.49 (t, J=6.1 Hz, 1H), 7.87 (d, J=2.2 Hz, 1H), 7.81-7.75 (m, 2H), 7.69 (d, J=8.8 Hz, 1H), 7.60 (d, J=1.0 Hz, 1H), 7.48 (dd, J=8.8, 2.2 Hz, 1H), 7.42-7.36 (m, 2H), 5.09 (s, 1H), 4.51 (d, J=6.1 Hz, 2H), 2.73 (t, J=7.1 Hz, 2H), 1.49-1.39 (m, 3H), 0.89 (t, J=7.4 Hz, 4H).

### Example 35

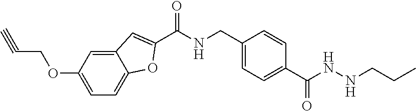 [SMILES: C#CCOc1ccc2oc(C(=O)NCc3ccc(C(=O)NNCCC)cc3)cc2c1]

Preparation of 5-(prop-2-yn-1-yloxy)-N-(4-(2-propylhydrazino-1-carbonyl)benzyl)benzofuran-2-carboxyamide (LEE35): the synthesis method of LEE18 was used and compounds 23s and trifluoroacetic acid were used as raw materials to obtain compound LEE35 as a white solid, with a yield of 83%. ¹H NMR (500 MHz, DMSO-d₆) δ 9.97 (s, 1H), 9.31 (t, J=6.2 Hz, 1H), 7.81-7.74 (m, 2H), 7.61-7.55 (m, 1H), 7.52 (d, J=0.9 Hz, 1H), 7.43-7.36 (m, 2H), 7.34 (d, J=2.6 Hz, 1H), 7.13-7.07 (m, 1H), 5.08 (s, 1H), 4.83 (d, J=2.4 Hz, 2H), 4.51 (d, J=6.1 Hz, 2H), 3.56 (t, J=2.4 Hz, 1H), 2.73 (t, J=7.1 Hz, 2H), 1.45 (h, J=7.4 Hz, 2H), 0.89 (t, J=7.4 Hz, 4H).

### Example 36

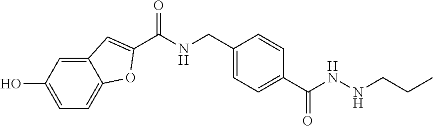 [SMILES: CCCNNC(=O)c1ccc(CNC(=O)c2cc3cc(O)ccc3o2)cc1]

Preparation of 5-hydroxy-N-(4-(2-propylhydrazino-1-carbonyl)benzyl)benzofuran-2-carboxyamide (LEE36): the synthesis method of LEE18 was used and compounds 23u and trifluoroacetic acid were used as raw materials to obtain compound LEE36 as a white solid, with a yield of 79%. ¹H NMR (500 MHz, DMSO-d₆) δ 9.96 (s, 1H), 9.39 (s, 1H), 9.24 (t, J=6.2 Hz, 1H), 7.81-7.74 (m, 2H), 7.46-7.35 (m, 4H), 7.02 (d, J=2.5 Hz, 1H), 6.90 (dd, J=8.9, 2.5 Hz, 1H), 4.49 (d, J=6.2 Hz, 2H), 2.73 (t, J=7.1 Hz, 2H), 1.50-1.39 (m, 2H), 0.89 (t, J=7.4 Hz, 3H).

### Example 37

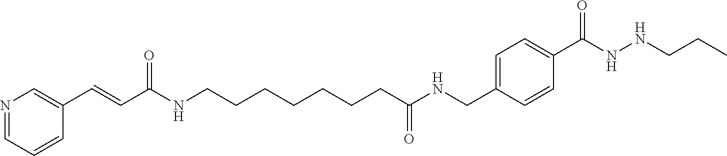 [SMILES: CCCNNC(=O)c1ccc(CNC(=O)CCCCCCCNC(=O)/C=C/c2cccnc2)cc1]

Preparation of (E)-N-(4-(2-propylhydrazino-1-carbonyl)benzyl)-8-(3-(pyridine-3-yl)acrylamido) octanamide (LEE37): the synthesis method of LEE18 was used and compounds 23v and trifluoroacetic acid were used as raw materials to obtain compound LEE37 as a white solid, with a yield of 78%. ¹H NMR (400 MHz, DMSO-d₆) δ 9.93 (s, 1H), 8.71 (s, 1H), 8.50 (d, J=4.7 Hz, 1H), 8.31 (t, J=6.0 Hz, 1H), 8.12 (t, J=5.7 Hz, 1H), 7.93 (dt, J=8.0, 2.0 Hz, 1H), 7.73 (d, J=8.0 Hz, 2H), 7.45-7.36 (m, 2H), 7.25 (d, J=8.0 Hz, 2H), 6.69 (d, J=15.9 Hz, 1H), 5.06 (s, 1H), 4.25 (d, J=5.9 Hz, 2H), 3.13 (q, J=6.6 Hz, 2H), 2.70 (t, J=7.1 Hz, 2H), 2.10 (t, J=7.4 Hz, 2H), 1.55-1.35 (m, 6H), 1.31-1.15 (m, 7H), 0.86 (t, J=7.4 Hz, 3H). ¹³C NMR (126 MHz, DMSO-d₆) δ 172.69, 165.56, 164.83, 150.49, 149.51, 143.60, 135.52, 134.34, 132.12, 131.22, 127.49, 127.36, 124.81, 124.41, 53.55, 42.16, 39.17, 35.76, 29.53, 29.09, 28.94, 26.84, 25.70, 21.30, 12.12. ESI-MS m/z: 479.96 [M+H]⁺.

### Example 38

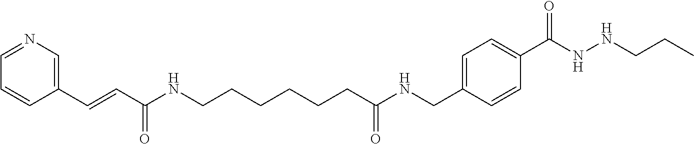 [SMILES: CCCNNC(=O)c1ccc(CNC(=O)CCCCCCNC(=O)/C=C/c2cccnc2)cc1]

Preparation of (E)-N-(4-(2-propylhydrazino-1-carbonyl)benzyl)-7-(3-(pyridine-3-yl)acrylamido) heptanamide (LEE39): the synthesis method of LL289 was used and compounds 15a and 2d were used as raw materials to obtain compound YKR-31 as a white solid, with a yield of 58%. ¹H NMR (400 MHz, DMSO-d₆) δ 9.93 (s, 1H), 8.51 (dd, J=4.7, 1.6 Hz, 1H), 8.32 (t, J=6.1 Hz, 1H), 8.13 (t, J=5.6 Hz, 1H), 7.73 (d, J=8.0 Hz, 2H), 7.46-7.36 (m, 2H), 7.26 (d, J=8.0 Hz, 2H), 6.69 (d, J=15.9 Hz, 1H), 5.03 (s, 1H), 4.25 (d, J=5.8 Hz, 2H), 3.13 (q, J=6.6 Hz, 2H), 7.97-7.90 (m, 1H), 2.70 (t, J=7.0 Hz, 2H), 2.11 (t, J=7.4 Hz, 2H), 1.53-1.38 (m, 6H), 1.24 (dq, J=9.4, 5.7, 5.0 Hz, 4H), 0.86 (t, J=7.4 Hz, 3H), 8.75-8.67 (m, 1H). ¹³C NMR (126 MHz, DMSO-d₆) δ 172.67, 165.57, 164.84, 153.46, 150.50, 149.51, 143.60, 135.53, 135.52, 134.34, 132.12, 131.22, 127.98, 127.50, 127.42, 127.35, 124.81, 124.41, 53.56, 42.19, 42.16, 39.15, 35.75, 29.45, 28.86, 26.69, 25.89, 25.70, 21.30, 12.12, 11.10. ESI-MS m/z: 465.89 [M+H]⁺.

### Example 39

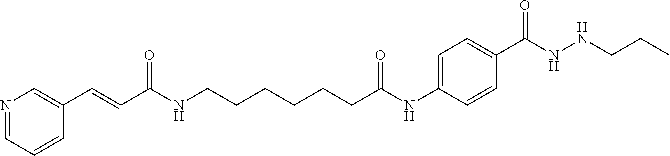 [SMILES: CCCNNC(=O)c1ccc(NC(=O)CCCCCCNC(=O)/C=C/c2cccnc2)cc1]

Preparation of (E)-N-(4-(2-propylhydrazino-1-carbonyl)phenyl)-7-(3-(pyridine-3-yl)acrylamido) heptamide (LEE40): the synthesis method of LEE18 was used and compounds 23x and trifluoroacetic acid were used as raw materials to obtain compound LEE40 as a white solid, with a yield of 65%. ¹H NMR (400 MHz, DMSO-d₆) δ 10.06 (s, 1H), 9.84 (d, J=5.5 Hz, 1H), 8.71 (d, J=2.2 Hz, 1H), 8.50 (dd, J=4.8, 1.6 Hz, 1H), 8.13 (t, J=5.7 Hz, 1H), 7.93 (dt, J=8.1, 2.0 Hz, 1H), 7.72 (d, J=8.6 Hz, 2H), 7.61 (d, J=8.6 Hz, 2H), 7.45-7.35 (m, 2H), 6.69 (d, J=15.9 Hz, 1H), 5.00 (d, J=5.5 Hz, 1H), 3.14 (q, J=6.5 Hz, 2H), 2.69 (q, J=6.4 Hz, 2H), 2.29 (t, J=7.4 Hz, 2H), 1.57 (d, J=7.3 Hz, 2H), 1.43 (p, J=7.3 Hz, 4H), 1.31-1.26 (m, 4H), 0.87 (t, J=7.4 Hz, 3H). ¹³C NMR (126 MHz, DMSO-d₆) δ 172.09, 165.33, 164.83, 150.50, 149.51, 142.38, 135.51, 134.34, 131.22, 128.82, 128.28, 127.85, 124.81, 124.41, 118.63, 53.62, 39.14, 36.87, 29.42, 28.84, 26.71, 25.41, 21.31, 12.12, 11.14. ESI-MS m/z: 451.97 [M+H]⁺.

### Example 40

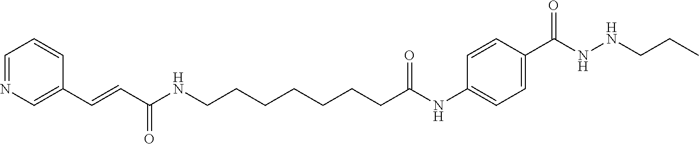 [SMILES: CCCNNC(=O)c1ccc(NC(=O)CCCCCCCNC(=O)/C=C/c2cccnc2)cc1]

Preparation of (E)-N-(4-(2-propylhydrazino-1-carbonyl)phenyl)-8-(3-(pyridine-3-yl)acrylamido) amide (LEE38): the synthesis method of LEE18 was used and compounds 23w and trifluoroacetic acid were used as raw materials to obtain compound LEE38 as a white solid, with a yield of 67%. ¹H NMR (400 MHz, DMSO-d₆) δ 10.04 (s, 1H), 9.85 (s, 1H), 8.71 (d, J=2.3 Hz, 1H), 8.50 (dd, J=4.7, 1.6 Hz, 1H), 8.11 (t, J=5.6 Hz, 1H), 7.93 (dt, J=8.0, 2.0 Hz, 1H), 7.72 (d, J=8.7 Hz, 2H), 7.62 (s, 2H), 7.45-7.35 (m, 2H), 6.68 (d, J=15.9 Hz, 1H), 3.13 (q, J=6.6 Hz, 2H), 2.69 (t, J=7.1 Hz, 2H), 2.29 (t, J=7.4 Hz, 2H), 1.56 (p, J=6.8 Hz, 2H), 1.42 (h, J=7.3 Hz, 4H), 1.27 (s, 6H), 0.87 (t, J=7.4 Hz, 3H). ¹³C NMR (126 MHz, DMSO-d₆) δ 172.10, 165.34, 164.82, 150.49, 149.50, 142.39, 135.51, 134.34, 131.22, 128.29, 127.83, 124.81, 124.41, 118.62, 53.60, 39.15, 36.90, 29.53, 29.08, 28.98, 26.82, 25.42, 21.29, 12.12. ESI-MS m/z: 465.97 [M+H]⁺.

### Example 41

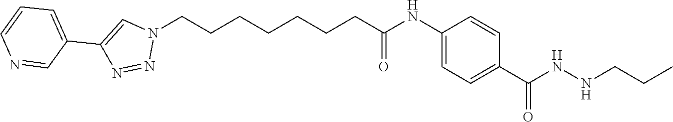 [SMILES: CCCNNC(=O)c1ccc(NC(=O)CCCCCCCn2cc(-c3cccnc3)nn2)cc1]

Preparation of N-(4-(2-propylhydrazino-1-carbonyl)phenyl)-8-(4-(pyridin-3-yl)-1H-1,2,3-triazole-1-yl) octanamide (LEE41): the synthesis method of LEE18 was used, compound 24 and trifluoroacetic acid were used as raw materials to obtain a white solid, with a yield of 67%.

The synthesis method of LEE11 was used, the product from the previous step and 3-ethynylpyridine were used as raw materials to obtain compound LEE41 as a white solid, with a yield of 80%. ¹H NMR (400 MHz, DMSO-d₆) δ 10.08 (s, 1H), 9.93 (s, 1H), 9.01 (d, J=2.2 Hz, 1H), 8.68 (s, 1H), 8.49 (d, J=4.7 Hz, 1H), 8.16 (dd, J=8.0, 2.0 Hz, 1H), 7.72 (d, J=8.5 Hz, 2H), 7.61 (d, J=8.5 Hz, 2H), 7.44 (dd, J=8.0, 4.8 Hz, 1H), 4.38 (t, J=7.1 Hz, 2H), 2.71 (t, J=7.1 Hz, 2H), 2.28 (t, J=7.3 Hz, 2H), 1.83 (p, J=7.2 Hz, 2H), 1.54 (p, J=7.3 Hz, 2H), 1.43 (h, J=7.3 Hz, 2H), 1.35-1.16 (m, 8H), 0.87 (t, J=7.4 Hz, 3H). ¹³C NMR (126 MHz, DMSO-d₆) δ 172.10, 165.34, 149.24, 146.77, 143.92, 142.46, 132.82, 128.33, 127.67, 127.27, 124.47, 122.41, 118.62, 53.53, 50.07, 36.84, 30.01, 28.91, 28.61, 26.17, 25.34, 21.15, 12.08. ESI-MS m/z: 464.03 [M+H]⁺.

### Example 42

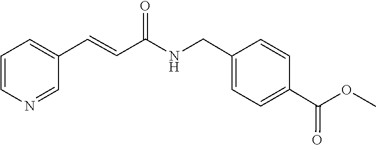 [SMILES: COC(=O)c1ccc(CNC(=O)/C=C/c2cccnc2)cc1]

Preparation of methyl (E)-4-((3-(pyridine-3-yl)acrylamido)methyl) benzoate (LEE43): the synthesis method of 1a was used to obtain compound LEE43 as a white solid, with a yield of 51%. ¹H NMR (400 MHz, DMSO-d₆) δ 8.80-8.78 (m, 2H), 8.57 (dd, J=1.1 Hz, J=3.1 Hz, 1H), 8.03 (td, J=1.1 Hz, J=5.4 Hz, 1H), 7.95 (td, J=1.3 Hz, J=5.5 Hz, 2H), 7.55 (d, J=10.6 Hz, 1H), 7.47-7.43 (m, 3H), 6.84 (d, J=10.6 Hz, 1H), 4.51 (d, J=4.0 Hz, 2H), 3.85 (s, 3H).

### Example 43

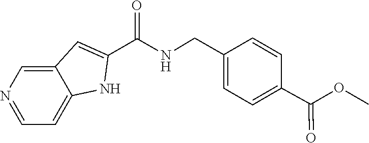 [SMILES: COC(=O)c1ccc(CNC(=O)c2cc3cnccc3[nH]2)cc1]

Preparation of methyl 4-((1H-pyrrolo[3,2-c]pyridine-2-carbonylamide)methyl) benzoate (LEE44): the synthesis method of 1a was used to obtain compound LEE44 as a white solid, with a yield of 46%. ¹H NMR (400 MHz, DMSO-d₆) δ 12.94 (s, 1H), 9.65 (t, J=4.0 Hz, 1H), 9.31 (s, 1H), 8.39 (d, J=4.3 Hz, 1H), 7.96 (d, J=5.6 Hz, 2H), 7.74 (d, J=4.2 Hz, 1H), 7.64 (s, 1H), 7.51 (d, J=5.5 Hz, 2H), 4.64 (d, J=4.0 Hz, 2H), 3.85 (s, 3H).

### Example 44: Inhibitory Activity of Compounds on HDAC1, 2 and 3

Experimental materials: HDAC buffer: 15 mM Tris-HCl (pH 8.0), 250 μM EDTA, 250 mM NaCl, 10% glycerin. Trypsin termination solution: 10 mg/ml pancreatin, 50 mM Tris-HCL (pH 8.0), 100 mM NaCl, 2 μM TSA. Substrates: the dedicated substrates HDAC1, HDAC2 and HDAC3 were dissolved with dimethyl sulfoxide to be formulated into a 30 mM stock solution, the stock solution was diluted to 300 μM with HDAC buffer, so that the content of dimethyl sulfoxide was around 1%. Enzyme solution: HDAC1, HDAC2 and HDAC3 were diluted with HDAC buffer in a ratio of 1:20.

### Experimental Steps:

a) Formulation of 100% solution: 50 μL of HDAC buffer was mixed with 10 μL of enzyme solution, 40 μL of substrate was added after 5 minutes and the above materials reacted at 37° C. for 30 minutes, then 100 μL of trypsin termination solution was added to terminate the above reaction, and the reaction was carried out at 37° C. for 20 minutes, the fluorescence intensity was measured at 390 nm/460 nm to obtain 100% absorbence. AMC was used as the standard to create a standard curve and calculate enzyme activity.

b) Formulation of blank solution: 60 μL of HDAC buffer was added with 40 μL of substrate, and the above materials reacted at 37° C. for 30 minutes, then 100 μL of trypsin termination solution was added and the reaction was carried out at 37° C. for 20 minutes, the fluorescence intensity was measured at 390 nm/460 nm to obtain blank absorbence.

6. The determination steps for drug inhibition of HDAC enzyme activity: 50 μL of HDAC buffer containing a drug was mixed with 10 μL of enzyme solution and pre-incubated for 5 minutes, 40 μL of substrate was added and then the above materials reacted at 37° C. for 30 minutes, then 100 μL of trypsin termination solution was added to terminate the above reaction, and the reaction was carried out at 37° C. for 20 minutes, and the fluorescence intensity was measured at 390 nm/460 nm.

$$Inhibitory rate ( \% ) = \frac{\begin{matrix}100 \% fluorescence intensity - \\ fluorescence intensity of compound\end{matrix}}{100 \% fluorescence intensity - fluorescence intensity of blank} \times 100 \%$$

Finally, the inhibitory rate (%) of the compound was subjected to S-curve fitting with its corresponding concentration to calculate the IC₅₀ value.

The inhibitory activity results of some compounds shown in structural general formulas (I, II, III or IV) of the present disclosure on HDAC1, HDAC2 and HDAC3 are shown in Table 1 below:

TABLE 1
Inhibitory IC₅₀ values of some compounds on HDAC1, 2 and 3

| | | IC₅₀ (nMª) | | |
|---|---|---|---|---|
| | Structure | HDAC1 | HDAC2 | HDAC3 |
| LEE12 | 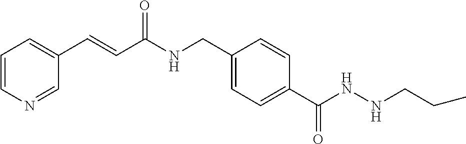 [SMILES: CCCNNC(=O)c1ccc(CNC(=O)/C=C/c2cccnc2)cc1] | 25.10 | 140.8 | 3.12 |
| LEE13 | 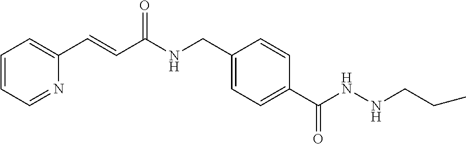 [SMILES: CCCNNC(=O)c1ccc(CNC(=O)/C=C/c2ccccn2)cc1] | 13.20 | 49.17 | 1.82 |
| LEE14 | 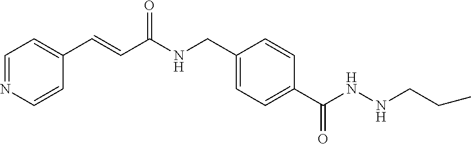 [SMILES: CCCNNC(=O)c1ccc(CNC(=O)/C=C/c2ccncc2)cc1] | 12.71 | 88.48 | 2.74 |
| LEE1 | 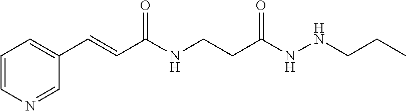 [SMILES: CCCNNC(=O)CCNC(=O)/C=C/c1cccnc1] | >10,000 | 13600 | 1556 |
| LEE2 | 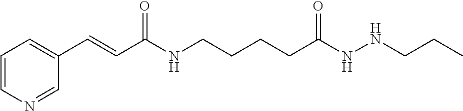 [SMILES: CCCNNC(=O)CCCCNC(=O)/C=C/c1cccnc1] | 689 | 2002 | 111.5 |
| LEE3 | 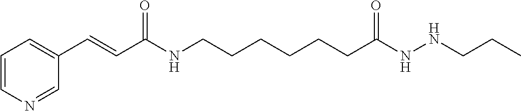 [SMILES: CCCNNC(=O)CCCCCCNC(=O)/C=C/c1cccnc1] | 79.3 | 338.7 | 20.13 |
| LEE18 | 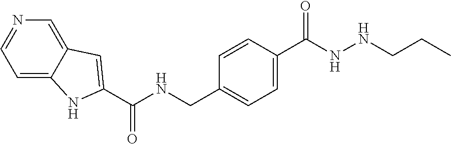 [SMILES: CCCNNC(=O)c1ccc(CNC(=O)c2cc3cnccc3[nH]2)cc1] | 7.61 | 37.81 | 0.71 |
| LEE20 | 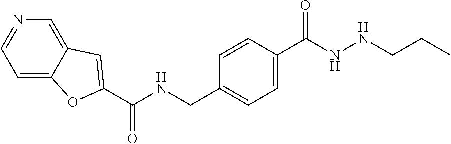 [SMILES: CCCNNC(=O)c1ccc(CNC(=O)c2cc3cnccc3o2)cc1] | 10.35 | 38.41 | 0.52 |
| LEE16 | 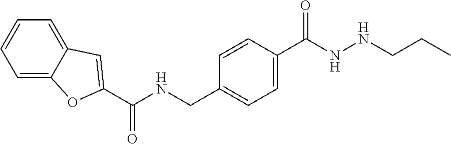 [SMILES: CCCNNC(=O)c1ccc(CNC(=O)c2cc3ccccc3o2)cc1] | 3.01 | 18.54 | 0.435 |
| LEE15 | 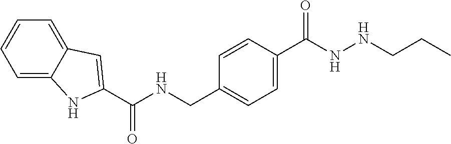 [SMILES: CCCNNC(=O)c1ccc(CNC(=O)c2cc3ccccc3[nH]2)cc1] | 3.52 | 15.14 | 0.383 |
| LEE17 | 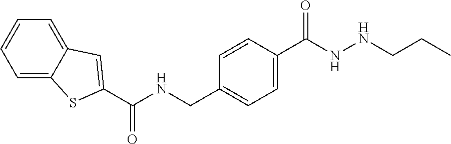 [SMILES: CCCNNC(=O)c1ccc(CNC(=O)c2cc3ccccc3s2)cc1] | 3.79 | 16.92 | 0.551 |
| LEE19 | 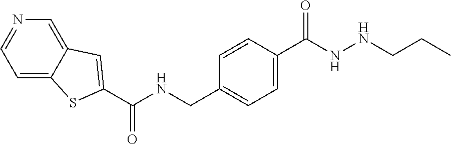 [SMILES: CCCNNC(=O)c1ccc(CNC(=O)c2cc3cnccc3s2)cc1] | 10.06 | 59.96 | 1.20 |
| LEE7 | 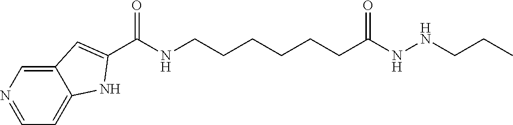 [SMILES: CCCNNC(=O)CCCCCCNC(=O)c1cc2cnccc2[nH]1] | 25.09 | 112.5 | 2.97 |
| LEE21 | 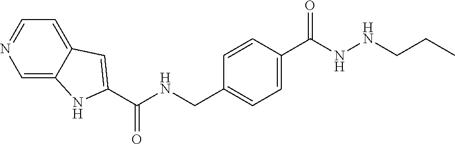 [SMILES: CCCNNC(=O)c1ccc(CNC(=O)c2cc3ccncc3[nH]2)cc1] | 8.17 | 51.47 | 2.42 |
| LEE5 | 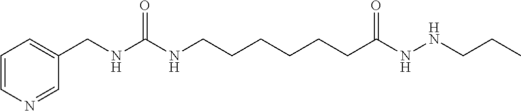 [SMILES: CCCNNC(=O)CCCCCCNC(=O)NCc1cccnc1] | 444 | 398 | 110.4 |
| LEE37 | 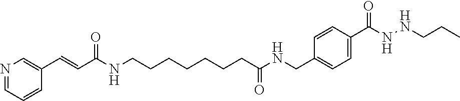 [SMILES: CCCNNC(=O)c1ccc(CNC(=O)CCCCCCCNC(=O)/C=C/c2cccnc2)cc1] | 17.8 | 84.1 | 1.98 |
| LEE8 | 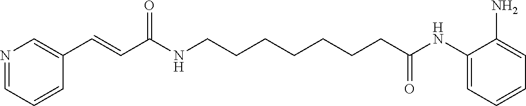 [SMILES: Nc1ccccc1NC(=O)CCCCCCCNC(=O)/C=C/c1cccnc1] | 147.4 | 320.9 | 26.42 |
| LEE39 | 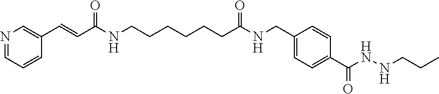 [SMILES: CCCNNC(=O)c1ccc(CNC(=O)CCCCCCNC(=O)/C=C/c2cccnc2)cc1] | 500.8 | 1176 | 84.31 |
| LEE40 | 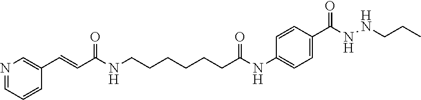 [SMILES: CCCNNC(=O)c1ccc(NC(=O)CCCCCCNC(=O)/C=C/c2cccnc2)cc1] | 53.87 | 122.7 | 12.37 |
| LEE38 | 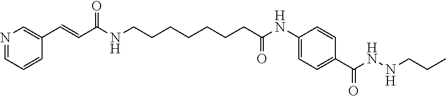 [SMILES: CCCNNC(=O)c1ccc(NC(=O)CCCCCCCNC(=O)/C=C/c2cccnc2)cc1] | 620.1 | 539.1 | 97.37 |
| LEE4 | 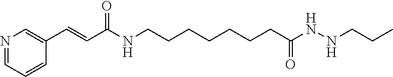 [SMILES: CCCNNC(=O)CCCCCCCNC(=O)/C=C/c1cccnc1] | 241.0 | 505.8 | 76.68 |
| LEE11 | 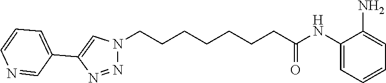 [SMILES: Nc1ccccc1NC(=O)CCCCCCCn1cc(-c2cccnc2)nn1] | 56.06 | 187.7 | 8.78 |
| LEE41 | 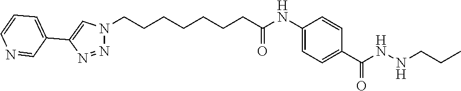 [SMILES: CCCNNC(=O)c1ccc(NC(=O)CCCCCCCn2cc(-c3cccnc3)nn2)cc1] | 18.02 | 78.67 | 1.08 |
| LEE10 | 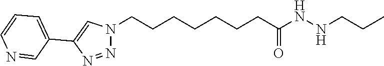 [SMILES: CCCNNC(=O)CCCCCCCn1cc(-c2cccnc2)nn1] | 57.37 | 150.0 | 5.43 |
| Comparative example 1 LP411/3b | 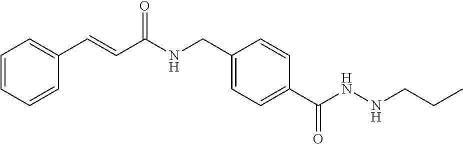 [SMILES: CCCNNC(=O)c1ccc(CNC(=O)/C=C/c2ccccc2)cc1] | 11.8 | 95.45 | 0.95 |
| Comparative example 2 LP97/13a | 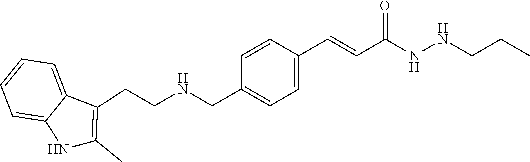 [SMILES: CCCNNC(=O)/C=C/c1ccc(CNCCc2c(C)[nH]c3ccccc23)cc1] | 5.17 | 49.5 | 0.28 |
| SAHA | | 43.76 | 89.23 | 149.7 |
| MS275 | | 53.89 | 108.2 | 77.18 |

ªThe data in the table are all from three independent experiments, and the values are mean values with a standard deviation of <10%;
ND: not detected.

The experimental results show that the vast majority of compounds in the table have nanomolar level of inhibitory ICso values against HDAC1/2/3, and the activity against HDAC1 and HDAC3 is generally higher than that on HDAC2. The activity is generally higher than that of positive control drugs SAHA and MS275.

### Example 45: Inhibitory Activity of cCmpounds on NAMPT

TABLE 2
Inhibitory IC₅₀ values of some compounds on NAMPT

| | | NAMPT IC⁵⁰ |
|---|---|---|
| | Structure | (nMª) |
| LEE12 | 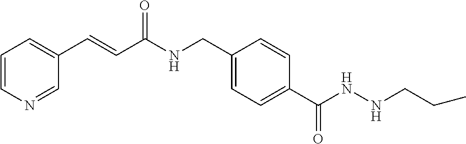 [SMILES: CCCNNC(=O)c1ccc(CNC(=O)/C=C/c2cccnc2)cc1] | 6570 |
| LEE18 | 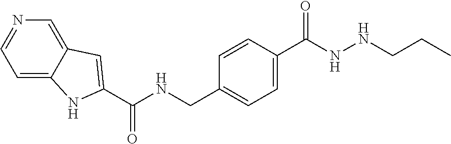 [SMILES: CCCNNC(=O)c1ccc(CNC(=O)c2cc3cnccc3[nH]2)cc1] | 1618 |
| LEE7 | 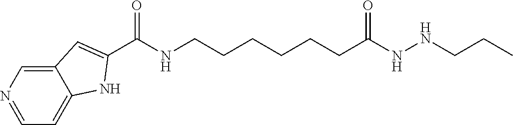 [SMILES: CCCNNC(=O)CCCCCCNC(=O)c1cc2cnccc2[nH]1] | 4555 |
| LEE19 | 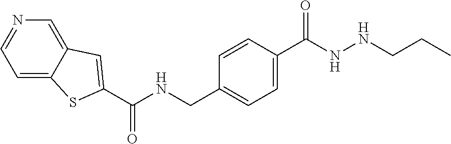 [SMILES: CCCNNC(=O)c1ccc(CNC(=O)c2cc3cnccc3s2)cc1] | >100,000 |
| LEE43 | 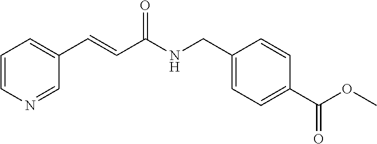 [SMILES: COC(=O)c1ccc(CNC(=O)/C=C/c2cccnc2)cc1] | 4675 |
| LEE44 | 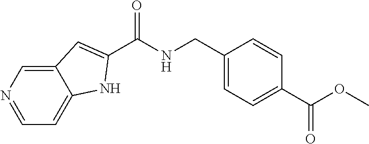 [SMILES: COC(=O)c1ccc(CNC(=O)c2cc3cnccc3[nH]2)cc1] | 6714 |
| Comparative example 1 LP411/3b | 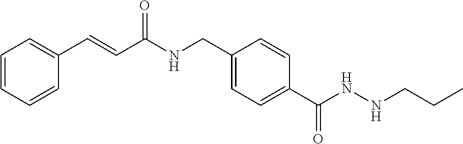 [SMILES: CCCNNC(=O)c1ccc(CNC(=O)/C=C/c2ccccc2)cc1] | >20,000 |
| Comparative example 2 LP97/13a | 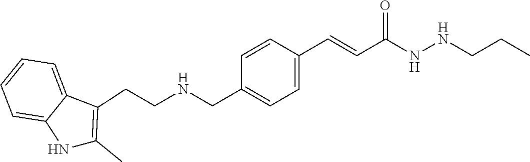 [SMILES: CCCNNC(=O)/C=C/c1ccc(CNCCc2c(C)[nH]c3ccccc23)cc1] | >20,000 |
| FK866 | | 96.11 |

ªThe data in the table are all from three independent experiments, and the values are mean values with a standard deviation of <10%.

The experimental results show that LEE12, LEE18, LEE7, LEE43 and LEE44 all exhibited micromolar level of inhibitory activity against NAMPT, and the above compounds achieved unexpected technical effects compared to compounds in comparative examples 1 and 2.

### Example 46: Median Growth Inhibitory Concentration (GI₅₀) and Median Lethal Concentration (LC₅₀) of Compounds on Tumor Cells

The median growth inhibitory concentration (GI₅₀) and median lethal concentration (LC₅₀) were determined by NCI method. Leukemia cell strains MV4-11, HL60, PL21, KASUMI-1, MONO-MAC-1 and NB-4 were cultured in an Iscove's Modified Dulbecco Medium (IMDM) containing 10% fetal bovine serum, were inoculated into a 96-well cell culture plate at a density of 10,000 cells/100 μL and cultured overnight. 6 wells were selected as Tz wells, 0.125 mg/mL Cell Titer-Blue dye was added, and the cells were cultured for 4 hours and then the fluorescence intensity at 560 nM/590 nM (excitation wavelength/emission wavelength) was read. Compounds of different concentrations were added to the remaining wells, and a 100% control group was set. After 48 hours of cultivation, 0.125 mg/mL Cell Titer-Blue dye was added and the fluorescence intensity at 560 nM/590 nM was read after 4 hours. The fluorescence intensity of the dosing group was represented by Ti, the fluorescence intensity of the 100% control group was represented by C, and the fluorescence intensity before dosing was represented by Tz.

If Ti≥Tz, the formula [(Ti−Tz)/(C−Tz)]×100 was used.

If Ti<Tz, the formula [(Ti−Tz)/Tz]×100 was used.

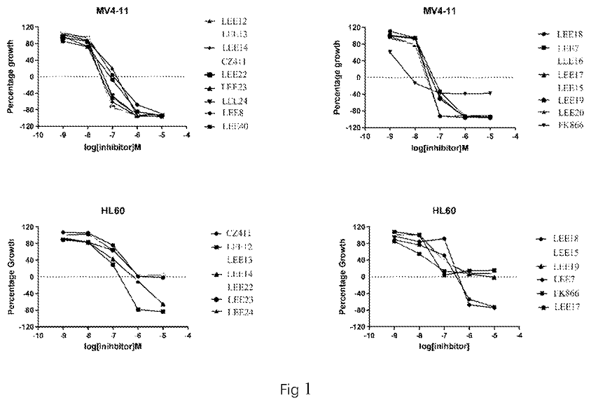

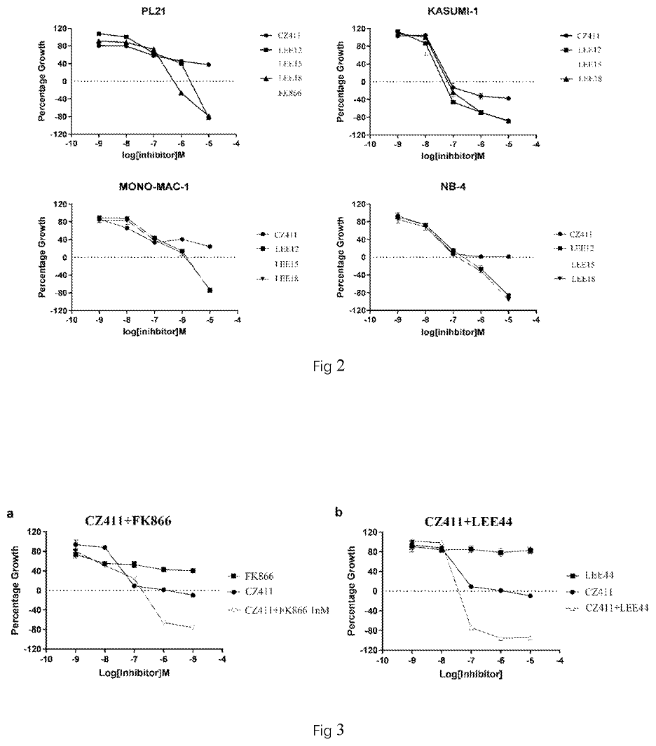

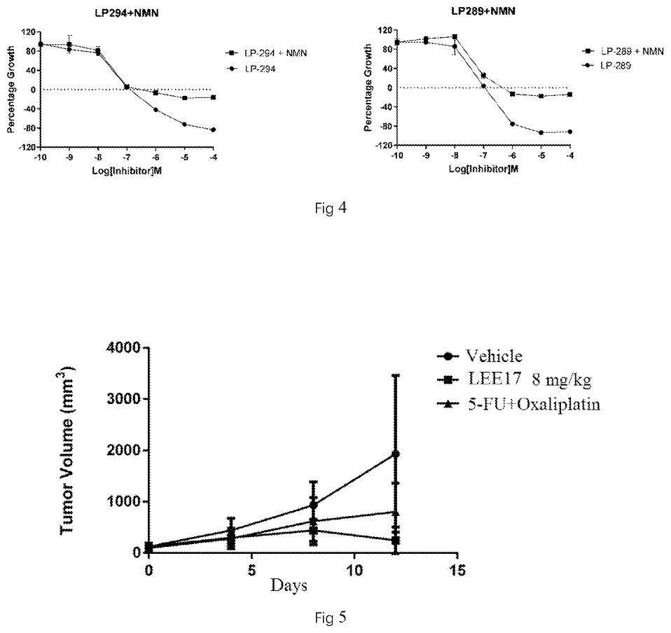

The median growth inhibitory concentration (GI₅₀) is a compound concentration of [(Ti−Tz)/(C−Tz)]×100=50, with a median lethal concentration (LC₅₀) being a concentration of [(Ti−Tz)/Tz]×100=−50. The GI₅₀ and LC₅₀ values of some compounds on leukemia cell strains MV4-11, HL60, PL21, KASUMI-1, MONO-MAC-1 and NB-4 are shown in FIG. 1, Table 3, FIG. 2, and Table 4.

TABLE 3
GI₅₀ and LC₅₀ values of some compounds on acute
myelogenous leukemia cells MV4-11 and HL60

| | MV4-11 | HL60 | MV4-11 | HL60 |
|---|---|---|---|---|
| Compound | GI₅₀ | GI₅₀ | LC₅₀ | LC₅₀ |
| No. | (nM^{a}) | (nMa) | (nMa) | (nMa) |
| Comparative | 65.99 | 120.9 | 68.80 | >10,000 |
| example 1 | | | | |
| LP411/3b | | | | |
| LEE12 | 38.31 | 42.15 | 218.5 | 590.7 |
| LEE13 | 14.98 | 128.5 | 124.7 | >10,000 |
| LEE14 | 14.29 | 63.09 | 83.61 | 6058 |
| LEE22 | 16.43 | 99.78 | 46.71 | >10,000 |
| LEE23 | 15.59 | 138.7 | 90.95 | >10,000 |
| LEE24 | 14.20 | 123.4 | 100.1 | >10,000 |
| LEE8 | 31.19 | N.Db | 455.7 | N.Db |
| LEE40 | 15.70 | N.Db | 349.2 | N.Db |
| LEE18 | 23.61 | 51.21 | 58.15 | 648.8 |
| LEE21 | 25.41 | 81.49 | 57.95 | >10,000 |
| LEE7 | 64.70 | 158.0 | 163.7 | 724.1 |
| LEE16 | 29.99 | 75.23 | 57.84 | >10,000 |
| LEE17 | 20.27 | 58.73 | 57.15 | >10,000 |
| LEE15 | 37.25 | 69.55 | 58.10 | >10,000 |
| LEE19 | 53.99 | 77.32 | 98.54 | >10,000 |
| LEE20 | 50.43 | N.Db | 109.1 | >10,000 |
| FK866 | 7.95 | 12.07 | >10,000 | >10,000 |

Note:
KF866 is an NAMPT inhibitor reported in literature.
^{a}The data in the table are all from three independent experiments, and the values are mean values with a standard deviation of <10%;
^{b}ND: not detected.

The experimental results show that in the wt-p53 cell strain MV4-11, all the tested compounds exhibited nanomolar level of GI₅₀ and LC₅₀ values, indicating that they can cause not only cell proliferation inhibition but also cell death. The activity of most compounds was significantly stronger than that of compound LP411 (3b) of comparative example 1. While in the p53-null cell strain HL60, all the tested compounds exhibited nanomolar level of GI₅₀ values, indicating that they have good anti-proliferative activity, while only LEE12, LEE14, LEE18 and LEE7 have nanomolar level of LC₅₀ values, which could lead to cell death. It is indicated that inhibiting both HDAC and NAMPT may have a synthetic lethal effect on p53-null cell strains.

TABLE 4
GI₅₀ and LC₅₀ values of some compounds on leukemia cell lines

| | | | | | MONO- | MONO- | | |
|---|---|---|---|---|---|---|---|---|
| | PL21 | PL21 | KASUM | KASUM | MAC-1 | MAC-1 | NB4 | NB4 |
| Compound | GI₅₀ | LC₅₀ | I-1 GI₅₀ | I-1 LC₅₀ | GI₅₀ | LC₅₀ | GI₅₀ | LC₅₀ |
| No. | (nM^{a}) | (nM^{a}) | (nM^{a}) | (nM^{a}) | (nM^{a}) | (nM^{a}) | (nM^{a}) | (nM^{a}) |
| Comparative | 522.8 | >10,000 | 17.80 | >10,000 | 16.26 | >10,000 | 19.50 | >10,000 |
| example 1 | | | | | | | | |
| LP411/3b | | | | | | | | |
| LEE12 | 259.2 | 3816 | 13.46 | 285.3 | 73.73 | 9032 | 22.82 | 2296 |
| LEE15 | 65.52 | >10,000 | 17.49 | 8406 | 17.95 | >10,000 | 31.87 | >10,000 |
| LEE16 | 34.28 | >10,000 | 15.29 | 5362 | 25.32 | >10,000 | 42.32 | >10,000 |
| LEE17 | 74.28 | >10,000 | 25.32 | 7325 | 33.21 | >10,000 | 51.17 | >10,000 |
| LEE18 | 109.7 | 2722 | 21.38 | 503.2 | 65.73 | 8682 | 16.47 | 1564 |

^{a}The data in the table are all from three independent experiments, and the values are mean values with a standard deviation of <10%.

FIG. 2 and Table 4 also indicate that the dual inhibitors of HDAC and NAMPT, i.e. LEE12 and LEE18, have lethal effects on p53-mutant and p53-null cell strains. The vast majority of compounds achieved unexpected technical effects compared to compounds in comparative example 1.

### Example 47: In Vivo Anti-Colon Cancer Activity of Target Compound LEE17

5-FU: 5-fluorouracil, a traditional anti-tumor chemotherapy drug; Oxaliplatin, a third-generation platinum anticancer drug, is an anti-tumor chemotherapy drug.

Colon cancer cell HCT116 was subcutaneously inoculated into the right shoulder of nude mice at 100 μL per mouse (cell count: 1.8×10⁸ cells/mL). After one week, the mice were grouped and dosed, the tumor bearing mice were grouped and administered by gavage, and the grouping is as follows:

Test group: Compound LL341, administered orally at a dose of 8 mg/kg/d, with an administration volume of 0.2 mL per animal per dose

$$Tumor inhibitory rate ( \% ) = \frac{\begin{matrix}average tumor weight in control group - \\ average tumor weight in treatment group\end{matrix}}{average tumor weight of control group} \times 100 \%$$

Relative tumor volume (RTV)=Vt/Vo

The evaluation index for anti-tumor activity is relative tumor proliferation rate T/C

$$T / C ( \% ) = \frac{Treatment group ( T ) RTV}{Negative control group ( C ) RTV} \times 100 \%$$

TABLE 7
In vivo study data results of compound
LEE17 on HCT116 tumor bearing model

| | Tumor | Relative tumor | Weight of nude |
|---|---|---|---|
| | inhibitory rate | proliferation | mice at the end of |
| Compounds | (TGI%) | rate (T/C %) | the experiment (g) |
| LEE17 | 85.5 | 14.8 | 19.04 ± 1.31 |
| 5-FU + | 23.5 | 52.3 | 19.77 ± 0.61 |
| Oxaliplatin | | | |
| Blank control | / | / | 21.39 ± 1.18 |

Note:
5-FU: 5-fluorouracil, a traditional anti-tumor chemotherapy drug; Oxaliplatin, a third-generation platinum anticancer drug, is an anti-tumor chemotherapy drug.

The experimental results show that at a dosage of 8 mg/kg/d, compound LEE17 can significantly inhibit the growth of HCT116 tumors, with an inhibitory rate of 85.5% that was significantly higher than that of positive drug 5-FU+ Oxaliplatin. At the end of the experiment, there was no significant change in the weight of the nude mice, indicating that LEE17 had a certain level of safety under the dosage of administration.

### Example 48: In Vivo Anti-Leukemia Activity of Target Compounds LEE15 and LEE16 Panobinostat: Panobinostat is a Marketed Broad-Spectrum HDAC Inhibitor

Acute myeloid leukemia cell MV4-11 was subcutaneously inoculated into the right shoulder of nude mice at an amount of 100 μL cells per mouse (cell count: 1.8×10⁸ cells/mL). After one week, the mice were grouped and dosed, the tumor bearing mice were grouped and administered by gavage, and the grouping was as follows:

Test group: compounds LEE15 and LEE16, administered at a dose of 4 mg/kg/d, with a dose volume of 200 μL/20 g per animal per dose;

Positive control group: positive drug panobinostat, administered at a dose of 4 mg/kg/d, intraperitoneal injection.

Blank control group: the same volume of PBS was given.

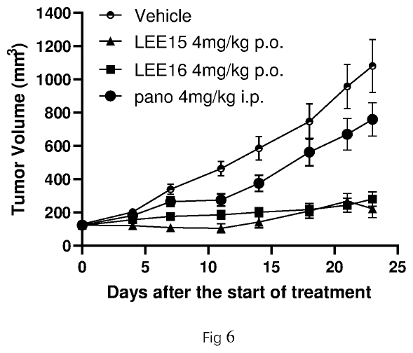

The mice were administered once a day, a tumor volume was measured every 3-4 days, an average value of each group was taken, and a tumor growth curve was drawn (see FIG. 6). On the day 23 of administration for the MV4-11 tumor bearing model, the nude mice was executed, the tumor and viscera were dissected, and at the end of the experiment, the tumor mass was weighed and a tumor inhibitory rate was calculated according to the formula. The maximum diameter (a) and minimum diameter (b) of the tumor were measured, the tumor volume (V): V=ab²/2 was calculated, and the relative tumor proliferation rate T/C (%) was calculated.

$$Tumor inhibitory rate ( \% ) = \frac{\begin{matrix}average tumor weight in control group - \\ average tumor weight in treatment group\end{matrix}}{average tumor weight of control group} \times 100 \%$$

Relative tumor volume (RTV)=Vt/Vo

The evaluation index for anti-tumor activity is the relative tumor proliferation rate T/C (%)

$$T / C ( \% ) = \frac{Treatment group ( T ) RTV}{Negative control group ( C ) RTV} \times 100 \%$$

TABLE 8
In vivo study data results of compounds LEE15
and LEE16 on MV4-11 tumor bearing model

| | | Relative tumor |
|---|---|---|
| | Tumor inhibitory | proliferation |
| Compounds | rate (TGI %) | rate (T/C %) |
| LEE15 (4 mg/kg) | 81.3 | 22.4 |
| LEE16 (4 mg/kg) | 78.4 | 25.4 |
| Panobinostat | 18.3 | 72.9 |

Note:
Panobinostat is a positive control drug, which is a marketed broad-spectrum HDAC inhibitor.

The experimental results show that at a dosage of 4 mg/kg, LEE15 and LEE16 had significant in vivo anti-acute myeloid leukemia effects, with tumor inhibitory rates of 81.3% and 78.4%, respectively. The in vivo anti-tumor activity is significantly higher than that of the positive control drug, panobinostat.

### Example 49: Pharmacokinetic Properties of Target Compounds LEE15, LEE16 and LEE17

LEE15, LEE16 and LEE17 were dissolved in 40% PEG300 and 60% H₂O. Three mice in each group were administered as a single dose by oral gavage (po) of 20 mg/kg and intravenous injection (iv) of 5 mg/kg, respectively. At the end of administration, blood samples were taken at 0.083, 0.25, 0.5, 1, 2, 4, 6, 8, and 24 hours, respectively. After the samples were prepared, parameters such as t1/2, C0, AUC, Vss, CLp, MRT, Cₘₐₓ, tₘₐₓ and F % were measured, respectively.

Po administration

| | po dose | t½ | Tₘₐₓ | Cₘₐₓ | AUCₗₐₛₜ | F |
|---|---|---|---|---|---|---|
| | (mg/kg) | (hr) | (hr) | (ng/mL) | (hr*ng/ml) | (%) |
| Comparative | 20 | 7.45 | 0.500 | 45.1 | 242 | 19.8 |
| example 2 | | | | | | |
| LP97/13a | | | | | | |
| LEE16 | 20 | 6.03 | 0.500 | 34400 | 129292 | 111 |
| LEE17 | 20 | 4.46 | 0.250 | 9463 | 27585 | 64.6 |
| LEE15 | 20 | 2.58 | 0.250 | 38800 | 128134 | 112 |
| Pano(Biomedical | 50 | 2.9 | | 116 | 126 | 4.62 |
| chromatography: | | | | | | |
| BMC 2007, 21, | | | | | | |
| 184-189) | | | | | | |

From the above metabolic data results, it can be seen that the metabolic effects of compounds LEE16, LEE17 and LEE15 are significantly better than those of compounds in comparative example 2. The aforementioned compounds LEE16, LEE17 and LEE15 have unexpected technical effects in metabolism.

The above descriptions are only preferred embodiments of the present application, but not intended to limit the present application. For those skilled in the art, various modifications and changes can be made to the presennt application. Any modifications, equivalent substitutions, improvements and the like made within the spirit and principle of the present application shall be included within the scope of protection of the present application.

1. An HDAC compound with multi-target inhibitory activity and a pharmaceutically acceptable salt, hydrate, deuterate, isomer or prodrug thereof, wherein the multi-target HDAC compound has a general formula I,
Ring E-B-L-C(O)—(NH)r-R (General formula I)
wherein, ring E is selected from 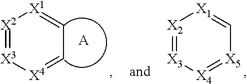
r is equal to 1 and 2, R is selected from R¹, and 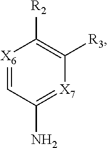
and the R¹ is selected from H, C₁₋₄ alkyl, C₃₋₅ cycloalkyl or C₁₋₂ alkyl substituted C₃₋₅ cycloalkyl;
ring A is selected from one or more substituted or unsubstituted 5-membered aromatic or heteroaromatic rings, the heteroaromatic ring contains 1 to 2 heteroatoms of N, O, or S; the substituents are H, halogen, C₁₋₂ alkyl, halomethyl, OH, OCH₃, O(CH₂)ₙCH₃, cyclopropyloxy, OC(CH₃)₃, OCH(CH₃)₂, NH₂, N(CH₃)₂, NH(CH₂)ₙCH₃, CN, N₃, etc., wherein n is from 0 to 9;
X¹ is selected from CR⁴ or N;
X² is selected from CR⁵ or N;
X³ is selected from CR⁶ or N;
X⁴ is selected from CR⁷ or N;
X⁵, X⁶ or X⁷ are independently selected from CH or N;
R² and R³ are each independently selected from H, halogen, CH₃, and OCH₃;
R⁴, R⁵, R⁶ and R⁷ are selected from H, halogen, C₁₋₂ alkyl, halomethyl, OH, OCH₃, O(CH₂)ₙCH₃, OC(CH₃)₃, OCH(CH₃)₂, cyclopropyloxy, 5 to 6-membered alkoxy, NH₂, N(CH₃)₂, NH(CH₂)ₙCH₃, CN, and N₃, wherein n is from 0 to 9;
B is selected from 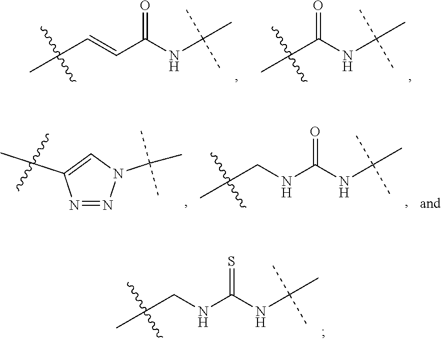 [symbol]
represents a bond connected to ring A; [symbol]
represents a bond connected to L; and
L is selected from the group consisting of C₁₋₁₄ alkyl, C₁₋₁₄ alkoxy, C₂₋₁₄ alkenyl, C₂₋₁₄ alkynyl, C₃₋₁₀ cycloalkyl, C₆₋₁₀ aryl or benzyl.

2. The HDAC compound with multi-target inhibitory activity and a pharmaceutically acceptable salt, hydrate, deuterate, isomer, or prodrug thereof of claim 1, wherein the compound of general formula I further has a structure as shown in general formula II, general formula III, general formula IV, and general formula V,

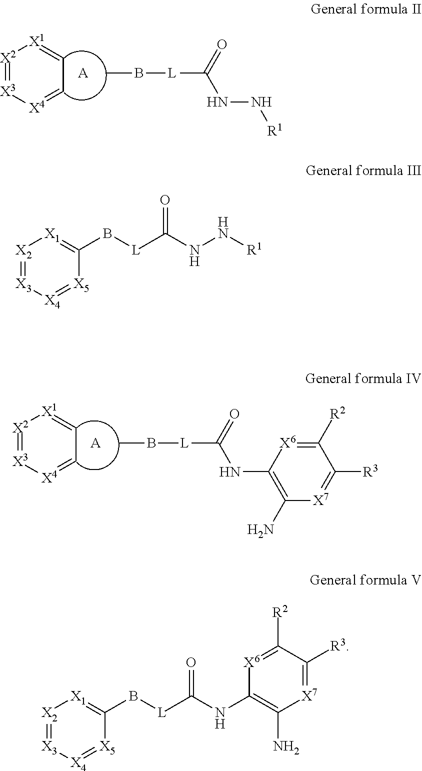

3. The HDAC compound with multi-target inhibitory activity and a pharmaceutically acceptable salt, hydrate, deuterate, isomer, or prodrug thereof of claim 1,
wherein,
ring A is selected from the following ring systems: 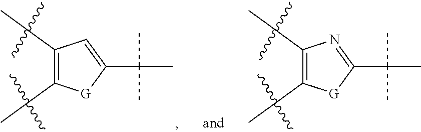
wherein,
G is selected from CH₂, NH, N(CH₂)ₙCH₃, O or S, wherein n is from 0 to 9; [symbol]
represents a bond connected to an adjacent fused ring; and [symbol]
represents a bond connected to B.

4. The HDAC compound with multi-target inhibitory activity and a pharmaceutically acceptable salt, hydrate, deuterate, isomer, or prodrug thereof of claim 1, wherein X¹, X², X³ or X⁴ is N and 1-2 of X¹, X², X³ and X⁴ are N at the same time.

5. The HDAC compound with multi-target inhibitory activity and a pharmaceutically acceptable salt, hydrate, deuterate, isomer, or prodrug thereof of claim 1, wherein X⁵ is N.

6. The HDAC compound with multi-target inhibitory activity and a pharmaceutically acceptable salt, hydrate, deuterate, isomer, or prodrug thereof of claim 1, wherein X⁶ or X⁷ is N.

7. The HDAC compound with multi-target inhibitory activity and a pharmaceutically acceptable salt, hydrate, deuterate, isomer, or prodrug thereof of claim 1, wherein R⁴, R⁵, R⁶ or R⁷ are each independently selected from H, halogen, (C₁₋₂) alkyl, halomethyl, OH, OCH₃, O(CH₂)ₙCH₃, cyclopropyloxy, OC(CH₃)₃, OCH(CH₃)₂, 5 to 6-membered alkoxy, NH₂, N(CH₃)₂, NH(CH₂)ₙCH₃, CN, and N₃, wherein n is from 0 to 9.

8. The HDAC compound with multi-target inhibitory activity and a pharmaceutically acceptable salt, hydrate, deuterate, isomer, or prodrug thereof of claim 1, wherein L is selected from linear C₁₋₁₄ alkyl or L is selected from linear C₁₋₁₄ alkoxy, wherein the C₁₋₁₄ alkyl and C₁₋₁₄ alkoxy are selected from —(CH₂CH₂O)ₘ—, —(CH₂CH₂O)ₘ—C₂H₄—, —(CH₂CH₂O)ₘ—CH₂—, —(CH₂CH₂O)ₙ—, —C₂H₄—(CH₂CH₂O)ₙ—, —CH₂—(OCH₂CH₂)n-, and —(CH₂)ₚ—O—(CH₂)_{q}—; wherein m, n, p or q is independently selected from 1, 2, 3 or 4; or when L is C₃₋₁₀ cycloalkyl, and C₆₋₁₀ aryl or benzyl, the cycloalkyl, aryl or benzyl is substituted by C₁₋₉ alkyl, C₁₋₉ alkoxy, and (C₁₋₉ alkyl)-(C═O)NH; or when L is C₃₋₁₀ cycloalkyl, the cycloalkyl contains heteroatoms such as N, O or S; or when L is aryl or benzyl, the aryl or benzyl is an aromatic heterocycle, such as N, O or S.

9. The HDAC compound with multi-target inhibitory activity and a pharmaceutically acceptable salt, hydrate, deuterate, isomer, or prodrug thereof of claim 1, wherein the compound is selected from:
(E)-N-(3-oxo-3-(2-propylhydrazino)propyl)-3-(pyridin-3-yl) acrylamide,
(E)-N-(5-oxo-5-(2-propylhydrazino)pentyl)-3-(pyridin-3-yl) acrylamide,
(E)-N-(7-oxo-7-(2-propylhydrazino)heptyl)-3-(pyridin-3-yl) acrylamide,
(E)-N-(8-oxo-8-(2-propylhydrazino)octyl)-3-(pyridin-3-yl) acrylamide,
1-(7-oxo-7-(2-propylhydrazino)heptyl)-3-(pyridin-3-ylmethyl)urea,
N-(7-oxo-7-(2-propylhydrazino)heptyl)-3H-pyrrolo[3,2-c]pyridine-2-carboxyamide 8-azido-N′-propyloctanehydrazide,
(E)-N-(2-aminophenyl)-8-(3-(pyridin-3-yl)acrylamide) octanamide
N-(2-aminophenyl)-8-azidooctanamide,
n-propyl-8-(4-(pyridin-3-yl)-1H-1,2,3-triazole-1-yl) octanehydrazide,
N-(2-aminophenyl)-8-(4-(pyridin-3-yl)-1H-1,2,3-triazole-1-yl) octanamide,
(E)-N-(4-(2-propylhydrazino-1-carbonyl)benzyl)-3-(pyridin-3-yl) acrylamide,
N-(4-(2-propylhydrazino-1-carbonyl)benzyl)-1H-pyrrolo[3,2-c]pyridine-2-carboxyamide,
N-(4-(2-propylhydrazino-1-carbonyl)benzyl)benzofuran-2-carboxyamide,
N-(4-(2-propylhydrazino-1-carbonyl)benzyl)benzo[b]thiophene-2-carboxyamide,
N-(4-(2-propylhydrazino-1-carbonyl)benzyl)-1H-indole-2-carboxyamide,
N-(4-(2-propylhydrazino-1-carbonyl)benzyl)-1H-pyrrolo[2,3-c]pyridine-2-carboxyamide,
(E)-N-(4-(2-propylhydrazino-1-carbonyl)benzyl)-3-(pyridine-2-yl) acrylamide,
(E)-N-(4-(2-propylhydrazino-1-carbonyl)benzyl)-3-(pyridine-4-yl) acrylamide,
N-(4-(2-propylhydrazino-1-carbonyl)benzyl)thiopheno[3,2-c]pyridine-2-carboxyamide,
N-(4-(2-propylhydrazino-1-carbonyl)benzyl)furan[3,2-c]pyridine-2-carboxyamide,
5-bromo-N-(4-(2-propylhydrazino-1-carbonyl)benzyl)-1H-indole-2-carboxyamide,
5-fluoro-N-(4-(2-propylhydrazino-1-carbonyl)benzyl)-1H-indole-2-carboxyamide,
6-(dimethylamino)-N-(4-(2-propylhydrazino-1-carbonyl)benzyl)-1H-indole-2-carboxyamide,
5-chloro-N-(4-(2-propylhydrazino-1-carbonyl)benzyl)-1H-indole-2-carboxyamide,
5-methoxy-N-(4-(2-propylhydrazino-1-carbonyl)benzyl)benzofuran-2-carboxyamide,
5-fluoro-N-(4-(2-propylhydrazino-1-carbonyl)benzyl)benzofuran-2-carboxyamide,
5-bromo-N-(4-(2-propylhydrazino-1-carbonyl)benzyl)benzofuran-2-carboxyamide,
5-methoxy-N-(4-(2-propylhydrazino-1-carbonyl)benzyl)-1H-indole-2-carboxyamide,
5-chloro-N-(4-(2-propylhydrazino-1-carbonyl)benzyl)benzofuran-2-carboxyamide,
5-(prop-2-yn-1-yloxy)-N-(4-(2-propylhydrazino-1-carbonyl)benzyl)benzofuran-2-carboxyamide,
5-hydroxy-N-(4-(2-propylhydrazino-1-carbonyl)benzyl)benzofuran-2-carboxyamide,
(E)-N-(4-(2-propylhydrazino-1-carbonyl)benzyl)-8-(3-(pyridin-3-yl)acrylamido) octanamide,
(E)-N-(4-(2-propylhydrazino-1-carbonyl)benzyl)-7-(3-(pyridin-3-yl)acrylamido)heptamide,
(E)-N-(4-(2-propylhydrazino-1-carbonyl)phenyl)-7-(3-(pyridin-3-yl)acrylamido) heptamide,
(E)-N-(4-(2-propylhydrazino-1-carbonyl)phenyl)-8-(3-(pyridin-3-yl)acrylamido) octanamide,
N-(4-(2-propylhydrazino-1-carbonyl)phenyl)-8-(4-(pyridin-3-yl)-1H-1,2,3-triazole-1-yl) octanamide,
methyl (E)-4-((3-(pyridine-3-yl)acrylamide) methyl) benzoate, or
methyl 4-((1H-pyrrolo [3,2-c] pyridine-2-carbonylamide) methyl) benzoate.

10. The HDAC compound with multi-target inhibitory activity and a pharmaceutically acceptable salt, hydrate, deuterate, isomer, or prodrug thereof of claim 1, wherein the pharmaceutically acceptable salt comprises inorganic acid salts selected from salts of sulfuric acid, nitric acid, hydrobromic acid, phosphoric acid, hydrochloric acid, boric acid, and sulphamic acid; or organic acid salts selected from salts of acetic acid, propionic acid, butyric acid, camphoric acid, decanoic acid, hexanoic acid, octanoic acid, carbonic acid, cinnamic acid, hydroxyacetic acid, trifluoroacetic acid, adipic acid, alginic acid, 2-hydroxypropionic acid, 2-oxopropionic acid, stearic acid, lactic acid, citric acid, oxalic acid, malonic acid, succinic acid, pyroglutamic acid, ascorbic acid, aspartic acid, phenylacetic acid, glutamic acid, benzoic acid, salicylic acid, hydroxymaleic acid, palmitic acid, cinnamic acid, isobutyric acid, lauric acid, mandelic acid, maleic acid, fumaric acid, malic acid, tartaric acid, sulfanilic acid, 2-acetyloxybenzoic acid, 2-hydroxy-1,2,3-tricarballylic acid, octanedioic acid, gluconic acid, glucuronic acid, glutamic acid, glutaric acid, formic acid, fumaric acid, mucic acid, gentisic acid, pyruvic acid, salicylic acid, methanesulfonic acid, ethylsulfonic acid, benzene methanesulfonic acid, p-toluenesulfonic acid, cyclohexyl sulfinic acid, isethionic acid, ethanedisulfonic acid, 4-(fluorenemethoxycarbonylamino)butyric acid, dichloroacetic acid, 1,2-ethanedisulfonic acid, camphore-10 sulfonic acid, 2,4-dihydroxybenzoic acid, α-ketoglutaric acid, 1-hydroxy-2-naphthoic acid, p-acetamidobenzoic acid, 2-hydroxybenzoic acid, 4-amino-2-hydroxybenzoic acid, all-trans retinoic acid, and valproic acid.

11. A pharmaceutical composition, comprising the HDAC compound with multi-target inhibitory activity and a pharmaceutically acceptable salt, hydrate, deuterate, isomer, or prodrug thereof of claim 1.

12. The pharmaceutical composition of claim 1, wherein the pharmaceutical composition is an injectable or oral preparation.

13. A process comprising preventing or treating a disease related to abnormal expression of histone deacetylase activity or NAD synthesis with the HDAC compound with multi-target inhibitory activity and a pharmaceutically acceptable salt, hydrate, deuterate, isomer, or prodrug thereof of claim 1, wherein the diseases related to abnormal expression of histone deacetylase activity or NAD synthesis are selected from malignant tumors, neurodegenerative diseases, viral infections, acquired immune deficiency syndrome, inflammation, malaria, diabetes, fungal infections, and bacterial infections, among which the malignant tumors comprise all kinds of leukemia, lymphoma, myeloma, colorectal cancer, melanoma, gastric cancer, breast cancer, ovarian cancer, pancreatic cancer, liver cancer, glioma, intracerebral tumor, kidney cancer, prostate cancer, bladder cancer, lung cancer, pancreatic cancer, ovarian cancer, skin cancer, epithelial cell cancer, nasopharyngeal cancer, epidermal cell cancer, cervical cancer, oral cancer, tongue cancer, and human fibrosarcoma.

14. A preparation method of an HDAC compound with multi-target inhibitory activity, comprising the following steps: 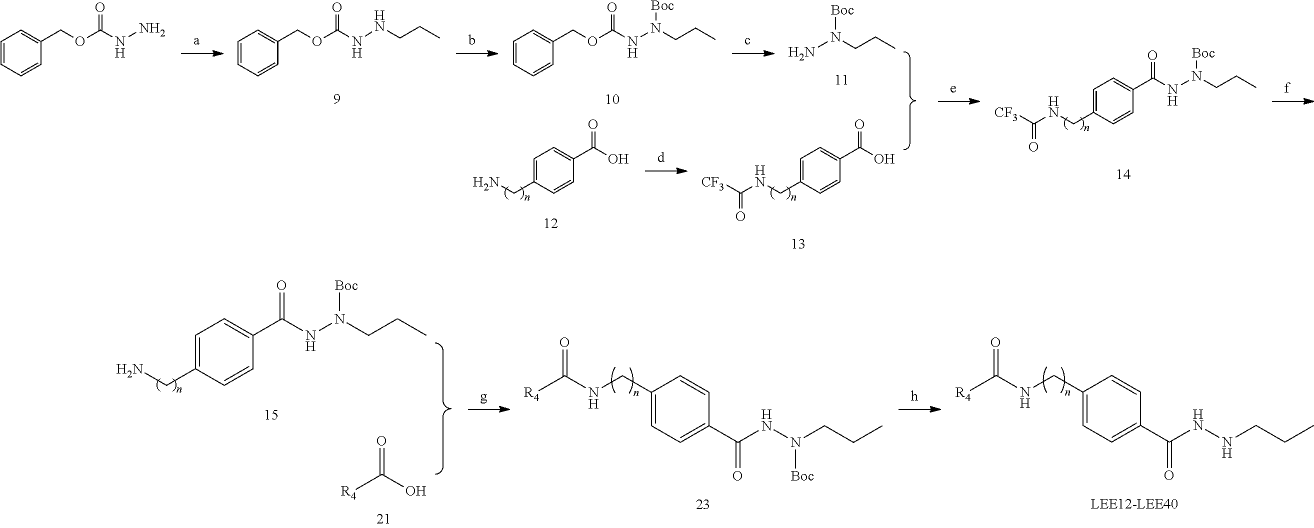 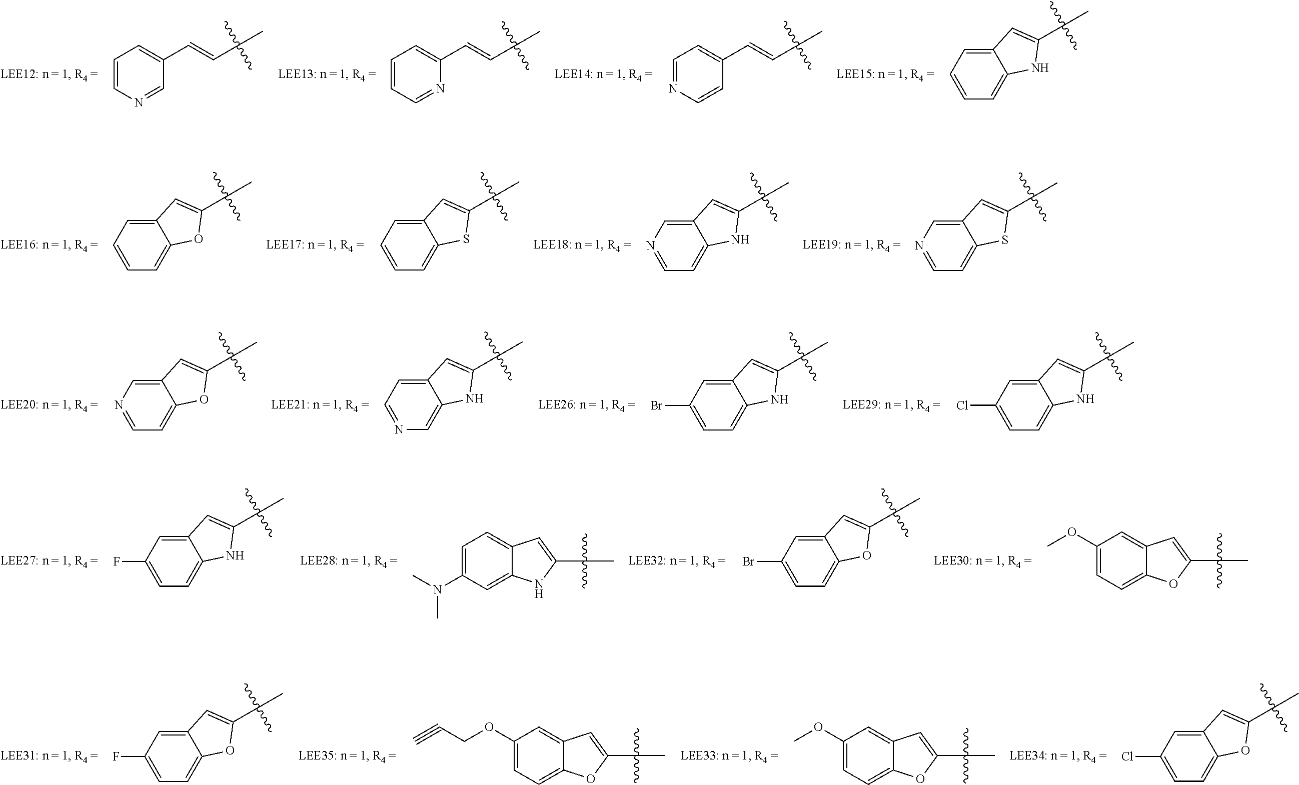 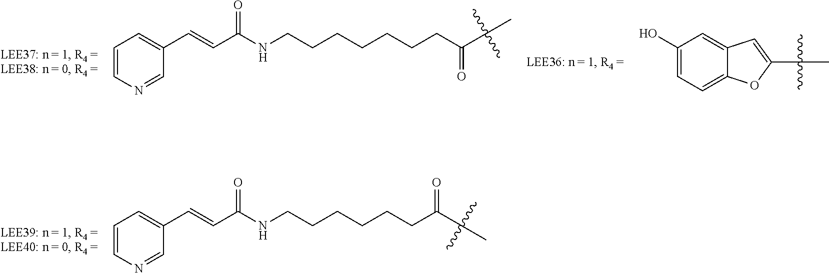 [SMILES: *C(=O)CCCCCCCNC(=O)/C=C/c1cccnc1 ; *c1cc2cc(O)ccc2o1 ; *C(=O)CCCCCCNC(=O)/C=C/c1cccnc1]
wherein the reagents and conditions are: (a) propanal, methanol, yield 98%; sodium cyanoborohydride, methanol, concentrated hydrochloric acid, methyl orange, yield 60%; (b) di-tert-butyl dicarbonate, triethylamine, ethanol, yield 85%; (c) palladium carbon, hydrogen gas, methanol, yield 85%; (d) trifluoroacetic anhydride, dichloromethane, yield 85%; (e) 1-(3-dimethylaminopropyl)-3-ethylcarbodiimide, 1-hydroxybenzotriazole, triethylamine, dichloromethane, yield 55%; (f) potassium carbonate, methanol, yield 70%; (g) O-benzotriazol-N,N,N′,N′-tetramethylurea tetrafluoroborate, triethylamine, dichloromethane, yield 55%; (h) trifluoroacetic acid, dichloromethane, triethylamine, yield 85%.

15. The pharmaceutical composition of claim 12, wherein the injectable or oral dosage forms are selected from pills, powders, lozenges, capsules, cachets, elixirs, suspensions, emulsions, solutions, syrups, aerosols (as solid or in liquid media), ointments, soft and hard gelatin capsules, suppositories, sterile injectable solutions, and sterile packaged powders
